(19) United States
(12) Patent Application Publication
(10) Pub. No.: US 2017/0355797 A1
(43) Pub. Date: Dec. 14, 2017

(54) MOLECULARLY IMPRINTED COPOLYMER COMPOUNDS AND METHODS OF PREPARATION AND USE THEREOF

(71) Applicant: University of New Hampshire, Durham, NH (US)

(72) Inventors: Casey Joseph Grenier, Pembroke, NH (US); Rudolf Seitz, Durham, NH (US)

(73) Assignee: University of New Hampshire, Durham, NH (US)

(21) Appl. No.: 15/623,094

(22) Filed: Jun. 14, 2017

Related U.S. Application Data
(60) Provisional application No. 62/350,001, filed on Jun. 14, 2016.

(51) Int. Cl. C08F 220/56; G01N 33/82; G01N 33/18; G01N 33/53; C08F 293/00; G01N 21/64

(52) U.S. Cl. CPC C08F 220/56; C08F 293/005; G01N 21/6428; G01N 33/1826; G01N 33/53; G01N 33/82; C08F 2438/03; G01N 2600/00; G01N 2021/6439

(57) ABSTRACT

The invention relates, in part, to methods to prepare and use molecularly imprinted copolymer compounds. Molecularly imprinted compounds of the invention can be used to identify the presence and/or level of a target compound that the molecularly imprinted copolymer compound of the invention was imprinted to detect. Molecularly imprinted copolymer compounds of the invention can also be used in separation methods to remove or isolate a target compound that the molecularly imprinted copolymer compound was imprinted to specifically bind.

### RELATED APPLICATIONS

This application claims benefit under 35 U.S.C. §119(e) of U.S. Provisional Application No. 62/350,001, filed Jun. 14, 2016 the content of which is incorporated by reference herein in its entirety.

### FIELD OF THE INVENTION

The invention relates, in part, to molecularly imprinted copolymer compounds, their preparation, and their use in methods such as separation and detection.

### BACKGROUND OF THE INVENTION

Sensor compounds can provide information about their environment and are also useful in separation and detection technologies. Existing sensors that are quite common are pH probes, fiber optic sensors, gas sensors, and ion-selective probes [Jordan, D. M. et al., (1987) Analytical Chem. 59 (3), 437-439; Jorgenson, R. & Yee, S., (1993) Sensors and Actuators B: Chemical 12 (3), 213-220; Lang, H. et al., (1998) Applied Physics A: Materials Science & Processing 66, S61-S64; Bühlmann, P. et al., (1998) Chemical Reviews 98 (4), 1593-1688]. Sensor technology has expanded in the past decade to include: molecular imprinting/recognition for drug delivery, surface chemistry, separation methods, and various types of paper-based sensors [Singh, B. & Chauhan, N., (2008) J. Macromolecular Science 45, 776-784; Escosura-Muniz, A. et al., (2009) Anal. Chem. 81 (24), 10268-10274; Mosbach, K., et al., (2006) J. Mol. Recognit. 19, 248-259; Abe, K. et al., (2010) Anal Bioanal Chem 398 (2), 885-893]. Certain sensors used in detection and separation technologies include biologically derived recognition agents that bind with high affinity and selectivity [Piletsky, S. & Turner, A., (2006) Molecular Imprinting of Polymers. Landes Bioscience] to a target compound. Biological sensors can be used to assess interactions such as those between DNA and proteins, receptors and ligands, and antigens and antibodies [Marazuela, M. et al., (2002) Analytical and Bioanalytical Chemistry 372 (5), 664-682]. Such sensor/target interactions have been exploited to separate mixtures of proteins and other molecules using affinity chromatography and other routine methods. Although biomolecules exhibit strong target recognition characteristics, there are difficulties associated with their use, for example, they are quite complex, unstable, difficult to synthesize, specifically bind only a unique substrate, are not tunable, and are very expensive.

### SUMMARY OF THE INVENTION

According to an aspect of the invention, molecularly imprinted polymer (MIP) compounds are provided. The compounds include a backbone monomer, two or more independently selected functional monomers, and the MIP compound also includes one or more independently selected non-covalent crosslinks and one or more covalent crosslinks. In some embodiments, the non-covalent crosslink are independently selected from: an acid-base crosslink and a hydrophobic crosslink. In certain embodiments, the percentage of the total crosslinks in the MIP compound that are covalent crosslinks is between 1% and 7%. In some embodiments, the percentage of the total crosslinks in the MIP compound that are covalent crosslinks is less than 5%. In certain embodiments, the backbone monomer is N-isopropylacrylamide (NIPAm). In some embodiments, the functional monomer is 4-vinylpyridine (4-VP) or methacrylic acid (MAA). In some embodiments, the two or more functional monomers include at least one acidic monomer and at least one basic monomer. In certain embodiments, the acidic monomer is acrylic acid (AA), methacrylic acid (MAA), or 4-Vinylphenol (4-VPH). In some embodiments, the basic monomer is 2-vinylpyridine (2VP) or 4-vinylpyridine (4VP). In some embodiments, the MIP compound is an aqueous compound. In certain embodiments, the molecular imprinting of the MIP is against a template molecule, wherein the template molecule comprises a target compound or functional fragment thereof and the MIP compound selectively binds the target compound. In some embodiments, the MIP compound further comprises a functionalized end group. In some embodiments, the functionalized group is a dithiolester. In certain embodiments, the ditholester is reduced to a thiol group. In some embodiments, the MIP compound is attached to a substrate. In some embodiments, the substrate comprises one or more of: paper, metal, plastic, nylon, cellulose, and glass. In certain embodiments, the substrate is a bead. In some embodiments, the substrate is additionally attached to a surface. In some embodiments, the MIP compound also includes a detectable label. In some embodiments, the detectable label is a fluorophore. In certain embodiments, the target compound comprises an organic molecule. In some embodiments, the target compound comprises a polar organic molecule. In some embodiments, the structure of the MIP compound is temperature dependent. In certain embodiments, at a lower critical solution temperature (LCST) of the MIP compound the structure of the MIP compound is a globular structure and at a temperature below the LCST of the MIP compound the structure of the MIP compound is a non-globular random coil structure. In some embodiments, the LCST is between: 28° C. and 38° C. In some embodiments, the LCST is between 30° C. and 34° C. In certain embodiments, the binding affinity of the globular MIP compound and the target compound is higher than the binding affinity of the non-globular random coil MIP compound and the target compound. In some embodiments, the binding affinity of the MIP compound and the target compound is modulated by the temperature of the MIP compound. In some embodiments, the binding of the MIP compound with the target compound alters one or more physical characteristics of the MIP compound. In some embodiments, the physical characteristic is one or more of: size, fluorescence, aggregation with one or more additional MIP compounds, and MIP compound phase transition. In certain embodiments, the MIP compound comprises a fluorophore and the binding of the MIP compound to the target compound alters the level of fluorescence of the fluorophore compared to the level of fluorescence when the MIP compound is not bound to the target compound. In some embodiments, the MIP compound is in a solution comprising a plurality of the MIP compounds. In some embodiments, two or more of the plurality of the MIP compounds aggregate when the solution is at a temperature above the LCST of the MIP compound. In certain embodiments, the solution additionally comprises the target compound and the binding of two or more of the plurality of the MIP compounds with the target compound inhibits the MIP compound aggregation.

According to another aspect of the invention, methods of preparing a molecularly imprinted polymer (MIP) compound of any embodiment of the aforementioned aspect of the invention are provided. The methods include: (a) preparing a pre-polymerization solution comprising: a backbone monomer, two or more independently selected functional monomers, and a solvent; (b) adding a template compound to the prepared pre-polymerization solution; (c) polymerizing the template/pre-polymerization solution to form a MIP compound; (d) separating the MIP compound from the template compound; and optionally (e) lyophilizing the separated MIP compound. In some embodiments, the backbone monomer is a N-isopropylacrylamide (NIPAm) monomer. In some embodiments, the functional monomer is 4-vinylpyridine (4-VP) or methacrylic acid (MAA). In some embodiments, the two or more functional monomers include at least one acidic monomer and at least one basic monomer. In certain embodiments, the acidic monomer is acrylic acid (AA), methacrylic acid (MAA), or 4-Vinylphenol (4-VPH). In some embodiments, the basic monomer is 2-vinylpyridine (2VP) or 4-vinylpyridine (4VP). In certain embodiments, the basic monomer is a 2-vinylpyridine (2-VP) monomer or a 4-vinylpyridine (4-VP) monomer. In some embodiments, the functional monomer is a 4-VP monomer. In some embodiments, a means of polymerizing comprises adding a polymerization solvent to the template/pre-polymerization solution. In certain embodiments, the polymerization solvent is a porogenic solvent. In some embodiments, the polymerization solvent is a non-polar solvent. In some embodiments, the polymerization solvent is 1,4-dioxane. In some embodiments, a means of polymerizing comprises a reversible addition-fragmentation chain-transfer (RAFT) method. In certain embodiments, a means for removing the template compound comprises dialysis. In some embodiments, the percentage of the total crosslinks in the MIP compound that are covalent crosslinks is between 1% and 7%. In some embodiments, the percentage of the total crosslinks in the MIP compound that are covalent crosslinks is less than 5%. In certain embodiments, the template compound is soluble in the polymerization solvent and water. In some embodiments, the template compound comprises a target compound or functional fragment thereof, and the prepared MIP compound selectively binds the target compound.

According to yet another aspect of the invention methods of identifying the presence or absence of a target compound in a sample are provided, the methods including: (a) contacting a sample with a MIP compound of any embodiment of any of the aforementioned aspects of the invention, and/or prepared using any embodiment of any of the aforementioned methods of the invention, wherein the MIP compound selectively binds a target compound; and (b) detecting the presence or absence of binding of the MIP compound and the target compound in the sample, wherein the presence of binding of the MIP compound in the sample identifies the presence of the target compound in the sample and the absence of binding of the MIP compound in the sample identifies the absence of the target compound in the sample. In some embodiments, the method also includes determining a level of the target compound identified as present in the sample.

According to yet another aspect of the invention, methods of separating a target compound from a sample are provided. The methods including: (a) contacting a sample containing a target compound with an MIP compound of any embodiment of any of the aforementioned aspects of the invention, and/or prepared using any embodiment of any of the aforementioned methods of the invention, wherein the MIP compound selectively binds the target compound forming an MIP compound/target compound complex; (b) separating the MIP compound/target compound complex from the sample, and optionally separating the target compound from the MIP compound.

### BRIEF DESCRIPTION OF THE DRAWINGS

FIG. 1A-K shows embodiments of molecular imprinting. FIG. 1A shows a molecular imprinting process demonstrating how a polymer's binding sites are formed and how that binding site is selective. FIG. 1B shows a simplified free radical polymerization mechanism involving initiation, propagation and termination. FIG. 1C shows a simplified version of the RAFT polymerization technique, involving initiation, chain growth, RAFT pre-equilibrium, re-initiation, main equilibrium and termination. FIG. 1D shows an embodiment with no crosslinks (left) present within the MIP network and an embodiment with crosslinks (right) present within the MIP network. FIG. 1E shows an example of a protein and its conformations that involve different types of non-covalent interactions. FIG. 1F shows a phase transition of poly (NIPAm) as the solution temperature is increased. FIG. 1G shows formation of a binding site. FIG. 1H shows nitrophenol used for selectivity (2-Nitrophenol and 3-Nitrophenol) and templating (4-Nitrophenol). FIG. 1I shows a fluorescein molecule and fluorescein in water under ultra-violet light. FIG. 1J shows a piezoelectric printer (above) and the prototype sensor array (below). FIG. 1K shows the attachment of MIP to a gold nanoparticle in the presence of water using sodium borohydride (above) and the attachment of MIP spin coated on to a gold coated glass slide with sodium borohydride (below).

FIG. 2A-G shows embodiments of DLS intensity. FIG. 2A provides examples of how DLS intensity works with large (above) and smaller (below) particles. FIG. 2B illustrates a vacuum distillation apparatus. FIG. 2C shows a homopolymer of n-isopropylacrylamide polymerized by free radical. FIG. 2D shows an RAFT MIP reduction to a thiol and attachment to a 20 nm gold nanoparticle. FIG. 2E shows an experimental procedure of attaching RAFT MIPs to gold nanoparticles in solution. FIG. 2F shows an equilibrium dialysis cell, 1.4 mL cell volume. FIG. 2G shows an equilibrium dialysis block before equilibration, after equilibration, and MIP binding of the template molecule.

FIG. 3A-H provides images and structures of an embodiment of a preparation method of the invention. FIG. 3A provides an absorbance spectrum near λmax=401 nm for 4-nitrophenol standards (2.5×10⁻⁵ M, 5×10⁻⁵ M, 1×10⁻⁴ M, 2×10⁻⁴ M) in 0.1 M NaOH. FIG. 3B shows a Beers law plot for 4-nitrophenol in 0.1 M NaOH. FIG. 3C shows formation of the acid-base crosslink. FIG. 3D shows hydrogen bonding of water molecules. FIG. 3E provides temperature trends for the MIPs CJG 235 and CJG 238 A. FIG. 3F shows three different versions of the benzene dimer formation. FIG. 3G shows molecular structures of the π-π stacking interact monomer units. FIG. 3H shows the theoretical observation of a dimer shift (A) and the actual absorbance of a benzyl methacrylate in a 1,4-dioxane solution (B).

FIG. 4A-O shows embodiments from preparation methods of the invention. FIG. 4A shows trace of NMR results for MIP Sensor (CJG 296 A): 1H NMR (D20, 400 MHz): δ 8.49 (br s, 2H), 7.73 (br s, 3H), 3.87 (br s 3H), 2.01 (br s), 1.92 (s), 1.56 (br s), 1.12 (br s). FIG. 4B shows trace of NMR results for NIP Blank (CJG 311 C): 1H NMR (D20, 400 MHz): δ 8.47 (br s, 2H), 7.75 (br s, 3H), 3.89 (br s 3H), 2.00 (br s), 1.56 (br s), 1.12 (br s). FIG. 4C provides a graph of diameter versus temperature for NIP blank obtained from DLS experiment. MIP concentration was 1 g/L in water and 0.1 μM fluorescein added to the second experiment. Each temperature was held there for 5 minutes and three measurements taken then averaged. Each run was taken over the course of about 12 hours. FIG. 4D provides a graph of diameter versus temperature for MIP sensor obtained from DLS experiment. MIP concentration was 0.1 g/L in water and 0.1 μM fluorescein added to the second experiment. Each temperature was held there for 5 minutes and three measurements taken then averaged. Each run was taken over the course of about 12 hours. FIG. 4E provides a schematic diagram illustrating a binding kinetics experiment. FIG. 4F shows fluorescence spectra displaying the binding kinetics of a poly (NIPAm) solution (CJG 315 A) (0.0355 mg/L) interacting with 100 nM fluorescein solution at room temperature 23° C. FIG. 4G shows fluorescence spectra displaying the binding kinetics of a NIP blank solution (CJG 311 C) (0.0355 mg/L) interacting with 100 nM fluorescein solution at room temperature 23° C. FIG. 4H shows fluorescence spectra displaying the binding kinetics of a MIP sensor solution (CJG 296 A) (0.0355 mg/L) interacting with 100 nM fluorescein solution at room temperature 23° C. FIG. 4I provides fluorescence intensity versus time of a poly (NIPAm) solution (CJG 315 A) (0.0355 mg/L) interacting with fluorescein solution (100 nM) at 32° C. FIG. 4J provides fluorescence intensity versus time of an NIP blank solution (CJG 311 C) (0.0355 mg/L) interacting with 100 nM fluorescein solution at 44° C. FIG. 4K provides fluorescence intensity versus time of a MIP sensor solution (CJG 296 A) (0.0355 mg/L) interacting with 100 nM fluorescein solution at 40° C. FIG. 4L provides fluorescence intensity versus time of a MIP sensor solution (CJG 296 A) (0.0355 mg/L) interacting with 100 nM fluorescein solution at 32° C., subtracting out the signal lost due to the dilution affect. FIG. 4M shows equilibrium dialysis results of varying the fluorescein concentration with a constant 0.035 mg/L solution of polymer sample is graphed with fluorescein intensity versus initial concentration. FIG. 4N shows the capacity of an MIP sensor, how much can be bound by 0.035 mg/L of the MIP sensor. FIG. 40 provides binding constants of the MIP sensor (CJG 296 A) with various initial concentrations of fluorescein (10-1000 nM).

FIG. 5A-M provides images relating to embodiments of methods of the invention. FIG. 5A shows the chemical structure of α-tocopherol. FIG. 5B shows the molecular structure of the MIP sensor and the formation of the binding site. FIG. 5C illustrates the hypothesized binding of the template molecule results in MIP collapse turning off fluorescence. FIG. 5D shows the proposed design details of the chemical sensor. FIG. 5E provides predicted data of the paper based printed MIP sample. FIG. 5F provides a calibration curve of α-Tocopherol. FIG. 5G provides a bar graph representing distribution ratios for the MIP sensor, NIP blank, and Poly (NIPAm) blank with both α-tocopherol and 4-hydroxycoumarin. FIG. 5H shows the chemical structure of NBD-AE, the fluorophore monomer. FIG. 5I shows the first step of the synthesis of NBD-AE. FIG. 5J shows the second and final step of the NBD-AE synthesis. FIG. 5K shows an MIP sensor present in a solvent mixture with α-tocopherol and 4-hydroxycoumarin. FIG. 5L shows a printed prototype of the designed chemical sensor under Uv/vis light at 340 nm. FIG. 5M illustrates a paper based sensor testing with various samples.

FIG. 6A-Q shows images and diagrams of results of embodiments of synthesis methods of the invention. FIG. 6A shows the attachment of MIP to an AuNP. FIG. 6B provides an SEM of gold nanoparticles ˜20 nm. FIG. 6C provides an SEM of MIP sensor (0.1 grams) stabilized gold nanoparticles (1 mL). FIG. 6D shows the manufacturing process of the MIP stabilized AuNP. FIG. 6E provides a TEM image of AuNP ˜20 nm at a 10,000 magnification setting. FIG. 6F provides a TEM image of CJG 296 A at the original concentration stated in the manufacturing procedure at a 10,000 magnification setting for the TEM image. FIG. 6G provides a TEM image of CJG 296 A at the original concentration stated in the manufacturing procedure at a 20,000 magnification setting for the TEM image. FIG. 6H shows fluorescence intensity after the stabilized MIP AuNP interact with the initial concentration of template. Error bars are the standard deviation of the values obtained over the three trials of this experiment. FIG. 6I shows the relationship of the binding constant to the initial concentration of fluorescein. FIG. 6J shows the relationship of the binding capacity of MIP stabilized AuNP at the LCST. FIG. 6K shows the fluorescence intensity after the stabilized NIP AuNP interact with the initial concentration of template. Error bars are the standard deviation of the values obtained over the three trials of this experiment. FIG. 6L shows binding kinetics experiments with AuNP interacting with 100 nM fluorescein at room temperature, ˜23° C. FIG. 6M shows binding kinetics experiments with addition of NIP blank stabilized AuNP interacting with 100 nM fluorescein at room temperature, ˜23° C. FIG. 6N shows binding kinetics experiments with MIP sensor stabilized interacting with 100 nM fluorescein at room temperature, ˜23° C. FIG. 6O shows binding kinetics experiments with AuNP interacting with 100 nM fluorescein at the LCST, 40° C. FIG. 6P shows binding kinetics experiments with addition of NIP blank stabilized AuNP interacting with 100 nM fluorescein at the LCST, 44° C. FIG. 6Q shows binding kinetics experiments with MIP sensor stabilized interacting with 100 nM fluorescein at the LCST, 40° C.

### DETAILED DESCRIPTION

The invention, in part includes preparation and use of molecularly imprinted polymer (MIP) compounds. A “molecularly imprinted polymer” (MIP) is a polymer that includes cavities (or voids) in its structure that correspond to at least a portion of one or more template compounds that are used in the preparation of the MIP. A MIP compound of the invention is prepared from a monomer mix, to which at least one template compound is added prior to polymerization of the monomers into the MIP. In some embodiments, a MIP of the invention, which is also referred to herein as an MIP compound of the invention, comprises a backbone monomer, and two or more independently selected functional monomers. As used herein the term “functional monomer” may be referred to interchangeably as a “recognition monomer”. It has been identified that the composition of monomers and types of crosslinks that are present in an MIP compound of the invention is important with respect to the stability, selectivity, affinity, and other characteristics of the MIP compound. Some embodiments of MIPs of the invention are prepared such that the MIP compound selectively binds a target compound. Methods of the invention may be used to identify and select a target compound, prepare an MIP that selectively binds that target compound.

As used herein, an MIP that is “designed against” a target compound means the target compound or a suitable template for the target compound was used to prepare the MIP and the MIP conformation results in the ability of the MIP to selectively bind with the target compound. Selectively bound MIPs and their targets may be referred to herein as an “MIP compound/target compound complex”. Such a complex may comprise an additional molecule or compound, a non-limiting example of which is a detectable label. Methods of the invention may also comprise one or more means with which to separate an MIP compound/target compound complex from a sample, and may also comprise one or more means to separate the MIP compound from the target compound.

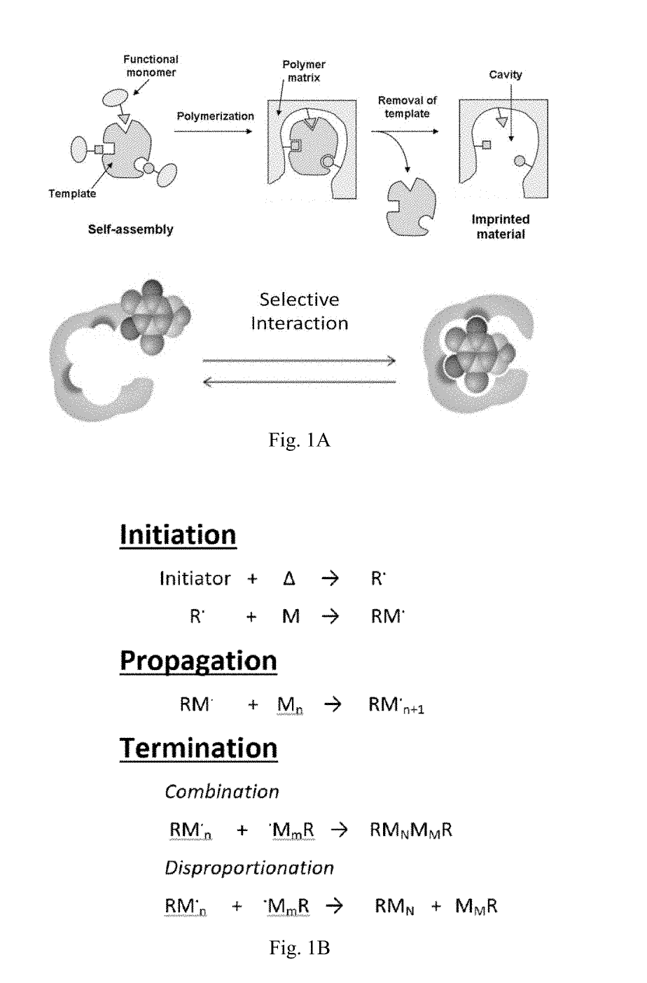

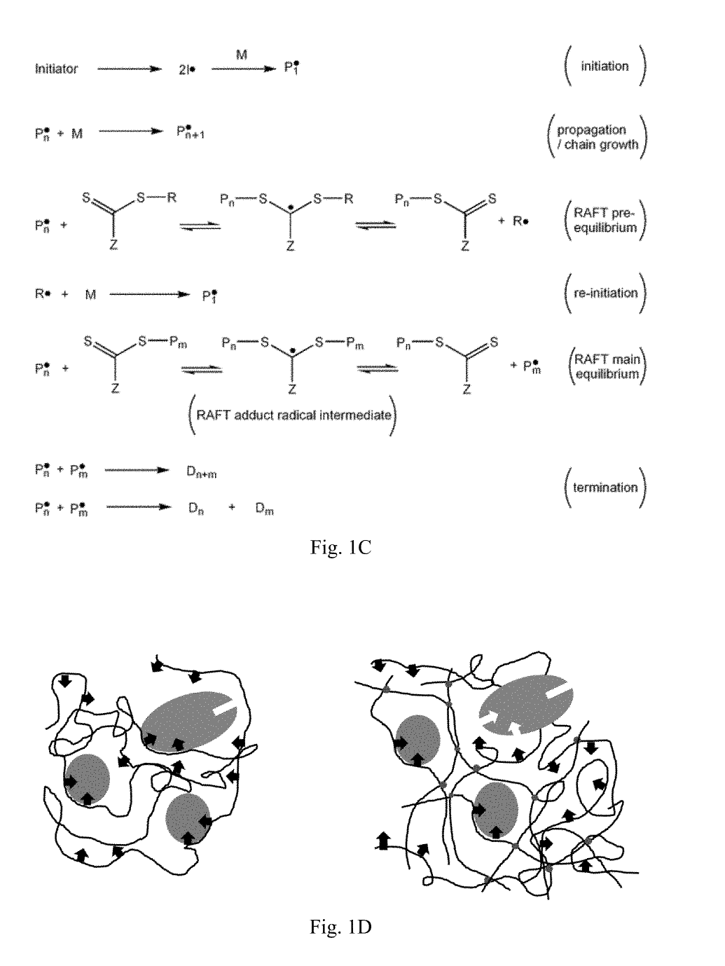

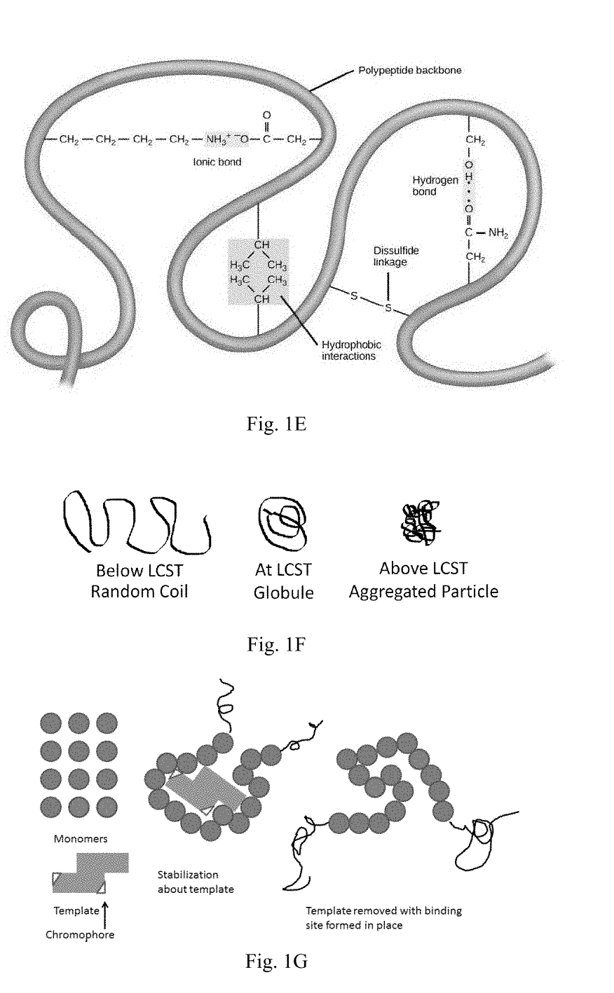

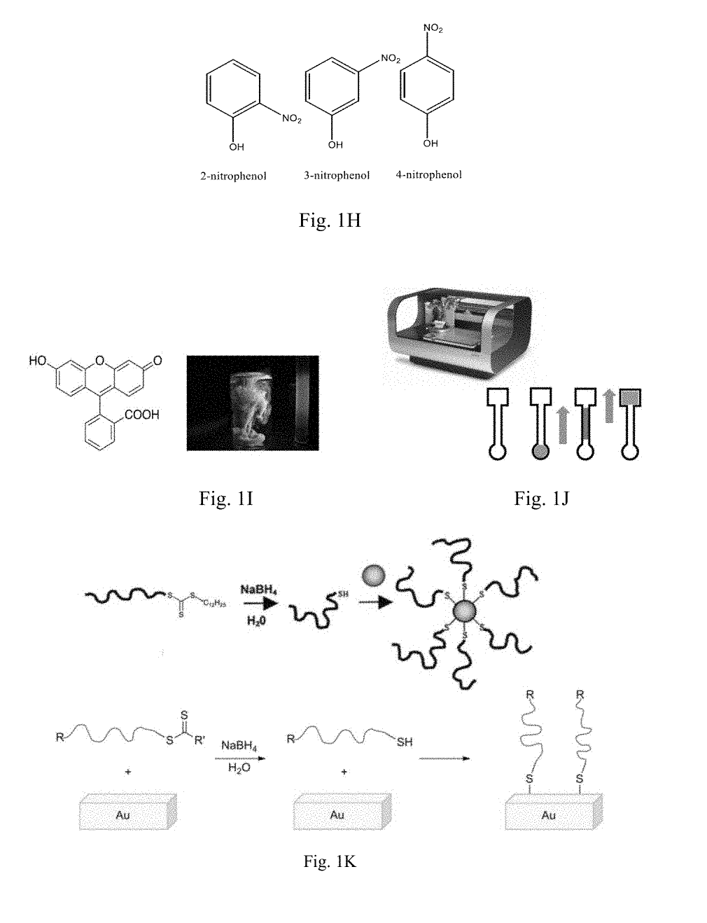

Molecular imprinting, shown schematically in FIG. 1A is based on the natural phenomenon of molecular recognition. The specific molecule to be sensed or separated is present when the polymer is synthesized. Being present allows recognition monomers to self-assemble around the template molecule through various non-covalent interactions which are illustrated in FIG. 1A. These interactions include hydrogen bonds [Sweetman, A. M. et al., (2014) Nat Com. 5], metal coordination, [Wang, F. et al., (2010) Angewandte Chemie 122 (6), 1108-1112], hydrophobic interactions [Silverstein, T. P., (1998) J. Chem. Edu. 75 (1), 116], Van der Waals interactions [Van Oss, C. et al., (1980) Colloids and Surfaces 1 (1), 45-56], π-π stacking [Sinnokrot, M. O. et al., (2002) J. Am. Chem. Soc. 124 (36), 10887-10893], and electrostatic effects [Forciniti, D. et al., (1992) Chem. Engin. Sci. 47 (1), 165-175], each of which is incorporated herein by reference in its entirety. This leads to molecular recognition of the template molecule. The resulting receptors can selectively, and with high affinity, pick out the target analyte molecule when it is present with its isomers in a complex matrix. One way to think of molecular recognition is with a “lock and key model”. The target molecule is the key, and the lock is a receptor, an arrangement of atoms that selectively recognizes the “key”. The small molecule “key” aligns with the receptor “lock” forming a coordination complex that is selective to the original interaction. A schematic illustration of formation of a binding site in a MIP compound, using templating is provided in FIG. 1G.

The invention includes, in some aspects, methods of preparing and using molecular imprinted polymers of the invention. MIPs of the invention may be used in certain aspects of the invention to recognize specific targets and for use in methods such as, but not limited to: separation of toxins from samples, removal of pollutants from water sources, chemical separations of isomers and enantiomers, removal of solids from water, preparation and use “smart” membranes that recognize specific target compounds, sorbents, and recognition elements in chemical sensors. It will be understood that as in some embodiments of the invention, a sample is a biological sample that may be obtained from cultured cells or tissues, or from a subject such as an animal. Examples of subjects include but are not limited to: a human, a non-human primate, a mammal, a vertebrate, an invertebrate, a plant, etc.

### Molecularly Imprinted Polymer Preparation

MIPs can be synthesized by several methods, including free radical polymerization and Reverse Addition-Fragment Transfer (RAFT) polymerization [Qiu, X.-P. et al., (2007) Macromolecules 40 (20), 7069-7071]. Free radical polymerization is comprised of three different steps to prepare a polymer, as shown in FIG. 1B. A radical, or active center, must first be generated. This can be done by thermal decomposition, photolysis, redox reactions, persulfates, ionizing radiation, electrochemical, plasma, sonication, or ternary initiators, or other suitable means, each of which are well known in the art. [for example: Cowie, J. M. G. & Arrighi, V. (2008) Polymers: Chemistry and Physics of Modern Materials. 3rd ed.; CRC Press: Scotland; Odian, G., (2004) Principles of Polymerization 4th ed.; Wiley-Interscience: New York; Hageman, H. J., (1985) Progress in Organic Coatings 13 (2), 123-150]. The radical addition of monomer works best on carbon-carbon double bonds from vinyl monomer units and carbon-oxygen bonds that are in aldehydes and ketones. Once the radical is formed it will attack the monomer by using one electron from the π bond to form a more stable bond with the carbon atom. The other electron will return to the second carbon atom and turn the molecule into another radical, thus beginning the polymer chain. With the chain initiated it will propagate until the termination of the radical occurs. Termination will occur by the combination of two chains with radical ends that simply combine to form one long chain. Or the chain can be terminated by one chain being abstracted to another through a hydrogen atom, which will produce a polymer with a terminal unsaturated group and a polymer with a terminal saturated group. [Antonelli, J. A. et al., (1998) Free radical polymerization].

RAFT is a form of living polymerization that produces highly monodisperse polymers and provides control over the location of co-monomers in a polymer chain by timing when monomers are introduced to the polymerization vessel. An embodiment shown in FIG. 1C, demonstrates that the radical formed by initiation (1), will add to the chain transfer agent (2i) forming the intermediate radical (2ii). This radical can then fragment to a reinitiating group (3) that can continue to polymerize by reacting with other monomer units along with the formation of a dormant chain (2iii). The rapid formation of the reversible addition-fragmentation equilibrium (4) permits the control over molecular weight and molecular weight distribution.

Certain embodiments of RAFT polymerization methods may include use of a chain transfer agent in the form of a thiocarbonylthio compound to control the molecular weight and polydispersity during a free-radical polymerization (FIG. 1C). This keeps the frequency of radical-radical terminations to a minimum. The number of growing polymer chains are less than normal free-radical because the RAFT agent traps the radical and growing polymer as a result. In aspects of the invention, Z and R groups of the RAFT may be selected for a number of reasons that affect the polymerization. The Z group primarily stabilizes the sulfur-carbon double bond and the adduct radical. This is what affects the position and rate of the elementary reaction in both the pre and main-equilibrium. Embodiments of the invention that include this technique may yield mostly linear polymers with R end groups. The R group has to stabilize the radical so that the right side of the pre-equilibrium is favored, while still being unstable enough that growth of a new polymer chain can be reinitiated. Both the temperature of polymerization and monomers present affect these parameters of a RAFT agent's kinetics and thermodynamics of the RAFT equilibria [see for example: Chiefari, J. et al., (1998) Macromolecules 31 (16), 5559-5562].

### Embodiments of Molecularly Imprinted Polymers

Molecular Imprinted Polymers (MIPs) of the invention may be categorized into three different types: polymer chains, polymer membranes, and polymer particles/beads. Imprinting can be accomplished in one-dimension, two-dimensions, or three-dimensions. These variables, in combination with the wide variety of monomers that can be polymerized to alter the polymer composition, allow the molecular imprinting technique of the invention to be used in an extensive range of conditions, solvents, templates, and polymer functions.

Certain aspects of MIP formulation of the invention involve one or more of each of the following constituents, backbone monomers, recognition monomers, and crosslinks. A backbone monomer is the monomer that will make up the majority of the polymer's molecular weight. A recognition monomer will contain the functional groups that interact with the template molecule. A recognition monomer will bind strongly and selectively to the template/target molecule. Crosslinking is essential to MIP formation using methods of the invention because it retains the size and shape of the binding site and controls some of the thermal properties of the polymer network. In certain embodiments of the invention, the template/target molecule may be a polar organic molecule that is capable of interacting with the recognition monomer during synthesis, other art-known molecules are suitable for use as template molecules and target molecules for use in methods to prepare and use MIPs of the invention. Additional non-limiting examples of template molecules and target molecules are provided elsewhere herein. Embodiments of methods of preparing and imprinting an MIP of the invention may comprise inclusion of two or more monomers, for example functional monomers and the monomers may be may be independently selected. As used herein, “independently selected” means that a monomer selected and used in the method may be the same as one or more other monomers selected or two or more different monomers may be selected. Certain embodiments of methods of preparing and imprinting an MIP of the invention may comprise inclusion of one or more independently selected non-covalent crosslinks and one or more independently selected covalent crosslinks. Thus, in some embodiments of the invention, non-covalent and covalent crosslinks are independently selected from other non-covalent and covalent crosslinks.

In methods of preparing MIPs of the invention, a template molecule will interact with the recognition monomer before polymerization and the polymer will form around the template molecule, creating a shape-dependent cavity. In some aspects of the invention, an initiator may be used to form a radical/active center that can initiate polymerization. Chain transfer agents may also be included in methods of the invention, and such agents allow for a living polymerization to take place so that uniform chains/networks can be formed. In certain embodiments of the invention, polymerization of the MIP occurs in a solvent that is appropriate for all monomer molecules and the template. An non-limiting example of a non-hydrogen bonding solvent that may be used in methods to prepare an MIP of the invention is 1,4-dioxane. 1,4-dioxane can be suitable as a solvent at least in part, because hydrogen bonding is a very common interaction between template and recognition monomer.

Additional non-limiting examples of solvents are provided include: porogenic solvents, dimethyl sulfoxide, tetrahydrofuran, acetonitrile, acetone and 1,4-dioxane. It will be understood that other solvents known are suitable for use in methods to prepare an MIP of the invention. Template-solvent interactions competing with template-recognition monomer interactions are unwanted. The term “removal solvent” is used herein in reference to a solvent system that will dissociate the template/target molecule from its binding site after the polymerization is complete, but will still leave the binding site intact. In addition to solvents, bonding solvents, and/or removal solvents described herein, additional solvents, bonding solvents, and removal solvents are suitable for use in methods to prepare embodiments of MIPs of the invention.

In some aspects of the invention, a polymerization vessel is a container in which one or more of each of: monomers, template molecules, initiators, and polymerization solvents are combined in desired ratios and heated for a determined period of time. The resulting product is polymer strands that have template molecule non-covalently bound to the recognition sites. After the template is removed, the polymer will “remember” the conformation of the template against which the polymer was formed. Each site of the MIP that was initially occupied by template becomes a binding site and can be used in methods of the invention to bind a desired target molecule for which the MIP was designed and prepared. Crosslinking as described herein allows the binding sites in the MIP to retain the specific size and conformation of the template molecule. Maintaining the size and/or conformation of the template molecule permits the MIP when contacted with a target molecule for which the MIP was designed, (also referred to herein as the target molecule that the MIP of the invention was designed against), to bind the target molecule. As used herein contacting an MIP of the invention with its target molecule may occur in a solution, a biological sample, an environmental sample, etc. In some aspects of the invention, an MIP is attached directly or indirectly to a surface and in other aspects of the invention an MIP of the invention is free in a solution, sample, etc. Methods of the invention, in some embodiments, include methods in which an MIP of the invention has sufficient opportunity to contact the target against which the MIP was designed, such that if the target is present, binding between the MIP and target may occur. It has been identified that use of types of different crosslinks and/or percentages of different crosslinks determines characteristics of an MIP of the invention. Types and percentages of crosslinks can be varied to produce MIPs of the invention that have specific functionality such that the MIP can bind to the target molecule for which the MIP was designed.

FIG. 1D provides a non-limiting example of two MIPs with the same chemical formulation, except that one has no crosslinking monomer within the polymer network. Both MIPs were imprinted with the same template. After template has been removed, the MIPs were placed in an environment that has both template and its stereoisomer. It can be seen that the un-crosslinked MIP binds both the template and its isomer; however, the crosslinked MIP just binds the templated molecule. This example illustrates how the crosslink holds the binding site in place, which enables an MIP of the invention to selectivity interact with its template.

Use of covalent crosslinks within an MIP or MIP network of the invention results in selective binding of the target molecule, but causes this to occur very slowly. Conversely, MIPs with little or no crosslinking binds template more rapidly, but with little selectivity. The presence of crosslinks causes the polymer to be ridged, which hinders binding of the MIP. In certain embodiments of preparation methods of the invention, binding kinetics range may be up to 1, 2, 3, 4, 5, 6, 7, 8, 9, 10, 11, or more hours, and in certain aspects of the invention binding kinetics range from 10 to 20 hours, 12 to 24 hours, 10 to 30 hours, 15-40 hours in length. These long times indicate that rigid polymer networks cannot easily move to expose the binding site. Unlike MIPs of the invention, most, if not all, of these crosslinks in previous MIPs have been covalent bonds.

Proteins and other biological macromolecules do not use covalent bonds to hold their various forms or to bind to different receptors. Proteins primarily have this type of non-covalent bonds for their different conformations and folds. Non-covalent “bonds” are different because they do not involve shared electrons. Types of non-covalent interactions include: electrostatic (ionic and hydrogen bonds), it-stacking effects, van der Waals forces, and hydrophobic effects. An example of non-covalent interactions within a protein can be seen in FIG. 1E. These interactions are critical in holding these folds in place for biological receptors to maintain their conformation because proteins are less rigid and they are capable of much faster binding kinetics than previously known MIPS. It has now been identified that high amounts of crosslinking will create an insoluble solid phase particle. Most polymers that incorporate high levels of crosslinking are not soluble in aqueous solutions and usually require an organic solvent. This is unlike biological receptors such as proteins and antibodies, which are soluble in water. Certain embodiments of the invention include methods of molecular imprinting to prepare MIP compounds that are stable polymers with selective binding properties and that may be soluble in aqueous solutions.

### MIPs Based on Inclusion of Poly (NIPAm)

The invention, in some aspects provides MIPs based on poly (NIPAm). Such MIPs of the invention offer an alternative approach to conventional MIP preparations. Poly (NIPAm) in the presence of water will undergo a phase transition from a swollen hydrated polymer under a solution temperature of 32° C., to a dehydrated shrunken state above 32° C. solution temperature. At temperatures below the lower critical solution temperature (LCST), hydrogen bonding involving the amide group is strong enough to keep poly (NIPAm) in solution. This state is called a random coil and is illustrated in FIG. 1F. Although not wishing to be bound by a particular theory, at the higher temperatures, above the 32° C. LCST, the hydrogen bonding with the water molecules surrounding the polymer chain is not strong enough to overcome the hydrophobic interactions between the aliphatic polymer backbone, isopropyl groups, and the water. Increasing the temperature above the LCST will cause the polymer to form globules with hydrophobic interactions dominating the center and hydrophilic interactions on the surface of the polymer globule. If the temperature is increased further above the LCST, this will cause the polymer chains to go from a globule state to a clump of chains forming an aggregated solid polymer particle that is completely phased out of the aqueous solution. Poly (NIPAm) will lose about 90% of its water volume when the temperature is raised above 32° C. [see for example: Mukae, K. et al., (1994) Colloid Polym Sci 272 (6), 655-663].

In certain embodiments of methods of the invention, a polymer returns to its polymerized conformation, as shown in FIG. 1F. Poly (NIPAm) has a known LCST at 32° C. which can be altered depending on the co-monomer added and the amount present within the polymer. These polymers can be tunable to physiological temperature, 37° C., making them well suited for use in MIPs of the invention, for example, for use in methods to detect, sense, or separate target compounds from samples, a non-limiting example of which is a biological fluid such as a bodily fluid or tissue suspension obtained from a subject such as a human or other mammal, vertebrate, invertebrate, etc. A biological sample may also be a sample from a body of water, a soil sample, a sample of suspected organic contaminant, etc. In some aspects of the invention a sample is not a biological sample, but rather is a chemical sample, a manufacturing sample, etc. It will be understood that a sample may originally be obtained in liquid form or solid form or mixed form and can be suspended into a liquid state, (for example though not intended to be limiting: an aqueous state), for contacting with an MIP of the invention.

### Monomer and Crosslink Percentages

In certain aspects of the invention, an MIP compound of the invention is prepared such that the resulting MIP compound comprises one or more acid-base crosslinks and one or more covalent crosslinks. An MIP compound of the invention may comprise different types of crosslinks between its component monomers, and a percentage of the total crosslinks are acid-base crosslinks and a percentage of the crosslinks are covalent crosslinks. For example, covalent crosslinks may make up between 0.5% and 10%, 0.5% and 9%, 0.5% and 8%, 0.5% and 7%, 0.5%, and 6%, 0.5% and 5%, 0.5% and 4%, 0.5% and 3%, 0.5% and 2%, or 0.5% and 1% of the total crosslinks in the MIP compound. In certain aspects of the invention, the percentage covalent crosslinks is less than 10%, less than 9%, less than 8%, less than 7%, less than 6%, less than 5%, less than 4%, less than 3%, less than 2%, or less than 1% of the total crosslinks in the MIP compound.

The percent of different types of crosslinks results from the amount of different monomer types included in an MIP compound of the invention. For example, the overall percentage of acid monomers and base monomers may determine, at least in part, the percentage of acid-base crosslinks in a prepared MIP compound of the invention. Examples of various monomer mixtures with different percentages of different monomers are provided herein, including in the Examples section. In some aspects of the invention, the percentage of a backbone monomer in the total monomers of an MIP compound of the invention is greater than 70%, 75%, 80%, 85%, 90%, 91%, 92%, 93%, 94%, 95%, 96%, 97%, or 98%. A non-limiting example of a backbone monitor that can be used in some embodiments of MIP compounds of the invention is an N-isopropylacrylamide (NIPAm) monomer.

Other monomers that may be included in certain embodiments of MIP compounds of the invention include, but are not limited to: acid monomers, basic monomers, recognition monomers. Non-limiting examples of acid monomers, which are also referred to herein as “acidic monomers” are: acrylic acid (AA) and methacrylic acid (MAA). Non-limiting examples of base monomers, also referred to herein as “basic monomers” are 2-vinylpyridine (2-VP, 2VP) and 4-vinylpyridine (4-VP, 4VP). In certain aspects of the invention, 4-VP is included in an MIP compound in which it functions as recognition monomer. In certain aspects of the invention, MBAm is also included, which results in covalent crosslinks in the resulting MIP compound of the invention. Additional acid monomers and base monomers suitable for use in MIPs and methods of the invention are known in the art.

### Imprinting

An aspect of preparing an MIP compound of an embodiment of the invention comprises imprinting the MIP against a template compound. As used herein, a template compound may be comprised of one or more molecules. It will be understood that a template compound that comprises one molecule may be referred to herein as a template molecule. A template compound may be selected because it includes all or a functional portion of a target compound that is of interest for selective binding by the prepared MIP compound of the invention. As used herein a target compound may be comprised of one or more molecules. It will be understood that a target that comprises one molecule may be referred to herein as a target molecule.

The presence, absence and/or level of binding between an MIP of the invention and its target molecule may be determined using methods described herein and other suitable art-known methods. In some aspects of the invention, binding of an MIP compound of the invention to its target compound may alter one or more characteristics of the MIP compound. Non-limiting examples of characteristics of the MIP compound that may be changed by binding to its target are: physical characteristics of the MIP compound, such as, but not limited to: the MIP's size, fluorescence, the MIP's aggregation with one or more additional MIP compounds, and MIP compound phase transition. In some aspects of the invention, an MIP compound comprises a fluorophore and binding of the MIP compound and its target compound results in a change in fluorescence emission by the fluorophore. Changes in a fluorescence emission can be detected using routine detection methods and the presence, absence and/or changes in fluorescence levels with and without binding between the MIP of the invention and its target compound. Determinations of amounts of bound and unbound MIP can be used to assess presence, absence, level of its target compound in a sample that is tested using an MIP of the invention. It will be understood that the determination of the presence, absence, and/or amount/level of a target compound using an MIP compound of the invention may include a fluorescent means or another suitable means with which to make the assessment.

A template compound need not be the target compound of interest itself but may comprise a structure that is sufficient to imprint the MIP compound thereby creating an imprinted region of the MIP compound that recognizes the target compound of interest. The target compound of interest includes at least one region that matches the imprinting compound and therefor is recognized and “fits” the imprinted region of the MIP compound of the invention. In some aspects of the invention, a template compound may be a portion of, or may be an entire target compound. As used herein a portion of a target compound that can be used as a template compound may be referred to as a “functional fragment” of the target compound. In some aspects of the invention, a functional fragment of a target compound may be a compound or molecule that has at least in part an identical 3-D structure and/or profile of a portion of the target compound.

In certain aspects of the invention, a template compound may share one or more structural characteristics with a target compound of interest that permits it to function as a template for that target compound, and in some embodiments of the invention, the template compound is the target compound itself or a portion thereof. In certain aspects of the invention, the template compound is not the target compound itself or a portion thereof. In some aspects of the invention, a template compound may be, or may comprise, the complete target compound or it may be, or may comprise: a functional fragment of the target compound; a derivative of the target compound, a mimic of the target compound; a peptidomimetic of the target compound, and the like. In certain aspects of the invention, an intended target compound may comprise a specific series of two, three, four, or more amino acids and a template compound for that target may include the specific series of the amino acids and may also comprise some or all of the remaining portions of the target compound, and/or may comprise other components that are not part of the target compound. Additional template compounds suitable for preparing MIP compounds of the invention that recognize and bind to a target compound of interest will be known in the art.

Non-limiting examples of target molecules (which may also be referred to herein as target compounds) against which an MIP of the invention can be designed and prepared include one or more of: a protein, a polypeptide, a protein complex, a synthetic organic compound, a naturally occurring organic compound, a synthetic inorganic compound, a naturally occurring inorganic compound, an enzyme, a receptor molecule, a vitamin, a toxin, etc. Non-limiting examples of target molecules and template molecules that correspond to all or part of a target molecule of interest and can be used in methods to prepare an MIP of the invention are provided herein. It will be understood that additional art-known target molecules/compounds and template molecules/compounds are suitable for use in methods of the invention.

### Substrates and Surfaces

In some aspects of the invention, an MIP compound is attached to a substrate. Examples of attachment means include, but are not limited to covalent attachment, attachment via a reduced end group on an MIP of the invention. For example, though not intended to be limiting, an MIP of the invention may include an end group (such as a dithioester end group) that can be reduced (for example to a thiol end group) resulting in attachment of the MIP to a particle or surface via the reduced group. An example of an agent that may be used to reduce a dithioester end group, resulting in attachment to a surface is sodium borohydride. In some aspects of the invention, the thiol end group attaches the MIP to a metal particle or metal surface, and in some aspects of the invention the metal is gold. It will be understood that other art-known attachment means can be used to attach an MIP of the invention to a surface. As used herein in regard to attaching MIPs of the invention, the term “surface” and “substrate” may be used interchangeably. Non-limiting examples of a surface or substrate to which an MIP may be attached is a surface or substrate comprising one or more of: paper, metal, plastic, nylon, cellulose, and glass. The form of a surface or substrate can vary depending on the method of the invention in which it is used. For example, though not intended to be limiting, a surface or substrate may be in the form of a bead, slide, paper, or other form and in certain aspects of the invention, a surface or substrate may be further attached to a second surface.

### Labels and Additional Compounds

In some aspects of the invention, an additional compound or molecule may also be attached to an MIP compound of the invention. Non-limiting examples of additional compounds are: detectable labels. Detectable labels may include fluorescent molecules, enzymatic labels, radiolabels, or other art-known labels that are suitable to determine the presence or absence and/or level of an MIP of the invention. Determination of presence, absence, and/or level of an MIP of the invention can comprise methods described herein and also art-known methods to detect and quantitate detectable labels.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

### Materials and Equipment for Examples 1-6

(A) Reagents are listed by their sources. Abbreviations are in the parentheses in bold. The following list of materials and chemicals were used in the following experiments:
   Sigma Aldrich (St. Louis, Mo.)
Methacrylic acid 99% purity (MAA). Vacuum distilled then passed through a column of basic alumina and inhibitor remover column to remove inhibitor.
4-Vinylpryidine 95% purity (4VP). Vacuum distilled then passed through a column of basic alumina and inhibitor remover column to remove inhibitor.
N,N′-Methylenebisacrylamide 99% purity (MBAm). Recrystallized from methanol (3 times) to remove inhibitor.
Benzyl Methacrylate 96% purity (BM). Vacuum distilled then passed through a column of basic alumina and inhibitor remover column to remove inhibitor.
1-Pyrenemethyl Methacrylate 99% purity (PMA). Recrystallized from methanol (3 times) to remove inhibitor.
2-Acrylamido-2-Methyl-1-Propanesulfonic Acid Sodium Salt Solution 50% (w/w) in H2O (AMPS). Passed through a column of basic alumina to remove inhibitor.
[3-(Methacryloylamino) propyl] Trimethylammonium Chloride Solution 50% (w/w) in H2O (MPTA). Passed through a column of basic alumina to remove inhibitor.
The following reagents were used as received from manufacturer/distributer, Sigma Aldrich (St. Louis, Mo.)
2-(Dodecylthiocarbonothioylthio)2-methylpropanoic acid 97% purity (DDMAT); 2,2′ Azobisisobutyronitrile 98% (AIBN); Inhibitor remover beads; 2-Nitrophenol 99% purity (2N); 3-Nitrophenol 99% purity (3N); 4-Nitrophenol 99% purity (4N); Fluorescein, free acid (FL); Alpha-Tocopherol 96% purity (Vitamin E); 4-Hydroxycoumarin 98% purity (4HC); Ascorbic Acid 99.9% purity (Vitamin C); 4-Chloro-7-Nitrobenzofurazan 98% purity (NBD-Cl); Ethanolamine, ACS reagent, ≧99% (ETA); Sodium Hydroxide, ACS reagent, ≧97% pellets (NaOH); 1,4-Dioxane, 99.8% anhydrous; Dimethyl Sulfoxide, ACS Grade (DMSO); Methanol, ACS Grade; Ethanol, ACS Grade; Acetone, ACS Grade; Chloroform, ACS Grade; Hexane, ACS Grade; Dichloromethane, ACS Grade; Acetonitrile, ACS Grade; Ethyl Acetate, ACS Grade.
4-(2-Acryloyloxyethylamino)-7-Nitro-2,1,3-Benzoxadiazole (NBD-AE). Synthesized.

### Spectrum Labs (Florence, S.C.)

Flat Dialysis Sheet Spectra/Por 3, MWCO 3.5 kD; Dialysis Tubing Spectra/Por 2, MCWO 12-14 kD; Dialysis Clips 78 millimeters, weighted and unweighted.

### EMD Millipore (Billerica, Mass.)

Acetic Acid; Hydrochloric Acid

### Tokyo Chemical Industry (Tokyo, Japan)

N-isopropylacrylamide 99% purity (NIPAm). Recrystallized from hexane (3 times) to remove inhibitor.
2-Isopropenylnaphthalene 98% purity (2IPN). Recrystallized from chloroform (3 times) to remove inhibitor.

### Alfa Aesar (Ward Hill, Mass.)

Benzyl Acrylate 97% purity (BA). Vacuum distilled then passed through a basic alumina and inhibitor remover column to remove inhibitor.
Basic Alumina Activated. Used as received.

### Wako Chemicals (Richmond, Va.)

Methanol, ACS Grade; Ethanol, ACS Grade; Acetone, ACS Grade; Chloroform, ACS Grade; Hexane, ACS Grade; Dichloromethane, ACS Grade; Acetonitrile, ACS Grade; Ethyl Acetate, ACS Grade; Acetic Acid; Hydrochloric Acid; Nitric Acid

TABLE 1A
Apparatus and Characterization

| | |
|---|---|
| Nuclear Magnetic Resonance Spectroscopy | NMR |
| Gel Permeation Chromatography | GPC |
| Transition Electron Microscopy | TEM |
| Scanning Electron Microscopy with focused Ion Beam | FIB-SEM |
| Ultra-violet/Visible Spectroscopy | Uv/Vis |
| Fluorescence Spectroscopy | FS |

TABLE 1B
Abbreviations

| Abbreviation | Instrument Brand and Module |
|---|---|
| DLS | Malvern Zetasizer Nano NS |
| NMR | Varian 400 MHz Mercury Liquid State NMR |
| GPC | Agilent 1260 GPC with a Polymer Lab Aqueous SEC |
| | Column |
| TEM | Zeiss/LEO 922 Omega TEM |
| FIB-SEM | Tescan Lyra3 GMU FIB SEM |
| Uv/Vis | Cary 60 Uv/Vis Spectrophotometer |
| FS | Cary Eclipse Fluorescence Spectrophotometer with a Peltier |
| | thermostatted single cell holder |

TABLE 1C
Abbreviations and use and function listing

| Abbreviation | Instrument Use and Function |
|---|---|
| DLS | Measure the particle size for all polymers. When done over a |
| | temperature trend its primary use was to determine an LCST value |
| NMR | Was used to determine the polymer's structure by identifying the |
| | different hydrogen atoms present. Exploits the magnetic properties of |
| | certain atomic nuclei. It determines the physical and chemical |
| | properties of atoms or the molecules in which they are contained. |
| GPC | A size exclusion chromatography technique that allows us to separate |
| | the polymer based on the size of the polymer. Used to determine |
| | molecular weights and their polydispersity index. |
| TEM | Visually used to see how the MIP was interacting with the AuNP and |
| | to see the attachment of the MIP to an AuNP. A microscopy technique |
| | in which a beam of electrons is transmitted through an ultra-thin |
| | specimen, interacting with the specimen as it passes through it. |
| FIB-SEM | Primarily useful for giving a three-dimensional image of the surface of |
| | the species/samples. Samples are imaged with a beam of electrons by |
| | scanning the beam across the sample. This creates an image of the |
| | surface of the sample with exceptional depth of the field. This is |
| | created by the detection of secondary electrons that are released from |
| | the samples as a result of it being scanned by very high energy |
| | primary electrons. |
| Uv/Vis | Refers to absorption spectroscopy or reflectance spectroscopy in the |
| | ultraviolet-visible spectral region. This means it uses light in the |
| | visible and adjacent (near-UV and near-infrared [NIR]) ranges. |
| FS | Both an excitation spectrum (the light that is absorbed by the sample) |
| | and/or an emission spectrum (the light emitted by the sample) can be |
| | measured. The concentration of the analyte is directly proportional |
| | with the intensity of the emission. |

A Branson model 1210 sonicator was used for reagent dissolution, nanoparticle mixing and sonication. A Buchi RE111 Rotovapor was used to evaporate the solvents. Separation of gold nanoparticles coated with polymer/MIP from the solution was performed with an Eppendorf Centrifuge 5415 D (13,200 rpm) Microcentrifuge 26 Tube Holder. A Labconco FreeZone 1 Liter Bench Top Freeze Dry System coupled with a stainless steel tower with four ports and variable glass containers were used to remove the water from the polymer solution yielding a polymer powder. A Synthware Four Port Greased Schlenk line coupled with Alcatel oil pump was used to perform freeze-pump-thaw degassing and vacuum distillation using a 14 centimeter length and 2 centimeter width distillation column coupled with a 20 centimeter water condenser. Dialysis tubing, Sigma Aldrich MWCO of 12,000 to 14,000, was used to purify MIPs. Sigma Dialysis Sheets with MWCO of 3,500 was used in the equilibrium dialysis cell by Bel-Art, 1.4 mL volume. UNH Machine Shop made HDPE equilibrium dialysis cells mocked up from the Bel-Art model in the same size and volume amounts.

### Dynamic Light Scattering

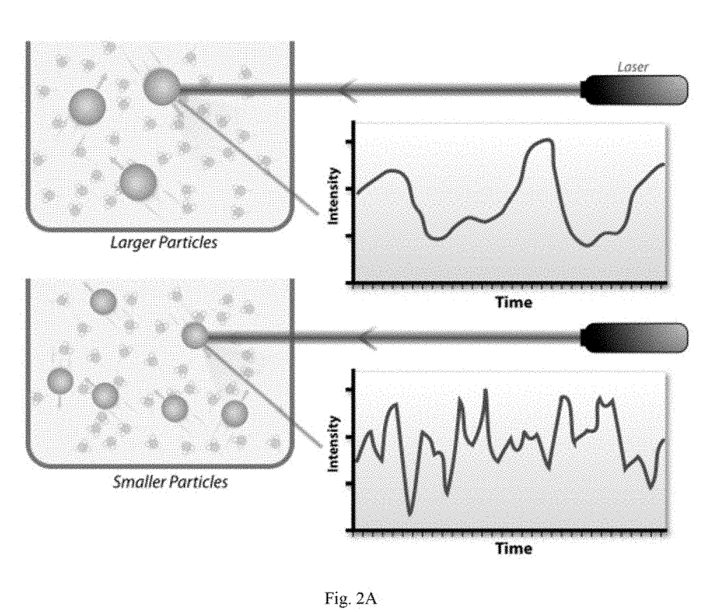

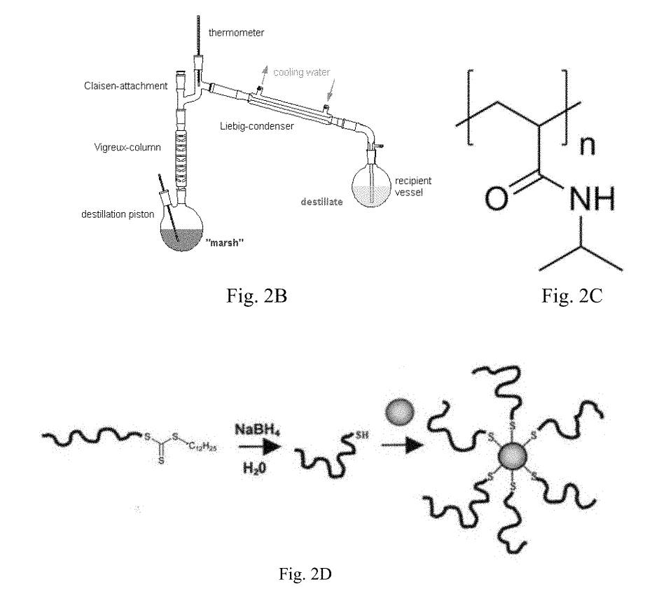

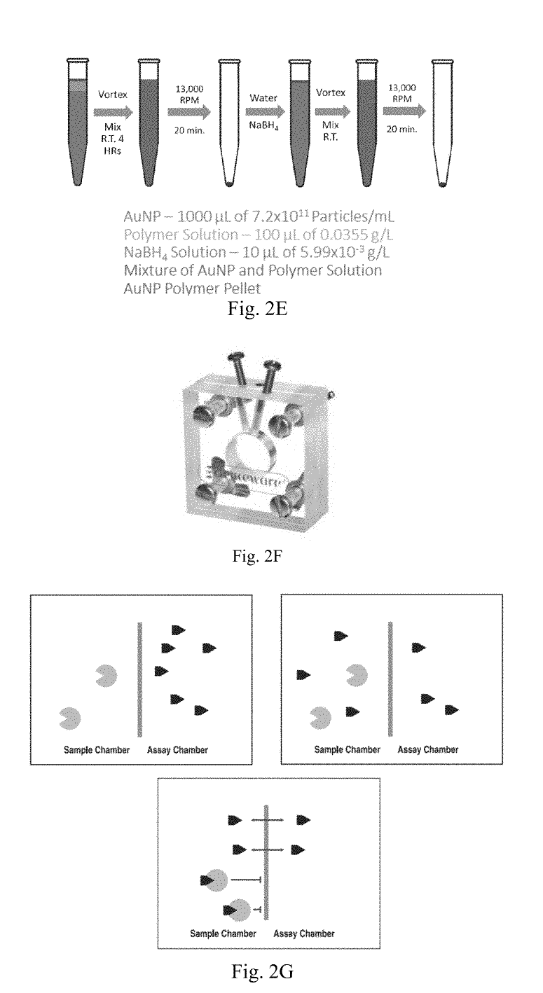

Polymer particle and random coil sizes were measured using a Malvern Zetasizer Nano ZS90 instrument. This instrument uses dynamic light scattering to measure the particle size (FIG. 2A). When polymer is present in a solution, the particles or random coils are suspended in a liquid and are never stationary. Constant movement occurs due to random collisions with the polymer and its surrounding liquid molecules, which is known as Brownian motion. A relationship between the speed of a particle because of Brownian motion and its radius is represented by the Stokes-Einstein equation [Zetasizer Nano User Manual. (2004) Malvern Instruments: Worcestershire, England].

$$D = \frac{{k}_{B} T}{6 π η r}$$

Here, D is the diffusion constant, kB is the Boltzmann constant, T is temperature, η is the viscosity, and r is the molecular radius. The instrument uses a 633 nm red laser to measure the polymer's movement in solution, and as the polymer moves it scatters the light and fluctuates. This constructive and destructive phase addition of the scattered light will cause intensity of brighter and darker areas throughout the cuvette to develop and lessen in their intensity. The Zetasizer Nano will measure these rates of intensity fluctuation. With this data and an algorithm, the size of the particles or random coiled polymers can be calculated. The instrument utilizes the parameters of the solution and contents of the material present to make these calculations. The known viscosity, diffusion coefficient, and refractive index were measured for pure poly (NIPAm) and inserted into the parameters portion of the standard operating procedure for the specific measurement. These values were saved and used throughout the DLS experiments [Mocan, L. et al., (2014) Int. J. of Nanomedicine, 9, 1453; Chu, B., (1970) Ann. Rev. Phys. Chem., 21 (1), 145-174].

To determine the LCST of a given polymer, a temperature trend was applied to the solution and size measurements were taken at the various temperatures. This plot of temperature versus particle size would demonstrate at what temperature the polymer was beginning and fully aggregating in solution. This was done on every polymer in an aqueous solution that was synthesized to determine at what temperature, or temperatures, equilibrium binding dialysis needs to occur. The temperature trend versus polymer size was also done in the presence of the template molecule and other selectivity test molecules to determine whether binding when affects aggregate size [López-Pérez, P. M. et al., (2009) Langmuir, 26 (8), 5934-5941].

### Monomer Purification

All monomers, initiators, and template molecules were purified to remove any unwanted isomers, inhibitors, and excess starting material. All monomers were purchased from Sigma Aldrich for all reported MIP in these experimental embodiments. Additional purification steps were taken.

Solid monomers were recrystallized using the listed solvents above in the reagents section. This was done by heating the various solvents so that the solvent was almost at the boiling point but not boiling. Then the solid monomer was added to the hot solvent and then cooled, very slowly forming crystals. This solvent mixture was vacuum filtered to remove the solid from the solvent (including residual inhibitors) [Tan, N. P. B. et al., (2015) Data in Brief, 5, 434-438]. This was done three times to ensure that all inhibitor and other impurities were fully removed. On the last round of recrystallization, the vacuum filtration was run for about 30 minutes to fully remove all solvent [Roberts, G. E. et al., (2003) J. Polymer Sci. Part A: Polymer Chemistry, 41 (6), 752-765].

All liquid monomers were vacuum distilled to remove the inhibitor, mono methyl ether hydroquinone (MEHQ) and hydroquinone (HQ), present. An apparatus of the vacuum distiller used is shown in FIG. 2B. Once this distillate was done distilling from the marsh they were passed through a column made up of a cotton layer to keep particles out of the final product, inhibitor remover beads, and basic alumina to fully remove all MEHQ and HQ that could be present [Khudyakov, I. V. et al., (1999) Indust. & Engin. Chem. Res., 38 (9), 3353-3359]. This allowed for better distribution and polymer yield to be obtained. Once the polymerization was finalized with purified monomers and a better degassing technique (freeze-pump-thaw), a better binding affinity and selectivity to the template molecule was observed [Hartmann, J. et al., (2006) Macromolecules, 39 (3), 904-907].

### Freeze-Drying Polymer Samples

The first experiment to be completed on any newly synthesized MIP was equilibrium dialysis. Once removal dialysis was complete, the MIP solution was rinsed with deionized water multiple times and then freeze dried using a lyophilizing unit. Reducing the pressure to 0.130 Torr and the temperature lowered to −80° C., below waters triple point, allowed for the removal of water through sublimation, leaving behind a fluffy off white powder of the prepared MIP sample [Dong, A. et al., (1995) J. Pharm. Sci., 84 (4), 415-424]. Then, known concentrations of polymer aqueous solutions were made. This provided knowledge of how much polymer was present during testing and how much template molecule could be theoretically bound to the MIP network.

### Free-Radical Polymerization and Imprinting

Free radical polymerization was first used to develop a polymer that could be used for proof of concept and optimization testing (Table 2A). This was done with the phenol templating experiments to successfully template and selectively bind 4-nitrophenol in a copolymer of NIPAm and functional monomer. This was also used to develop and optimize the polymerization method and polymer formulation. Free radical polymerization is easy and robust enough to make MIPs. The source of radicals used throughout these experiments is AIBN. This is a thermal active source of radicals [Xu, L. et al., (2012) Journal of Hazardous Materials, 233, 48-56].

TABLE 2A
Examples of embodiments of free radical polymerization.

| | | Non- | Recognition | MBAm | |
|---|---|---|---|---|---|
| | NIPAm | Covalent | Monomer (4-VP or | (Covalent | Template |
| Polymers | (Backbone) | Crosslink | MAA) | Crosslink) | (RM:TM) |
| Molecular | Remain % | 5% to | 4% to 15% | 0% to 10% | # of hydrogen biding |
| Imprinted | | 10% | | | sites + 1 extra RM to |
| | | | | | TM) |
| Non- | Remain % | 5% to | 4% to 15% | 0% to 10% | None |
| Imprinted | | 10% | | | |

For the examples in Table 2A, various solvents and amounts were used and are defined throughout the Examples section. AIBN was used as a radical source throughout the experiments in the Examples section. 2% AIBN (by mass) of the overall mass of all monomers present was used to polymerize. In addition, freeze-pump-thaw was completed 3 times and then back filled with nitrogen gas for every polymerization.

### Poly N-Isopropylacrylamide Homopolymer

The first polymer synthesized for this project was a free radical polymerization of n-isopropylacrylamide (NIPAm), a homopolymer, for initial testing and baseline experiments for future comparison (FIG. 2C). A 50 mL round bottom flask had purified NIPAm (25 mmol) 2.825 grams added to it with 25 mL of 1,4-dioxane, 2% (w/w) AIBN, and a stir bar [Schild, H. G., (1992) Prog. in Polymer Sci., 17 (2), 163-249]. The monomer and initiator were dissolved and then sealed with a rubber septum. Next, the polymerization mixture was freeze-pumped-thawed three different times to remove all gas and then back filled with nitrogen, an inert gas, for five minutes. The reaction vessel was then placed into an oil bath at 70° C. for 24 hours with constant stirring. Purification of this polymer was followed as outlined below using dialysis in deionized water as a purification solvent for many cycles.

### Copolymerization of Molecularly Imprinted Polymer

After successful polymerization of poly(NIPAm) the next step was to begin creating MIPs for the desired template molecule, 4-nitrophenol. The addition of several monomers was needed to create a functional MIP. The addition of 4-vinylpryidine (4VP) was important as the recognition, or functional, monomer. Forms of crosslinking through various monomer additions to the polymerization solution were also explored. The main backbone monomer present in all of these MIPs was NIPAm because of its ability to swell and shrink when in an aqueous environment. Many of the polymers prepared were made up of 60-80% NIPAm, 5-10% 4VP in varying ratios to template molecule, and with varying amounts of MBAm and a variety of non-covalent crosslinking combinations, and initiator amounts as described in Example 2. To optimize the MIP overall function, different polymerization solvents were used: buffered water, dimethyl sulfoxide, tetrahydrofuran, acetonitrile, acetone and 1,4-dioxane, in a variety of volumes to change the monomer concentration before initiation. Overall, most factors of the polymerization method were altered and then studied to produce the best selective binding MIP possible. All monomers were purified as stated above in the first section. All of these polymers were freeze-pumped-thawed three times and then back filled with nitrogen for 5 minutes before being placed in an oil bath at 70° C. for 16 to 32 hours with constant stirring. Removal dialysis was done before purification in these MIPs. The removal solution was made up of 70% deionized water, 20% methanol, and 10% glacial acetic acid and changed multiple times over a wide range of solvation times at various temperatures.

### Polymer Purification and Removal of Templated Molecule

After synthesis of any polymer or MIP the polymerized solution was placed into a dialysis bag. Most of the polymers synthesized in these studies were dialyzed using a 10,000 to 12,000 molecular weight cut off (MWCO) dialysis membrane. This dialysis bag was created by securely closing the ends of a length of dialysis tubing with plastic dialysis clips. The dialysis bag was placed into a vessel that has an order of magnitude more volume than the bag itself [Neufeld, C. & Marvel, C., (1966) J. Polymer Sci. Part A-1: Polymer Chemistry, 4 (11), 2907-2908].

The purification solvent was determined based on the polymer and its monomer unit's solubility. This allowed the small chains, unreacted monomers, and template molecule (when present) to be removed while leaving the fully synthesized polymer chains or particles inside the bag. This purification solvent was changed frequently in the beginning of polymer purification and then less frequently at the end. A long dialysis time was used, generally from 4 weeks to 6 months using a 10,000 to 12,000 MWCO dialysis membrane, to allow the polymer to unravel and fully release all small chains and unreacted monomer.

The template removal solvents were used to remove unbound template molecule present in the polymerized solution. It took months to fully remove the entire template. It is believed that many binding sites may be present buried within the polymer. To overcome this, the polymer solution was alternated between acidic and basic conditions, at varying temperatures and durations, to modify the polymers conformation, and expose bound template molecule. Once exposed, template can dissociate from the polymer, creating a binding site. Placing smaller concentrations of polymerized solution in the dialysis bag decreased the time and amount of solvent necessary to completely remove the template molecule.

### Raft Polymerization

Reversible addition-fragmentation chain transfer (RAFT) polymerization process was used to prepare monodisperse MIP. This technique was used once the polymer was fully optimized for the best binding and selectivity towards a specific template molecule. 2-(Dodecylthiocarbonothioylthio)-2-methylpropionic acid (DDMAT) was the RAFT agent used in the polymerizations. Chain length was varied by changing the ratio of RAFT agent to monomer concentration to initiator concentration in the polymerization solvent. The length of the MIP would be very precisely controlled because each monomer unit has a specific size, and by adding a specific ratio of monomer to RAFT agent it was possible to obtain relatively uniform and desired chain length from the polymers [Moad, G. et al., (2000) Polymer International, 49 (9), 993-1001].

RAFT takes longer to prepare polymer because the free radicals present are controlled by the RAFT equilibrium. At any given time, the number of propagating chains is very small. Depending on the number of different monomers and template molecule, mixtures were polymerized for anywhere from 48 hours to six days under nitrogen and at the appropriate temperature. Once discovered, the given polymers were resynthesized and retested to determine if this was a chain length or other theoretical problem. The polymer formulations were distributed exactly to the free radical polymerizations but with the added RAFT agent. The use of RAFT enabled the MIP to be attached to a gold substrate for experiments described below herein.

RAFT polymerization was used until optimization was completed using free radical polymerization. Ratios of RAFT agent to monomer to initiator were successfully explored, however it was realized that a 1000 to 1 to 1 ratio yielded the best MIPs. The increased chain length allowed high molecular weights and longer chains that yielded better binding site formation.

### Attachment of Raft MIP to Gold Nanoparticle

The chosen RAFT agent, DDMAT, is left as a dithioester end group after polymerization. Dithioester end groups can be reduced by sodium borohydride to a thiol end group. In aqueous media at ambient temperature, the thiol reacts with gold nanoparticles (FIG. 2E). These MIP coated gold nanoparticles are produced within hours by the reaction shown in FIG. 2D [Lowe, A. B. et al., (2002) Journal of the American Chemical Society, 124 (39), 11562-11563].

### Spinning Down MIP-Coated Gold Nanoparticles: Binding Capabilities

Gold Nanoparticles (AuNPs) were easily spun down when in an aqueous solution and a binding constant was determined. These AuNPs were spun down and dried so just MIP-coated AuNP was left out of solution. These dried AuNPs were then added to the template molecule solution and given time to equilibrate with temperature and solvate the AuNPs. Then the AuNPs were spun down to a pellet and the supernatant was measured for the presence of the template molecule. The concentration of template molecule present in the supernatant was the amount of unbound template molecule and the difference in the initial concentration of template molecule was the amount bound to the MIP-coated AuNP. This method was carried out at several template concentrations. The template binding capacity and binding affinity constant were calculated from this data as described in Example 4 [Matsui, J. et al., (2005) Analytical Chemistry, 77 (13), 4282-4285].

### Binding Experiments

Binding affinities were measured by equilibrium dialysis. This technique utilized a poly acrylic or high density polyethylene (HDPE) block that consisted of two sides that can be screwed together. Each side had the same fixed volume and size channel. At the top there were two ports that could be accessed after the block was screwed together. The two sides were separated by a cellulose membrane with a specific molecular weight cut off (MWCO) pore size so that polymer is contained to one side of the block and template could pass through the membrane and equilibrate with the other side [Oppenheimer, J. H. et al., (1963) Journal of Clinical Investigation, 42 (11), 1769]. This block could be maintained at a controlled temperature and an example of such a poly (acrylic) block is shown in FIG. 2F [Balis, F. M. et al., (1987) Journal of Clinical Oncology, 5 (2), 202-207]. Binding to the MIP can affect the molar absorptivity or fluorescence efficiency, therefor it was only necessary to measure the molecule side and determine the amount bound by the difference.

The block was given an appropriate amount of time (48 to 120 hours) to fully equilibrate. FIG. 2G shows embodiment of how equilibrium dialysis was set up at the two different equilibrium points. Assuming no binding was taking place, the block should have equal concentrations of template molecule on each side of the block at equilibrium. The distribution ratio would be 1.00. The second point of equilibrium is between the molecule in question being bound or not to the MIP. Increased binding removes “free” molecule from the aqueous solution and drives the first equilibria. With the possibility of binding occurring, the block was given extra time to allow it to re-equilibrate after the binding phenomenon. A distribution ratio, or the amount of bound molecule divided by the amount of free molecule, equal to one represented binding. Greater than one indicated the MIP affinity toward binding the molecule present [Balis, F. M. et al., (1987) Journal of Clinical Oncology, 5 (2), 202-207].

Calculation of the distribution ratios was possible by combining the use of equilibrium dialysis and a calibration curve completed using a spectroscopic technique. By measuring standard concentrations of the template and creating a calibration curve Beer's law, Absorbance=ε L c was employed. Measuring the template side (assay side in FIG. 2G) and calculating the concentration after the equilibration time (3 to 7 days), permitted the concentration present on the MIP side to be inferred. The molar concentration of template present after equilibration, T_{off}, subtracted from the original concentration of template, T_{original}, resulted in the concentration of template present on the MIP side, Tₒₙ. Then the below equation was used to calculate the distribution ratio, KD [Rice, N. et al., (1993) Pure and Applied Chem., 65 (11), 2373-2396].

$${K}_{D} = \frac{{T}_{on}}{{T}_{off}} = \frac{{T}_{original} - {T}_{off}}{{T}_{off}}$$

This calculation was performed for every equilibrium dialysis experiment described in the Examples.

### Example 2

### Optimizing a Selectively Binding Molecularly Imprinted Polymer Network: Analyzed using Equilibrium Dialysis (Methods as Described in Example 1)

### Data and Results

### Poly (NIPAm-CO-4VP) Initial Characterization and Testing

A method to prepare a NIPAm backbone monomer was employed with the recognition monomer of 4-vinylpyridine to imprint 4-nitrophenol [Caro, E. et al., (2002) J. Chromatog. A 963 (1), 169-178]. It was previously shown that 4-VP is a better recognition monomer for nitrophenol template than MAA [Huang, X. et al., (2003) J. Mol. Recognition, 16 (6), 406-411; Herrero-Hernández, E. et al., (2011) Int. J. Mol. Sci. 12 (5), 3322-3339]. 4-nitrophenol was chosen because of the molecule's history as a ground water poison [Fischer, F. et al., (2000) Schriftenreihe des Vereins fur Wasser-, Boden- und Lufthygiene, 107, I-x, 1-108]. In addition, 4-nitrophenol has isomers commonly available that make selectivity measurements possible, and detecting the presence of these compounds was straightforward. Through Uv/vis spectroscopy and deprotonating the molecule, low concentration measurements could be taken (5.0×10⁻⁵ M) [Biggs, A., (1954) Transactions of the Faraday Society, 50, 800-802].

Prior systems, like most MIPs, used extreme amounts of covalent crosslinking monomers [Abdollahi, E. et al., (2015) Polymer Reviews, (just-accepted), 00-00]. Results of some of the present studies showed that use of minimal covalent crosslinking, resulted in measurable binding affinity; however, selectivity was lower than desired. To increase selectivity, experiments were performed to utilize protein-like crosslinking [Friedman, M., (2013) Springer Science & Business Media; Vol. 86].

Prior efforts had included tests with no crosslinking, or very low amounts of crosslinking (10-20%), compared to typical MIP formulations. In addition, various solvents and solvent systems were used as polymerization solvents and removal solvents. There was not a coherent solvent system that specifically worked best when polymerizing these MIPs. Removal dialysis was done with water and a combination of methanol and water. It was not understood how this polymerization took place and low yields were witnessed once lyophilization (freeze drying) of the MIP solutions was completed.

Studies described herein were performed to determine an effective monomer concentration, polymerization solvent, functional monomer to template molecule ratio, and a crosslinking network that could be used to form a high binding affinity MIP while maintaining selectivity. 4-nitrophenol was selected in some studies as the imprinting template because the para position of the hydrogen bonding sites were thought to yield high affinity binding sites [Caro, E. et al., (2003) J. Chromatog. A, 995 (1), 233-238].

All MIPs described in studies in this Example were synthesized using the free radical technique, using methods described elsewhere herein. This allowed for fast polymerization times and quicker turn arounds on whether binding was being exhibited. Instead of in prior studies when a first step included characterization to determine LCST and polymer make up, in the present studies, an initial step was to measure the affinity and selectivity of prepared MIPs at room temperature to determine whether affinity and selectively were present with a given MIP.

Monomer purification was completed on all monomers, as outlined in Example 1. The original MIP 1 used a 50 mmol of total monomer in the feed ratio, 80% NIPAm, 10% 4VP and 10% MBAm, with a 3 to 1 ratio of 4VP to 4N. MIP 1 was polymerized using the free radical technique using AIBN as the initiator in the mass amount of 2% of the total monomer weight (w/w %). Some tested MIP preparation methods utilized one more recognition monomer unit than the number of hydrogen binding sites about the template molecule. For the various experimental conditions that were tested and present in this Example, distribution ratios are reported. These distribution ratios were the factor that determined if the polymer formulation or polymerization system was successful.

### Polymerization Solvent: Effects on Imprinting

Studies were performed to determine an appropriate solvent mixture for use in preparing MIPs. Equilibrium dialysis was used to test an MIP's affinity for the template molecule [van Liempd, S. et al., (2011) Journal of the Association for Laboratory Automation, 16 (1), 56-67]. As the MIP polymerization system changed and was refined, each polymer was tested using this method. An increase in the distribution ratio indicated that the MIP was binding with higher strength. The goal of these studies was not necessarily to develop the polymers' synthetic structure but was focused on the ability of the template to imprint within the polymer structure, forming a high affinity binding site.

Differences in each of these polymers are the listed solvent systems that were present during polymerization. The feed ratio of monomers was used as outlined and then each solvent was added to the reaction vessel until all monomers were sufficiently dissolved. This amount as noted as J.E.D. (just enough to dissolve). Each solvent was used in a similar method unless otherwise stated. This was to ensure that the particular polymerization solvent was being tested and other factors were kept constant.

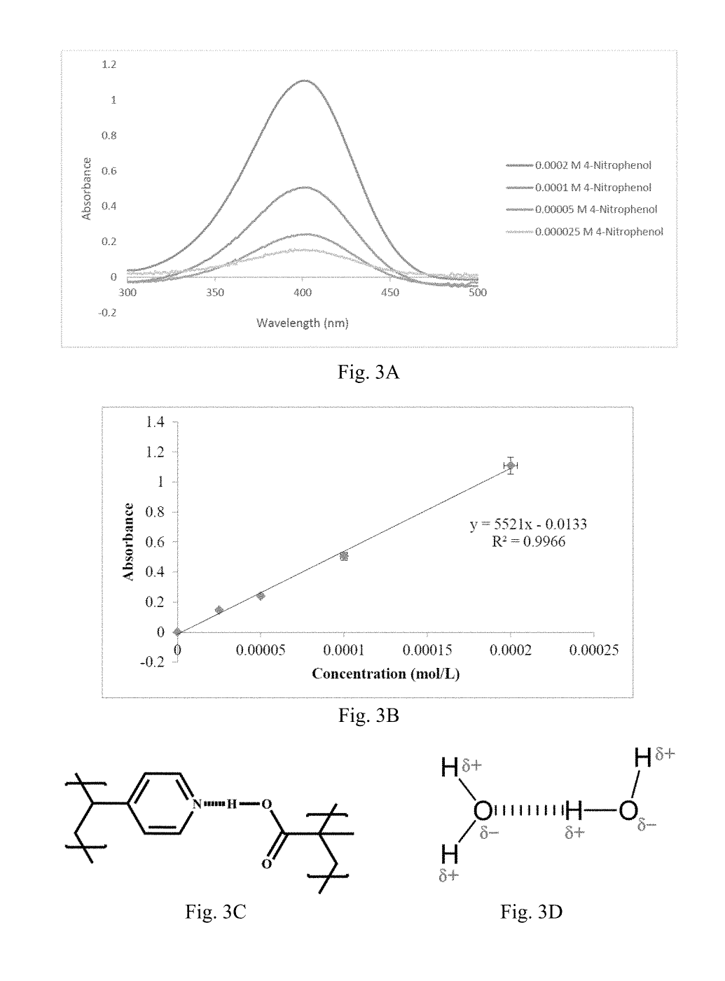

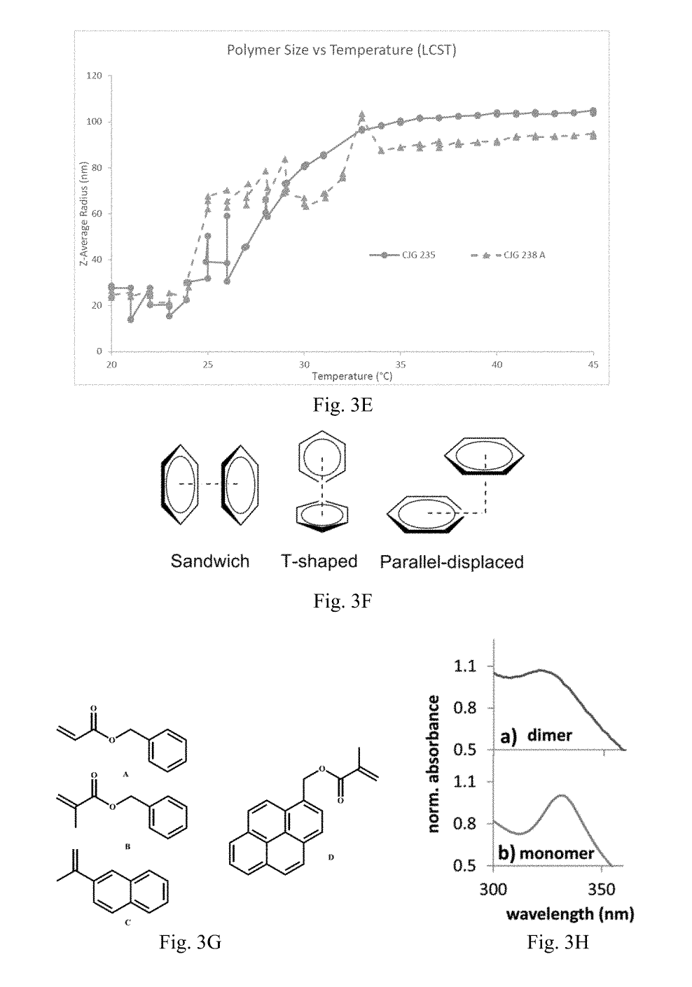

As indicated in Example 1, the distribution ratio was calculated using a spectroscopic technique (FIG. 3A), Uv/vis spectroscopy for 4-nitrophenol and Fluorescence spectroscopy for fluorescein, coupled with Beer's Law and standards plot (FIG. 3B). Concentration of the template present after the experiment was calculated (Table 3B). The entire process is shown in the first set of calculations and the distribution ratios are reported for subsequent experiments [Al-Ahmary, K. M. et al., (2011) J. Mol. Liquids, 158 (3), 161-165; Leonhardt, H. et al., (1971) J. Phys. Chem., 75 (2), 245-249].

### Polymerization in Buffered Water: Effects on Imprinting

Buffered water was utilized to try and form a two-phase solution between the water and the hydrophobic poly (NIPAm) when the temperature was raised above the LCST. It was expected that this would result in the polymer chains aggregating as they were polymerized (Table 3A). It was expected that due to the polarity difference between the monomers, distribution of hydrophilic units in the polymer chains would be favored on the areas contacting the external solvent water [Heskins, M. & Guillet, J. E., (1968) Journal of Macromolecular Science—Chemistry, 2 (8), 1441-1455]. But, as seen in Table 3C, the distribution ratios did not appear to show any binding affinity, even when varied at different MIP and template concentrations.

TABLE 3A
Molecular imprinted polymer formulation with a feed ratio totaling 50 mmol and
polymerization conditions, oil bath for 70° C. for at least 24 hours.

| | | Recognition | MBAm | | | |
|---|---|---|---|---|---|---|
| | NIPAm | Monomer | (Covalent | Template | Polymerization | Free |
| Polymers | (Backbone) | (4-VP) | Crosslink) | (RM:TM) | Solvent | Radical |
| MIP1 | 80% | 10% | 10% | 4- | pH 7 Water | AIBN |
| | | | | Nitrophenol | J.E.D. | 2% |
| | | | | 3:1 | | (w/w) |

TABLE 3B
Concentration of 4-Nitrophenol after equilibrium dialysis experiment
on analyte and polymer side of the dialysis cell. Initial concentration
stated before experiment on the analyte side with all MIP polymerized
in pH 7 water. Equilibrium dialysis experiment were performed at
room temperature.

| MIP 1 | Initial | Absorbance | | POLYMER |
|---|---|---|---|---|
| (g/L) | 4-Nitrophenol | @ 401 nm | ANALYTE (M) | (M) |
| 1 | 2.00 × 10−4 | 0.553 | 9.94 × 10⁻⁰⁵ | 1.01 × 10⁻⁰⁴ |
| 5 | 2.00 × 10−4 | 0.524 | 9.42 × 10⁻⁰⁵ | 1.06 × 10⁻⁰⁴ |
| 10 | 2.00 × 10−4 | 0.511 | 9.18 × 10⁻⁰⁵ | 1.08 × 10⁻⁰⁴ |
| 100 | 2.00 × 10−4 | 0.533 | 9.58 × 10⁻⁰⁵ | 1.04 × 10⁻⁰⁴ |
| 1 | 1.00 × 10−4 | 0.252 | 4.53 × 10⁻⁰⁵ | 5.47 × 10⁻⁰⁵ |
| 5 | 1.00 × 10−4 | 0.239 | 4.30 × 10⁻⁰⁵ | 5.70 × 10⁻⁰⁵ |
| 10 | 1.00 × 10−4 | 0.225 | 4.04 × 10⁻⁰⁵ | 5.96 × 10⁻⁰⁵ |
| 100 | 1.00 × 10−4 | 0.273 | 4.91 × 10⁻⁰⁵ | 5.09 × 10⁻⁰⁵ |
| 1 | 5.00 × 10−5 | 0.136 | 2.44 × 10⁻⁰⁵ | 2.56 × 10⁻⁰⁵ |
| 5 | 5.00 × 10−5 | 0.133 | 2.39 × 10⁻⁰⁵ | 2.61 × 10⁻⁰⁵ |
| 10 | 5.00 × 10−5 | 0.1346 | 2.42 × 10⁻⁰⁵ | 2.58 × 10⁻⁰⁵ |
| 100 | 5.00 × 10−5 | 0.133 | 2.39 × 10⁻⁰⁵ | 2.61 × 10⁻⁰⁵ |

TABLE 3C
Reporting distribution ratios of MIP polymerized in water at varying
concentration of MIP, and template molecule reported in mol/L.
Distribution ratio equal to 1 demonstrates just equilibrium of the
template molecule (4N) throughout the cell. MIP (10 g/L) with (0.1 g/L)
4-nitrophenol yielded the best distribution ratio.

| Polymerization | | | Distribution |
|---|---|---|---|
| Solvent | MIP 1 (g/L) | 4-Nitrophenol (M) | Ratio at ~23° C. |
| ph 7 (Water) | 1 | 2.00 × 10⁻⁴ | 1.01 ± 0.005 |
| ph 7 (Water) | 5 | 2.00 × 10⁻⁴ | 1.12 ± 0.005 |
| ph 7 (Water) | 10 | 2.00 × 10⁻⁴ | 1.18 ± 0.005 |
| ph 7 (Water) | 100 | 2.00 × 10⁻⁴ | 1.09 ± 0.005 |
| ph 7 (Water) | 1 | 1.00 × 10⁻⁴ | 1.21 ± 0.005 |
| ph 7 (Water) | 5 | 1.00 × 10⁻⁴ | 1.33 ± 0.005 |
| ph 7 (Water) | 10 | 1.00 × 10⁻⁴ | 1.47 ± 0.005 |
| ph 7 (Water) | 100 | 1.00 × 10⁻⁴ | 1.04 ± 0.005 |
| ph 7 (Water) | 1 | 5.00 × 10⁻⁵ | 1.05 ± 0.005 |
| ph 7 (Water) | 5 | 5.00 × 10⁻⁵ | 1.09 ± 0.005 |
| ph 7 (Water) | 10 | 5.00 × 10⁻⁵ | 1.07 ± 0.005 |
| ph 7 (Water) | 100 | 5.00 × 10⁻⁵ | 1.09 ± 0.005 |

When looking at the reaction flask during polymerization at 70° C. the solution quickly became cloudy. The NIPAm was becoming hydrophobic very rapidly and polymerization was not able to take place because of aggregation of the NIPAm monomer units. A two-phase solution would allow the NIPAm to be favored in the hydrophobic regions and to add to the chain, but the co-monomers would be favored if the chain was growing in a hydrophilic region [Berezkin, A. V. et al., (2004) New J. of Physics, 6 (1), 44]. This polymer (Table 3A) would exist in a thermodynamically stable configuration where hydrophobic interactions stabilize the interior of the globule while hydrogen bonding stabilizes the surface of the polymer. This form of stabilization is seen and exhibited in the folding of proteins [Dobson, C. M., (2003) Nature, 426 (6968), 884-890].

### Polymerization in a Two-Solvent System: Imprinting Effects

The use of a solvent mixture, water and dimethyl sulfoxide (DMSO) was thought to result in the desired effect and DMSO has been shown to be compatible with an aqueous environment [Schild, H. G. et al., (1991) Macromolecules, 24 (4), 948-952]. A series of polymerizations were completed with varying amounts of pH 7 water (10, 25, 50, 75, and 100%) to determine if this mixture would help form the binding sites with stronger affinity towards 4-nitrophenol (Table 3D). It was hypothesized that the DMSO might act like a porogen because it also was miscible in water [Prasad, B. B. et al., (2010) Talanta, 81 (1), 187-196]. These pockets, or organic pores, would allow the hydrophobic chain ends to form and create a more organized binding site [Chianella, I. et al., (2003) Biosensors and Bioelectronics, 18 (2-3), 119-127]. When polymerization was completed all solutions looked very similar: off white in color and lightly cloudy.

TABLE 3D
Molecular imprinted polymer formulation with a feed ratio totaling 50 mmol and
polymerization conditions, oil bath for 70° C. for at least 24 hours.

| | | Recognition | MBAm | | | |
|---|---|---|---|---|---|---|
| | NIPAm | Monomer (4- | (Covalent | Template | Polymerization | Free |
| Polymers | (Backbone) | VP) | Crosslink) | (RM:TM) | Solvent | Radical |
| MIP 1 | 80% | 10% | 10% | 4- | Varied | AIBN |
| | | | | Nitrophenol 3 | J.E.D. | 2% |
| | | | | to 1 | | (w/w) |

Shown in Table 3E, the MIP formed in pure DMSO yielded the best results. The polymerization took place at 70° C. Because of this, the solvent system with more water present made the NIPAm monomer hydrophobic in this environment, resulting in less NIPAm being polymerized. It was hypothesized that the MIP is made up of very little NIPAm and mostly just the 4VP and MBAm, most of which is a typical MIP formulation with just recognition monomer and crosslinking agent [Wang, X. et al., (2013) Journal of Polymer Science Part A: Polymer Chemistry, 51 (10), 2188-2198]. This is the opposite of the goal for the MIPs being prepared in the present studies [Gao, X. et al., (2013) Int. J. Nanomanufacturing, 9 (3-4), 347-358]. As DMSO was increased in volume, binding increased at the same rate, Table 3E.

TABLE 3E
Distribution ratios of varying amounts of buffered water (pH 7) and
DMSO as a two-part solvent system. Rising distribution ratios
were noted as DMSO rises in percentage of the solvent present.
Pure DMSO had the highest distribution ratio.

| Polymerization | MIP 1 | 4-Nitrophenol | Distribution Ratio |
|---|---|---|---|
| Solvent | (g/L) | (M) | at 23° C. |
| 100% pH 7 Water | 7.062 | 1.00 × 10⁻⁴ | 1.39 ± 0.0072 |
| 90% pH 7/10% DMSO | 7.062 | 1.00 × 10⁻⁴ | 1.41 ± 0.0072 |
| 75% pH7/25% DMSO | 7.062 | 1.00 × 10⁻⁴ | 1.44 ± 0.0072 |
| 50% pH7/50% DMSO | 7.062 | 1.00 × 10⁻⁴ | 1.50 ± 0.0072 |
| 25% pH7/75% DMSO | 7.062 | 1.00 × 10⁻⁴ | 1.56 ± 0.0072 |
| 10% pH7/90% DMSO | 7.062 | 1.00 × 10⁻⁴ | 1.61 ± 0.0072 |
| 100% DMSO | 7.062 | 1.00 × 10⁻⁴ | 1.65 ± 0.0072 |

DMSO was used as the polymerization solvent for a period of time until it was identified that distribution ratios had not increased with further optimization. The polymerization solvent was reexamined after this trend was noticed. The literature provided conflicting indications relating to selection of solvents for this type of MIP network because lightly crosslinked MIPs have not been investigated in detail. Experiments were performed to identify and optimize a solvent system for use in preparing MIPs of the invention.

### Polymerization in Porogenic Solvents: Imprinting Effects

Various porogenic solvents were selected and investigated. 1,4-Dioxane, tetrahydrofuran (THF), acetonitrile, and acetone are all porogenic solvents that were used to synthesize the MIP (Table 3F). Porogenic solvents were used to achieve a pore structure of sufficient permeabilarity. Polarity of the solvent can compete with the template monomer interaction and reduce the binding affinity of the polymer for the template.

TABLE 3F
Molecular imprinted polymer formulation with a feed ratio totaling 50 mmol and
polymerization conditions, oil bath for 70° C. for at least 24 hours.

| | | Recognition | MBAm | | | |
|---|---|---|---|---|---|---|
| | NIPAm | Monomer (4- | (Covalent | Template | Polymerization | Free |
| Polymers | (Backbone) | VP) | Crosslink) | (RM:TM) | Solvent | Radical |
| MIP 1 | 80% | 10% | 10% | 4- | Varied J.E.D. | AIBN |
| | | | | Nitrophenol 3 | | 2% |
| | | | | to 1 | | (w/w) |

With the completion of polymerization and removal of the template molecule, binding affinity was tested for all of these polymers. As shown in Table 3G, 1,4-dioxane yielded a distribution ratio that was twice as high as any other distribution ratio reported to date. The other solvents also demonstrated strong binding affinities in these studies; however, the dioxane value was notably higher. Dioxane is insoluble in water and does not exhibit the same properties of water. Previous conditions of the polymerization tried to mimic an aqueous environment because that is the setting of the MIP [Vasapollo, G. et al., (2011) Int. J. Mol. Sci. 2011, 12 (9), 5908-5945]. 1,4-Dioxane is a non-polar solvent and experiments performed using this type of solvent showed that it increased the binding site formation and facilitated the necessary polar non-covalent interactions.

TABLE 3G
Distribution ratios of various solvents at room temperature.

| Polymerization | | | Distribution |
|---|---|---|---|
| Solvent | MIP 1 (g/L) | 4-Nitrophenol (M) | Ratio at 23° C. |
| 1,4-Dioxane | 10 | 1.00 × 10⁻⁴ | 4.26 ± 0.008 |
| Tetrahydrofuran | 10 | 1.00 × 10⁻⁴ | 2.24 ± 0.048 |
| Acetonitrile | 10 | 1.00 × 10⁻⁴ | 2.78 ± 0.021 |
| Acetone | 10 | 1.00 × 10⁻⁴ | 2.19 ± 0.005 |
| Water | 10 | 1.00 × 10⁻⁴ | 1.59 ± 0.003 |

When comparing the solvents being used throughout this Example, dioxane was the only non-polar porogenic solvent to be used as the polymerization solvent. THF, acetonitrile, acetone, and DMSO are polar aprotic solvents and water is a polar protic solvent. Polar protic solvents are able to hydrogen bond with the surrounding molecules [Brewster, R. E. & Shuker, S. B., (2002) J. Am. Chem. Soc., 124 (27), 7902-7903]. Polar aprotic solvents lack the acidic hydrogen and therefore cannot be hydrogen bond donors but they can accept the hydrogen bonds [Joris, L. et al., (1972) J. Am. Chem. Soc., 94 (10), 3438-3442; Parker, A. J., (1969) Chemical Reviews, 69 (1), 1-32]. 1,4-dioxane was used throughout further experiments presented herein and was used in comparisons of results of the additional optimization changes and additions to methods of preparing the MIP network.

### Monomer Concentration in Feed Ratio: Effects on Binding

After determining a more optimal polymerization solvent to improve the synthesis of the binding cavity, studies were performed to assess whether the polymer sample “gelled out”, meaning did the MIP, during polymerization, not have enough solvent present and form a solid gel like material. The studies were performed to assess whether the polymer formed intra or intermolecular crosslinks throughout the network. Theoretically, if the monomer concentration is decreased, once polymerization takes place chains will form and be further apart. Therefore, when crosslinking monomer is added the crosslinking monomers will add within the chain and not to another chain or network of chains. This may affect how binding occurs within the MIP (Table 3H). The amount of polymerization solvent, 1,4-dioxane, was adjusted and using 25 mmol of total monomer in the feed ratio with 10, 25, 50, 75 and a 100 mL, allowed concentration to be adjusted.

TABLE 3H
Molecular imprinted polymer formulation with a feed ratio totaling 25 mmol and
polymerization conditions, oil bath for 70° C. for at least 24 hours.

| | | Recognition | MBAm | | | |
|---|---|---|---|---|---|---|
| | NIPAm | Monomer (4- | (Covalent | Template | Polymerization | |
| Polymers | (Backbone) | VP) | Crosslink) | (RM:TM) | Solvent | Radical |
| MIP 1 | 80% | 10% | 10% | 4- | 1,4-Dioxane | AIBN |
| | | | | Nitrophenol | Volume Varied | 2% |
| | | | | 3 to 1 | | (w/w) |

TABLE 3I
Distribution ratio varying monomer concentration at room temperature.

| | | 4-Nitrophenol | Distribution |
|---|---|---|---|
| Polymerization Solvent | MIP 1 (g/L) | (M) | Ratio at 23° C. |
| 1,4-Dioxane (10 mL) | 10 | 1.00 × 10⁻⁴ | 3.01 ± 0.001 |
| 1,4-Dioxane (25 mL) | 10 | 1.00 × 10⁻⁴ | 3.51 ± 0.002 |
| 1,4-Dioxane (50 mL) | 10 | 1.00 × 10⁻⁴ | 4.88 ± 0.004 |
| 1,4-Dioxane (75 mL) | 10 | 1.00 × 10⁻⁴ | 4.89 ± 0.002 |
| 1,4-Dioxane (100 mL) | 10 | 1.00 × 10⁻⁴ | 4.92 ± 0.001 |

Visually, it was seen that the MIP gelled out even when there was 10 mL and 25 mL of 1,4-dioxane present. The rest of the volumes were present in a liquid at room temperature. The distribution ratios are presented in Table 3I. As the monomer concentration was decreased the distribution ratio increased, and appeared to level off once at lower concentrations. Based on these experimental findings, later polymerizations included enough solvent so the polymer did not gel out.

### Covalent Crosslinking Affecting how MIP Form in Solution

Absence and presence of low amounts of covalent crosslinking had been the only crosslinking systems investigated. Low amounts ranged from 5-15% of the molar concentration in the feed ratio. With the higher amounts, >7%, polymer settling would occur given enough time, about 10 minutes. These types of MIPs are not considered hydrogels because they are not stable in an aqueous environment and presented as a mixture. However, when ≦5% of a covalent crosslinker was added, the binding affinity of the MIP decreased significantly. Additional testing was performed to assess covalent crosslinking.

Experiments were completed with varying amounts of covalent crosslinking monomer, MBAm, from 1% to 7% of the feed ratio. Experiments were carried out to test amounts of crosslinking for use in the feed ratio. Tested amounts were: 7%, 6%, 5%, 4%, 3%, 2% and 1%. Polymer solids started to form around 6% and definitely at 7% when at room temperature. All polymer solutions were at 10 g/L. These studies were carried out to examine the upper limit of covalent crosslinking that could be added but results indicated that the polymer was still present as a liquid and not as a particle below the LCST. The upper limit of covalent crosslinking in the feed ratio was determined to be 5%. This amount of covalent crosslinker was polymerized several different times to ensure that the polymer would stay in solution and act as a hydrogel.

### Forming Non-Covalent Crosslinking: Binding Affinity of MIP

Proteins use non-covalent crosslinks and they needed to be incorporated into the MIP network if high affinity binding was to occur. Prior studies have shown that it is possible to achieve this with hydrophobic interactions. Experiments were performed to assess and determine methods to produce MIPs that mimic how proteins form their crosslinks. Non-covalent interactions are the dominant type of interaction between super molecules, such as proteins and DNA. A less rigid bond, created by dispersing a type of electromagnetic interaction between molecules, was examined to determine if it would allow the MIP to be in a hydrogel state; thus, the MIP could be present as a liquid when in an aqueous environment. Experiments were performed to assess the use of different types of types of non-covalent interactions in order to prepare this type of polymer: π-π stacking, ionic, and acid base interactions. These interactions represent major classes of non-covalent bonds and were possible to use within the aqueous system. The use of the MIPs semi-hydrophobic state at the LCST was also utilized and described in detail elsewhere herein (see Example 3).

### Acid-Base Crosslinking

An initial experiment was performed to assess the use of an acid and a base monomer unit to form acid-base crosslinks. This would be a formation of hydrogen bonding similar to the binding site formation. The base unit, 4-vinylpryidine, would hydrogen bond to the OH group of the acid unit, methacrylic acid, see FIG. 3C, forming a dipole-dipole interaction that involves the partially positive hydrogen atom and a highly electronegative, partially negative nitrogen atom. This bond is very strong, similar to a covalent bond; however, it is classified as a very strong dipole-dipole (non-covalent) interaction. This type of bond is the reason that water is a liquid at room temperature, FIG. 3D. The strength of hydrogen bonding in this interaction has been known to be as durable as 40 kcal/mol.

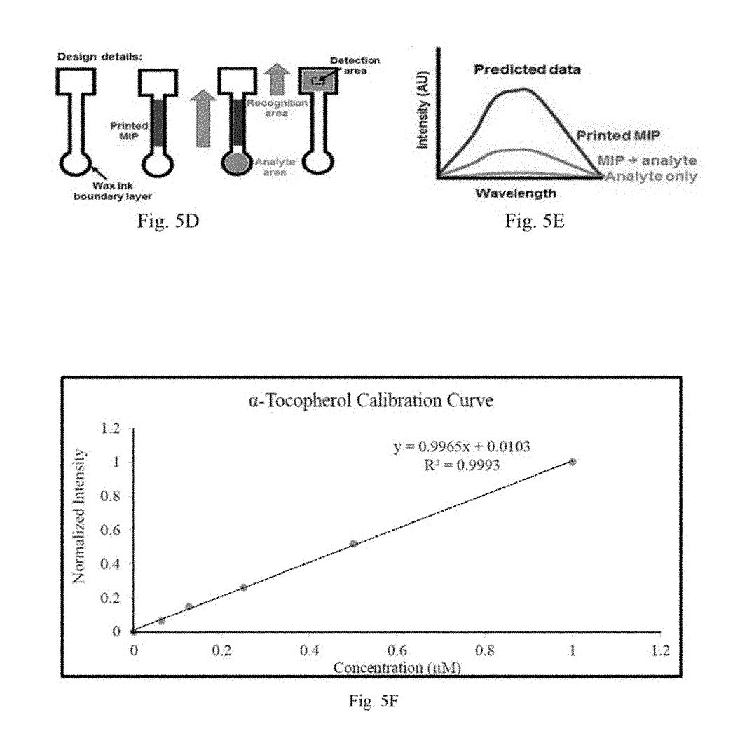

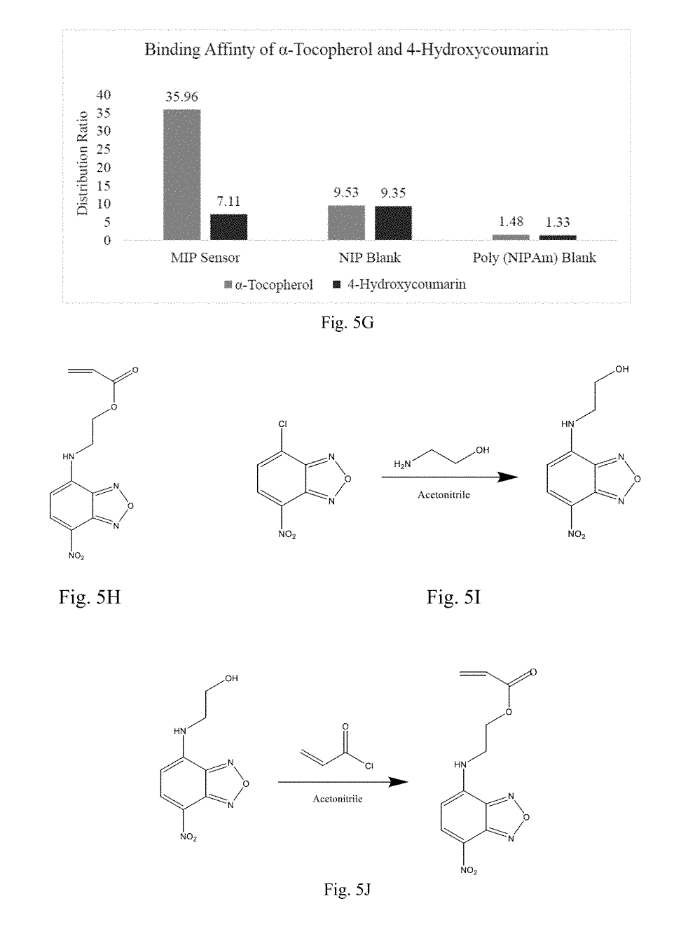

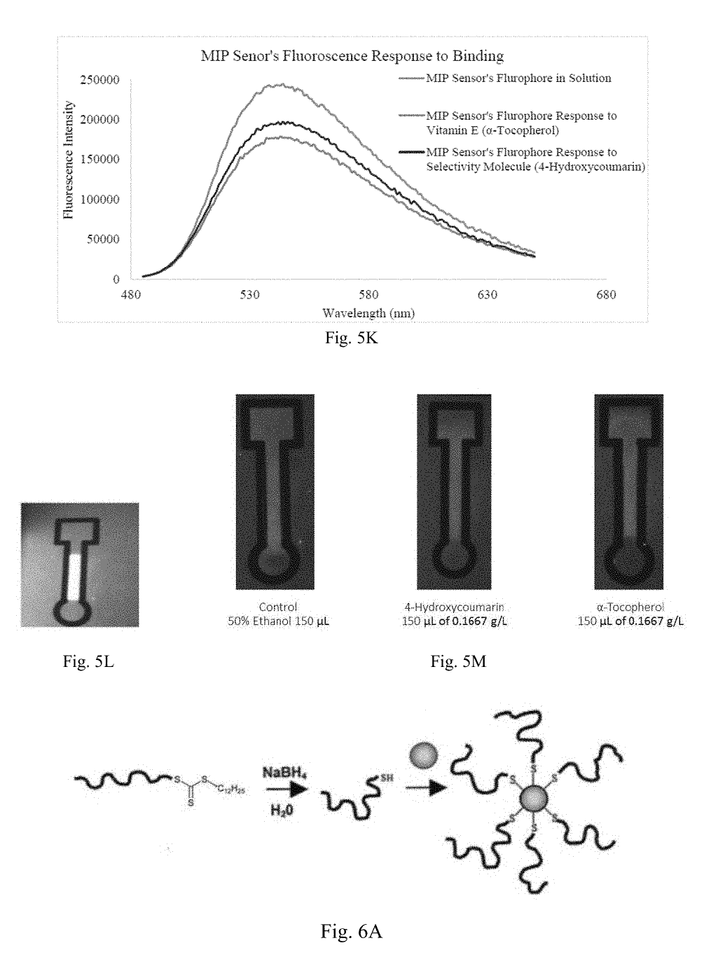

In experimental embodiments, the polymerization incorporated the monomer units: methacrylic acid and 40-vinylpryidine as recognition monomers, in combination with NIPAm as the main backbone unit. A free radical polymerization that imprinted 4-nitrophenol, using 90% NIPAm, 5% MAA, and 5% 4VP was synthesized (Table 5J: CJG 235). The competition of the recognition monomer (4VP) was perceived theoretically. The 4-vinylpryidine had two functions in this MIP, one to form hydrogen bonds with the MAA, crosslinking the MIP, and the other to form hydrogen bonds with the template molecule, forming the binding site. Therefore, calculated amounts of 4VP were used within the feed ratio so enough monomer units were present to complete both goals. Another free radical polymerization imprinted with 4-nitrophenol using 85% NIPAm, 5% MAA, and 10% 4VP was synthesized (Table 3J: CJG 238A). These results can be seen in the DLS spectra (FIG. 5E), identifying the LCST of the various polymer, and the equilibrium dialysis experiments resulting in the distribution ratios of the MIP interacting with the template molecule, 4-nitrophenol.

TABLE 3J
Molecular imprinted polymer formulation with a feed ratio totaling 50 mmol and
polymerization conditions, oil bath for 70° C. for at least 24 hours.

| | | Recognition and | Acid | | | |
|---|---|---|---|---|---|---|
| | NIPAm | Base Monomer | Monomer | Template | | Free |
| MIP | (Backbone) | (4-VP) | (MAA) | (RM:TM) | Solvent | Radical |
| CJG | 90% | 5% | 5% | 4- | 1,4- | AIBN |
| 235 | | | | Nitrophenol 3 | Dioxane | 2% |
| | | | | to 1 | 100 mL | (w/w) |
| CJG | 85% | 10% | 5% | 4- | 1,4- | AIBN |
| 238 A | | | | Nitrophenol 3 | Dioxane | 2% |
| | | | | to 1 | 100 mL | (w/w) |

TABLE 3K
Distribution ratios for binding affinity and selectivity of the MIPs at room
temperature.

| | | | Distribution |
|---|---|---|---|
| MIP (10 g/L) | Analyte | Concentration (mol/L) | Ratio at 23° C. |
| CJG 235 | 2-Nitrophenol | 1.667 × 10⁻⁴ | 0.996 ± 0.0058 |
| CJG 235 | 3-Nitrophenol | 1.667 × 10⁻⁴ | 1.055 ± 0.0058 |
| CJG 235 | 4-Nitrophenol | 1.667 × 10⁻⁴ | 1.243 ± 0.0058 |
| CJG 238A | 2-Nitrophenol | 1.667 × 10⁻⁴ | 1.321 ± 0.0058 |
| CJG 238A | 3-Nitrophenol | 1.667 × 10⁻⁴ | 1.461 ± 0.0058 |
| CJG 238A | 4-Nitrophenol | 1.667 × 10⁻⁴ | 2.870 ± 0.0058 |

These MIPs were both present as a liquid after polymerization and no settling was observed during the six-week removal process. The MIP was present as a liquid once it was rehydrated after lyophilization. There was a great phase transition between 30° C. and 34° C. The MIP started to aggregate around 25° C., transitioning from a random coil to a globule, and once the temperature was increased to 32° C. theses globules aggregated together and formed larger polymer particles. Results from both MIPs showed that they had an LCST value around 28° C., because of the increased amounts of 4VP the LCST value is lowered from 32° C. of pure poly (NIPAm).

Binding was drastically increased when the excess 4VP was added to the network so there was no competing 4VP for binding site construction or crosslinking function. However, as binding of 4-nitrophenol was increased with CJG 238 A, so was the binding of the isomers. These results indicated that this MIP was not very selective (Table 3K). Nevertheless, non-competing amounts of 4-vinylpryidine in combination of acid-base crosslinks demonstrated that this method can be used as a non-covalent crosslink. The amount of acid-base crosslinks was increased throughout the MIP to determine whether more non-covalent crosslinks would help stabilize the binding site (Table 3L). The acid-base crosslinks were increased from 10% to 20% total acid-base crosslinks, and the resulting MIP was called CJG 258 B.

TABLE 3L
Molecular imprinted polymer formulation with a feed ratio totaling 50 mmol and
polymerization conditions, oil bath for 70° C. for at least 24 hours.

| | | Recognition and | Acid | | | |
|---|---|---|---|---|---|---|
| | NIPAm | Base Monomer | Monomer | Template | | Free |
| MIP | (Backbone) | (4-VP) | (MAA) | (RM:TM) | Solvent | Radical |
| CJG | 75% | 15% | 10% | 4- | 1,4- | AIBN |
| 258 B | | | | Nitrophenol | Dioxane | 2% |
| | | | | 3 to 1 | 100 mL | (w/w) |

TABLE 3M
Binding and selectivity results of MIP CJG 258B done
at room temperature.

| | | | Distribution |
|---|---|---|---|
| MIP (10 g/L) | Analyte | Concentration (M) | Ratio at 23° C. |
| CJG 258 B | 2-Nitrophenol | 1.667 × 10⁻⁴ | 2.54 ± 0.001 |
| CJG 258 B | 3-Nitrophenol | 1.667 × 10⁻⁴ | 2.59 ± 0.001 |
| CJG 258 B | 4-Nitrophenol | 1.667 × 10⁻⁴ | 6.61 ± 0.001 |

Increasing the acid-base crosslinking increased the binding of the template molecule two fold. However, selectivity of the MIP was still not improving with the increase (Table 3M). When removing the template molecule, drastic shifts between acidic and basic solutions is needed to swell and shrink the polymer. Detangling the polymer chains allows removal of the template. The same crosslinking monomer units coupled during polymerization were likely not being recoupled because of the pH changes. Consequently, a second form of non-covalent crosslinking was added within the polymer network to try and hold the same acid-base units within the vicinity. As in proteins, it was expected that the use of two or more non-covalent crosslinks would result in MIPs with a high affinity binding site while still maintain the MIP as a liquid.

### π-π Stacking Crosslinking

π-π Stacking refers to the attractive, noncovalent, interaction between aromatic rings. These types of compounds contain it bonds, hence the name, and there is an alignment of positive electrostatic potential on one ring with the negative of another ring forming an offset stack or T-shaped stack. In MIP networks under examination in the study, the affinity of different aromatic monomers to form a dimer could form a crosslink. FIG. 3F shows three versions of benzene dimer formation.

Four different monomer units were tried in experiments performed to obtain a dimer or π-π stacking crosslink, benzyl acrylate (A), benzyl methacrylate (B), 2-isopropenylnathalene (C) and 1-Pyrenemethyl Methacrylate (D) shown in FIG. 3G. Benzyl acrylate and benzyl methacrylate were used in a similar capacity. The only difference was the methyl group added on the backbone of the polymer. Methyl groups next to the vinyl group allowed for higher yield of polymer because the reactivity ratios are more suitable with the methyl version of the monomer. 2-Isopropenylnathalene and 1-pyrenemethyl methacrylate were thought to obtain a strong π-π stacking interaction because more it bonds are present. Three separate polymerizations were prepared by adding 10% of benzyl methacrylate, 10% benzyl methacrylate and 10% 2-isopropenylnathalene, and 10% benzyl methacrylate and 10% 1-pyrenemethyl methacrylate, to the feed ratio of the 20% acid-base and NIPAm backbone MIP. These MIPs were called CJG 276 A, CJG 276 B, and CJG 278 C respectively (Table 3N).

These MIPs varied from previous MIPs because less NIPAm was incorporated. The following results in Table 3O show that the addition of the n-n stacking interactions yields almost similar results to the acid-base non-covalent crosslinking alone. Consistently showing each of these non-covalent interactions, while present or not, were not holding the binding site to selectively sense.

TABLE 3N
Molecular imprinted polymer formulation with a feed ratio totaling 50 mmol and
polymerization conditions, oil bath for 70° C. for at least 24 hours.

| | | Recognition | | | | | |
|---|---|---|---|---|---|---|---|
| | | Base | Acid | π-π | | | |
| | | Monomer | Monomer | Stacking | Template | | Free |
| MIP | NIPAm | (4-VP) | (MAA) | monomer | (RM:TM) | Solvent | Radical |
| CJG | 65% | 15% | 10% | 10% | 4- | 1,4- | AIBN |
| 276 A | | | | BMA | Nitrophenol | Dioxane | 2% |
| | | | | | 3 to 1 | 100 mL | (w/w) |
| CJG | 65% | 15% | 10% | 10% | 4- | 1,4- | AIBN |
| 276 B | | | | 2IPN | Nitrophenol | Dioxane | 2% |
| | | | | | 3 to 1 | 100 mL | (w/w) |
| CJG | 65% | 15% | 10% | 5% | 4- | 1,4- | AIBN |
| 276 C | | | | BMA | Nitrophenol | Dioxane | 2% |
| | | | | 5% | 3 to 1 | 100 mL | (w/w) |
| | | | | 2IPN | | | |

TABLE 3O
Distribution ratios with the template and its isomer for the π-π stacking
crosslinked MIPs completed at room temperature.

| | | | Distribution |
|---|---|---|---|
| MIP (10 g/L) | Analyte | Concentration (M) | Ratio at 23° C. |
| CJG 276 A | 3-Nitrophenol | 1.667 × 10⁻⁴ | 3.54 ± 0.002 |
| CJG 276 A | 4-Nitrophenol | 1.667 × 10⁻⁴ | 7.34 ± 0.002 |
| CJG 276 B | 3-Nitrophenol | 1.667 × 10⁻⁴ | 2.54 ± 0.002 |
| CJG 276 B | 4-Nitrophenol | 1.667 × 10⁻⁴ | 6.86 ± 0.002 |
| CJG 278 C | 3-Nitrophenol | 1.667 × 10⁻⁴ | 3.42 ± 0.002 |
| CJG 278 C | 4-Nitrophenol | 1.667 × 10⁻⁴ | 4.34 ± 0.002 |

It was identified that there were 4 to 5 different monomers present in the feed ratio. The reactivity ratios between each monomer unit are lowered by each unit reacting to form the polymer. It was difficult to match all of these reactivity ratios, therefore yielding low amounts of polymer. In addition, the dimer formation of the various monomer units in these studies did not display a dimer shift when looking at the UV-visible spectra when only the monomer and polymer were placed in solution by themselves. FIG. 3H provides an example of what would be seen if a benzyl methacrylate dimer was to be observed (A) and what the monomer spectra looks like (B). This was observed with all other monomer units at their particular absorbance wavelength and no shifts indicating a dimer were present.

A polymerization of the acid-base crosslinks and addition of ionic crosslinks was done at low amounts, keeping the NIPAm monomer>80% of the feed ratio. The goal was still to find a non-covalent system that can be used to form crosslinks that would enable the binding site to be held together to selectively bind the template molecule, and in addition, be able to withstand the template removal process and interacting with the exact same opposing monomer unit as formed during polymerization.

### Ionic Crosslinking

Ionic crosslinking would involve the attraction of ions from desired monomer units that would undergo full permanent charges of opposite signs. 2-Acrylamido-2-Methyl-1-Propanesulfonic Acid Sodium Salt would represent the positive ion monomer and [3-(Methacryloylamino)propyl] Trimethylammonium Chloride is representative of the negative ion monomer. These interactions have been shown to be harder to break than covalent bonds. Electrostatic interaction between the oppositely charged ions is strong and known as electrovalence [Lien, S.-M.; et al., (2008) Mat. Sci. and Eng.: C 28 (1), 36-43].

Experiments were performed using less crosslinking and recognition monomers, a strategy that improved binding and selectivity in other systems [Sharma, P. S. et al., (2015) Electrochemistry Communications, 50, 81-87]. Experiments were run that produced the polymerization of CJG 286 B: 82% NIPAm, 9% 4VP, 5% MAA, 2% AMPS, and 2% MPTA imprinted with 4-nitrophenol polymerized by free radical (Table 3P). After polymerization, the mixture was slightly turbid but still a liquid at room temperature and completely aqueous after removal dialysis. A goal of these studies included preparing a sensing system in an aqueous environment that permits the swelling and shrinking function of poly (NIPAm) to be exploited. In contrast, ionic bonds are easily broken when present in a polar solvent, especially water [Ostrowska-Czubenko, J. & Gierszewska-Drużyńska, M., (2009) Carbohydrate Polymers, 77 (3), 590-598].

TABLE 3P
Molecular imprinted polymer formulation with a feed ratio totaling 50 mmol and
polymerization conditions, oil bath for 70° C. for at least 24 hours.

| | | Recognition | | | | | |
|---|---|---|---|---|---|---|---|
| | | and Base | Acid | Ionic | | | |
| | | Monomer | Monomer | Crosslinks | Template | | Free |
| MIP | NIPAm | (4-VP) | (MAA) | Monomers | (RM:TM) | Solvent | Radical |
| CJG | 82% | 9% | 5% | 2% | 4- | 1,4- | AIBN |
| 286 B | | | | MPTA | Nitrophenol | Dioxane | 2% |
| | | | | 2% | 3 to 1 | 100 mL | (w/w) |
| | | | | AMPS | | | |

TABLE 3Q
Distribution ratios of ionic crosslinked MIPs.

| | | | Distribution |
|---|---|---|---|
| MIP (10 g/L) | Analyte | Concentration (M) | Ratio at 23° C. |
| CJG 286 B | 2-Nitrophenol | 1.667 × 10⁻⁴ | 1.33 ± 0.0058 |
| CJG 286 B | 3-Nitrophenol | 1.667 × 10⁻⁴ | 1.43 ± 0.0058 |
| CJG 286 B | 4-Nitrophenol | 1.667 × 10⁻⁴ | 2.66 ± 0.0058 |

The crosslinks formed by the acid-base monomer units allowed the binding site to be slightly formed, but the ionic crosslinks were not held together at all in an aqueous solution. Results of the binding experiments supported this theory (Table 3Q). It is possible that these ionic bonds can be formed in a non-polar solvent, like 1,4-dioxane, and that the ionic bonds may very well be formed in the polymerization solvent and be stronger than the typical covalent crosslinks.

### Combining Non-Covalent and Covalent Crosslinking

Studies performed using two forms of non-covalent bonds to prepare a MIP indicated that the approach was not successful in producing an MIP that bound the template molecule selectivity. Studies performed to assess the formation and strength of the acid-base interactions supported their use to form non-covalent crosslinks to form the binding complex. The use of low covalent crosslinked polymers resulted in slightly insoluble particle gels. Combining ultra-low covalent crosslinking (<5%) with non-covalent crosslinking acid-base interactions to make up crosslinking network provided a useful MIP solution.

Studies were performed in which two different amounts of total acid-base monomer were added to the feed ratio, 20% and 10%, with excess 4-vinylpryidine so there was 4% remaining to freely act as the recognition monomer (Table 3R). These amounts were chosen to compare with the values previously polymerized to create a MIP. It was also determined that using less of the MIP would enable it to detangle faster and not be folded up on itself, covering potential binding sites. Using less of the MIP in binding experiments meant that less template molecule had to be used. Therefore, the template molecule was changed from 4-nitrophenol to fluorescein. Fluorescein is a known fluorescence compound and can be detected by fluorescence spectroscopy at nano-molar levels. The ability to detect at such low levels permitted additional binding and kinetics experiments to be performed.

Switching from 4-nitrophenol to fluorescein changed the ratio of recognition monomer to template molecule because fluorescein has one more hydrogen bonding site present than 4-nitrophenol, changing the ratio from 3 to 1 recognition to template to 4 to 1. This was reflected in the amount of template molecule added to the pre-polymerization solution. The removal process of the template molecule was significantly longer than was the case in the 4-nitrophenol studies. It previously took 4 to 6 weeks to remove the nitrophenol template; however, it took upwards of 6 months to remove fluorescein.

TABLE 3R
Molecular imprinted polymer formulation with a feed ratio totaling 50 mmol and
polymerization conditions, oil bath for 70° C. for at least 24 hours.

| | | Recognition | | | | | |
|---|---|---|---|---|---|---|---|
| | | and Base | Acid | MBAm | | | |
| | | Monomer | Monomer | (Covalent | Template | | Free |
| MIP | NIPAm | (4-VP) | (MAA) | Crosslink) | (RM:TM) | Solvent | Radical |
| CJG | 68% | 17% | 10% | 5% | Fluorescein | 1,4- | AIBN |
| 292 A | | | | | 4 to 1 | Dioxane | 2% |
| | | | | | | 100 mL | (w/w) |

TABLE 3S
Distribution ratios from dialysis cells in ice water, room temperature,
and above the LCST.

| | | Fluorescein | |
|---|---|---|---|
| MIP (10 g/L) | Temperature | Concentration (μM) | Distribution Ratio |
| CJG 292 A | 0° C. | 1.000 | 1.22 ± 0.038 |
| CJG 292 A | 22° C. | 1.000 | 1.79 ± 0.038 |
| CJG 292 A | 70° C. | 1.000 | 1.53 ± 0.038 |
| CJG 296 A | 0° C. | 1.000 | 1.68 ± 0.038 |
| CJG 296 A | 22° C. | 1.000 | 1.76 ± 0.038 |
| CJG 296 A | 70° C. | 1.000 | 3.67 ± 0.038 |

Listed in Table 3S are the distribution ratios of the two different MIP formulations. Surprisingly, less acid-base non-covalent crosslinks enabled scientifically more selective binding of the MIP for fluorescein. Typically, MIP formulations that use more covalent crosslinks enable better and more selective binding of the template molecule. However, here there can only be so many covalent crosslinks added to the MIP network before it stops acting as a hydrogen and forms a solid material below the LCST. When more acid-base interactions were added to the MIP network they were held to a more general vicinity on the polymer chain, allowing different monomer units to act as a recognition monomer that were previously acting as a base monomer for the non-covalent crosslinking. This lowered the selectivity and overall binding capabilities. Therefore, the less acid-base interactions with the same amount of covalent crosslinks allowed the MIP to keep the acid-base crosslinks more localized and held the original binding site together, thus creating a more selective and high binding recognition center.

### Summary and Overview of Results of Example 2

The goal of the nitrophenol experiments described in Example 2 was to optimize a MIP network that would selectively sense a templated molecule. This was accomplished by exploring various types of polymerization solvents, monomer concentration, amounts of covalent crosslinking, and the numerous non-covalent interactions that could have been used as a crosslink. The results of the studies permitted successful templating and selective sensing of 4-nitrophenol with an MIP. Through the various equilibrium dialysis experiments, distribution coefficients were produced to show how much template was being bound. Use of a non-polar (non-competing) solvent was shown to produce a high binding affinity MIP. The more polar protic solvents offered a higher degree of dissociation of the non-covalent interactions in the pre-polymerized solution. Aprotic polar solvents were found to still disrupt the formation of the hydrogen bonds by accepting the hydrogen bond of the template. In addition, the studies indicated that use of a lower monomer concentration allowed more accurate templating with less chain to chain interactions. In these circumstances the template was found to be able to interact with the recognition monomer more freely and less entanglement was shown through this process.

Results from the studies showed that a combination of non-covalent (acid-base) crosslinks and low levels (for example, but not limited to: 4%) of covalent crosslinks yielded a selective aqueous MIP. It was determined that as with proteins, a combination of crosslinks permitted formation of high affinity binding sites.

### Example 3

### Rapid High Binding Affinity Aqueous Molecular Imprinted Polymer by Non-Covalent Crosslinks: Kinetics and Further Binding Experiments

Studies were performed using the polymer formulation and polymerization parameters determined in Example 2, in conjunction with a fluorescein template to determine the binding capabilities, capacity, and kinetics of prepared MIPs. These studies permitted measurement of the template in real time and at ultra-low concentrations (nM).

### Introduction

Throughout the nitrophenol studies described in Example 2, the preparation and makeup of MIP was optimized. The polymerization solvent, monomer concentration, recognition monomer to template ratio, and crosslinking network were all adjusted in studies performed in Example 2. Using a non-competing, non-polar, solvent with a low concentration of monomer, for example, 0.25 mmol/mL, the polymerization solution was shown to solubilize all monomers, imprint the template molecule with higher recognition, and yielded intra-chain crosslinking. Forming a crosslinking network that consisted of low levels of covalent and non-covalent bonds was shown to produce a MIP that could be handled as a liquid below the LCST and was capable of binding with high affinity and selectivity.

Fluorescein was used as the template molecule in the studies in Example 3 to determine specific binding kinetics, binding affinity, and other parameters of the MIP. Fluorescein is a well-known fluorophore that has been commonly used in microscopy and is soluble in the polymerization solvent described for use in MIP preparation methods herein, and in water (testing solution). Fluorescein has an excitation wavelength at 494 nm and emission wavelength at 512 nm. The use of fluorescein as a template molecule allowed measurement of results at lower concentrations; using a fluorescence spectrometer it was possible to measure the presence of fluorescein at 5 nM. Using fluorescein as the template molecule permitted measurement of binding parameters at lower polymer and template concentrations. The studies included investigation of functionality of prepared MIPs.

### MIP and NIP Polymerization Methods

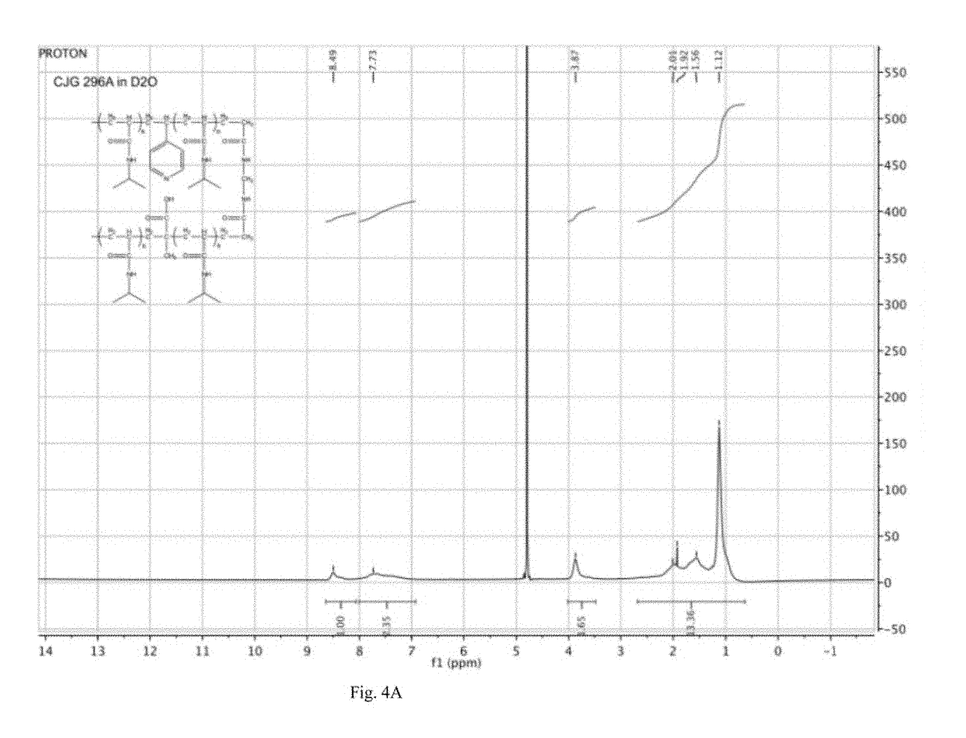

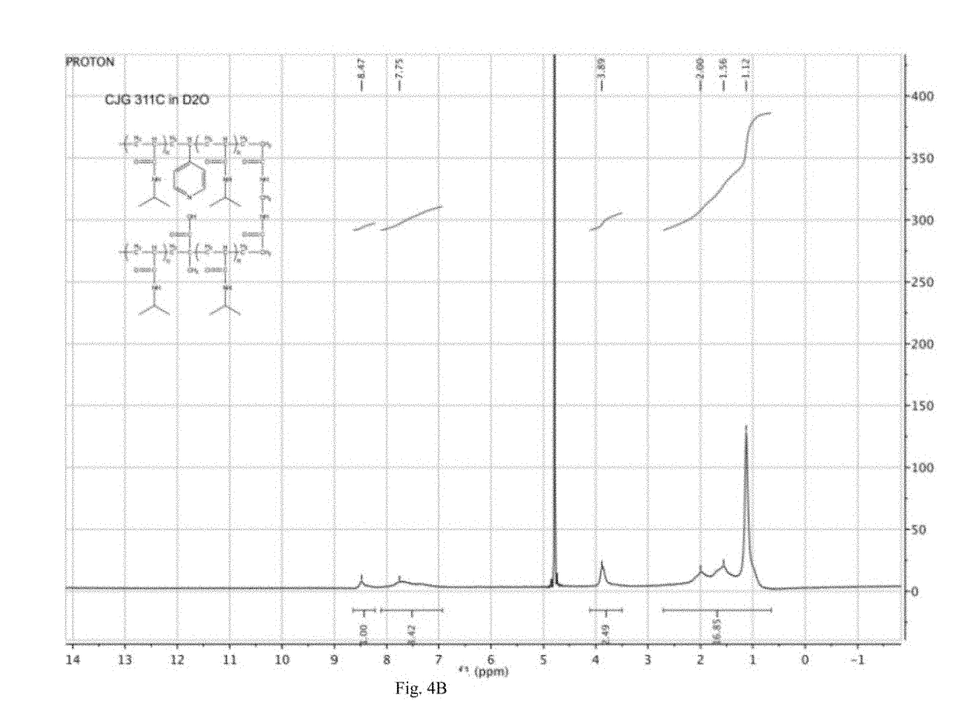

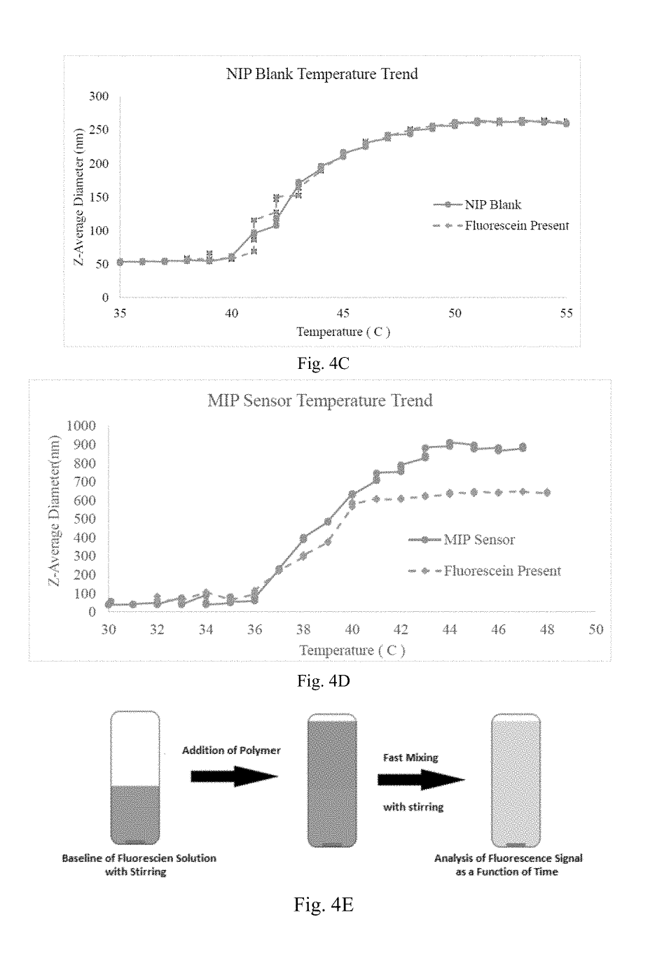

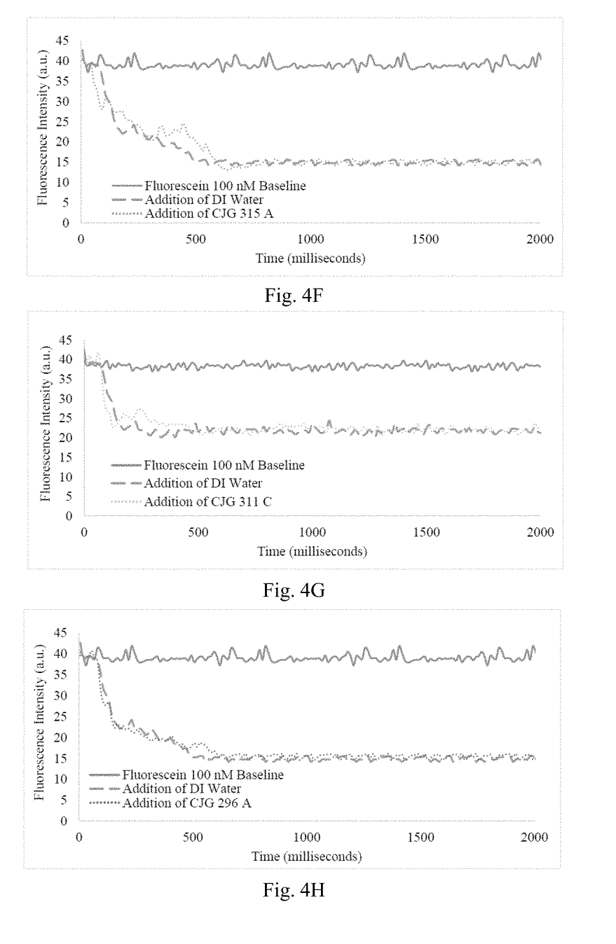

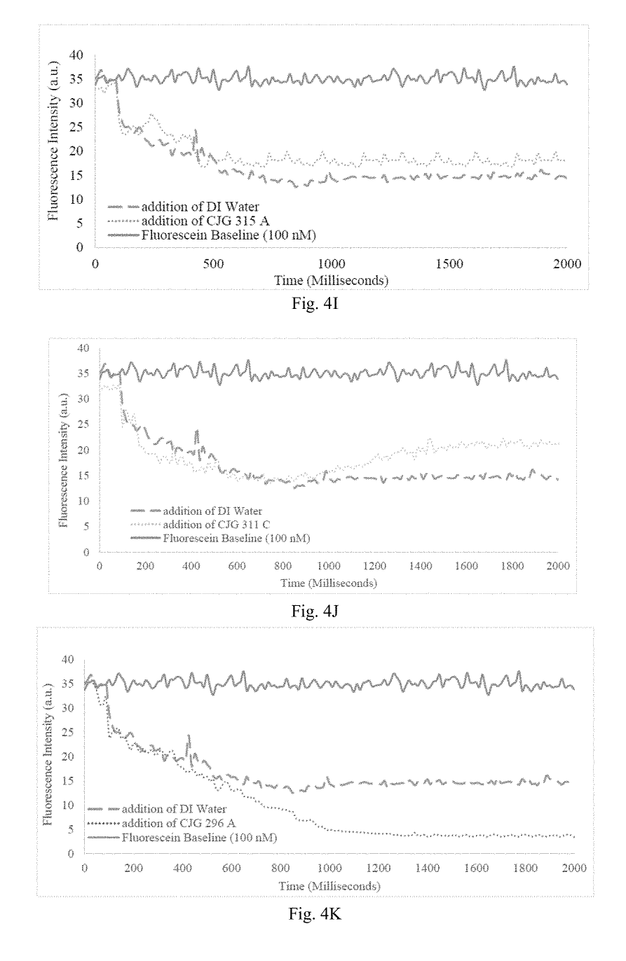

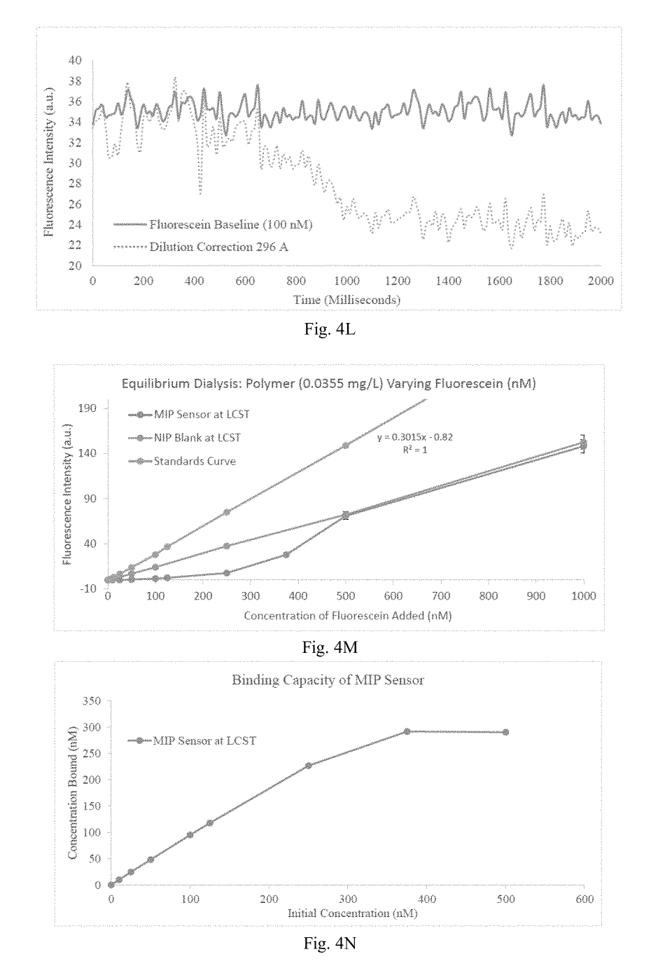

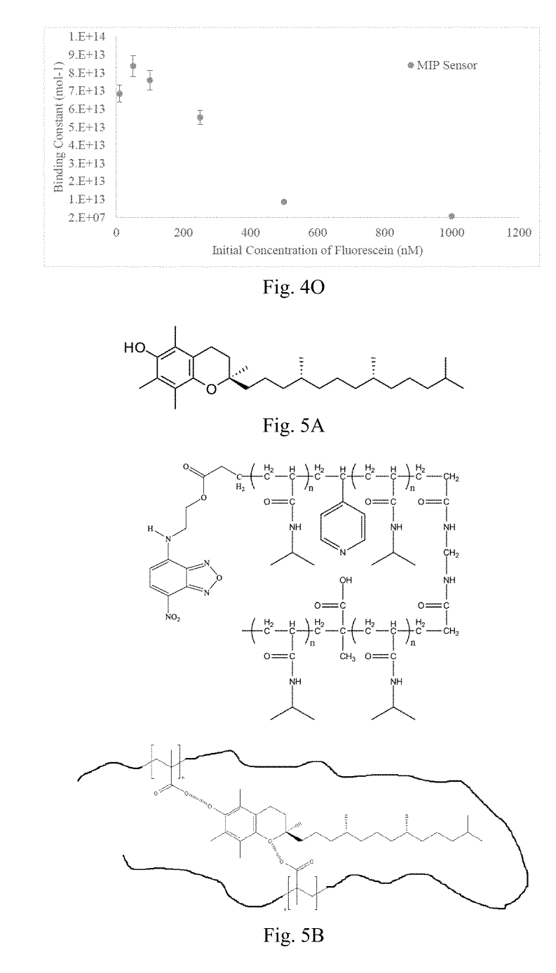

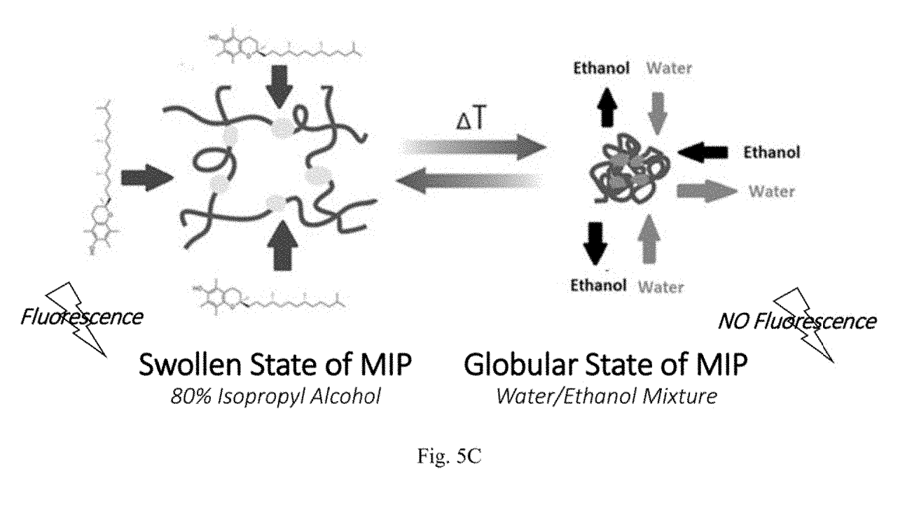

To demonstrate the utility and advantages of this aqueous MIP based chemical sensor, both imprinted (CJG 296 A) and non-imprinted polymers (CJG 311 C) were synthesized and tested for their speed, efficiency, and the optimal conditions at which binding of the targeted molecule occurs. These two polymers were synthesized using the various feed ratios, see FIG. 4A. All polymers consisted of the NIPAm backbone and 4-VP as the recognition monomer and both covalent and non-covalent crosslinks. CJG 296 A was imprinted with fluorescein. CJG 311 C was synthesized in the same manner as CJG 296 A but in the absence of the template molecule.

The feed mixture included the above monomer amounts and was polymerized by reversible addition-fragmentation chain-transfer (RAFT) in 1,4-dioxane (Monomer: RAFT: Initiator, 1000:1:1, AIBN at 70° C.) in the presence of the respective template [Shi, P. et al., (2014) Macromolecules, 47 (21), 7442-7452]. After template removal dialysis, the polymer solution was freeze-dried down to an off-white powder. Known polymer solutions were made in deionized water and given time to solvate/untangle the polymer chains. For the rest of Example 3, CJG 296 A is referred to as “MIP sensor” and CJG 311 is referred to as “NIP”, which stands for: “non-imprinted polymer”, which served as a blank/control.

TABLE 4A
Parameters used for preparing CJG 296A (MIP Sensor) and CJG 311C (NIP blank)

| | | MAA | 4-VP | MBAm | | | |
|---|---|---|---|---|---|---|---|
| | | (Acid | (Base & Rec. | (Covalent | Template | RAFT | |
| Polymer | NIPAm | Monomer) | Monomer) | Crosslinks) | (RM:TM) | Agent | Initiator |
| CJG 296A | 84% | 5% | 9% | 2% | Fluorescein | DDMAT | AIBN |
| (MIP | | | | | (4:1) | (1000:1:1) | |
| Sensor) | | | | | | | |
| CJG 311C | 84% | 5% | 9% | 2% | None | DDMAT | AIBN |
| (NIP | | | | | | (1000:1:1) | |
| Blank) | | | | | | | |

As shown in Table 4A, monomer feed ratios totaled 50 mmol and polymerized by RAFT using 2-(Dodecylthiocarbonothioylthio)-2-methylpropanoic acid (DDMAT) in 1,4-Dioxane. Ratio molar amounts of recognition monomer to template molecule, 4-vinylpryidine: fluorescein, were used as listed above to form templated sites. Degassing was done by freeze-pump-thaw technique and back filling with nitrogen for 5 minutes. Polymerization was heated to 70° C. for seven days with stirring.

### Removal Process for Fluorescein Imprinted Polymer

Using fluorescein as the template molecule not only allowed for binding to be detected at nanomolar levels, but also the removal of the template molecule from the binding sites. Other template molecules in this study could only be detected at the range of a 100 to 10 nanomolar. Being certain that 99.99% of the added fluorescein template was removed was useful for assessments in later binding and kinetics experiments.

Many different combinations of methanol, water, acetone, and 1,4-dioxane with acetic acid or hydrochloric acid were evaluated for removing fluorescein from the templated polymer. It was later discovered that a rotation of removal solvents worked well and lowered the amount of time needed to completely remove fluorescein, from 6 months to 3 months. The first solution was comprised of 50% acetone, 10% 1,4-dioxane, 20% water, 10% acetic acid, and 10% hydrochloric acid. The dialysis bag containing the templated polymer was exposed for a maximum of 12 hours or until the outer solution was dark yellow with fluorescein. Then the dialysis bag was rinsed with deionized water, left in deionized water over night, rinsed again, and then placed in a 0.1 M sodium hydroxide solution for at least two hours. Before the second solution, the dialysis bag was rinsed thoroughly with deionized water to remove all sodium hydroxide. After 3 or 4 rotations of the above methods, a 2-3% hydrogen peroxide solution was added after the final rinse. This was used to oxidize the fluorescein molecule. Then the whole process was repeated with this hydrogen peroxide solution until the interior MIP solution was free of fluorescein. Using an ultra-violet hand lamp the inner solution was observed to determine if fluorescein was still present. These results were then confirmed through fluorescence spectroscopy showing no peak at the fluorescein emission wavelength (Ex λ=494 nm and Em λ=512 nm).

Long template removal times were determined not to be totally an effect of improper removal solvent, however, a MIP conformation problem. It was hypothesized that the binding sites were being blocked through the aggregation of the chain and neighboring chains. After synthesis these MIPs are drastically entangled. Alternating the removal solutions helped induce the different conformations that unblock some of the binding sites. In addition to the removal solvent, the temperature at which it is present also affected template removal. The MIP should open up and be soluble below the LCST. Although, even when the temperature is reduced the template was so strongly bound that it stayed on the polymer even well below the LCST.

### Characterization of MIP Sensor and NIP Blank

With removal of the fluorescein template molecule confirmed by fluorescein's spectroscopy the MIP sensor and NIP blank were lyophilized down, which resulted in an off-white powder. This allowed use of various characterization techniques (GPC, NMR, and DLS) to confirm the structure [Sellergren, B. et al., (1988) Journal of the American Chemical Society, 110 (17), 5853-5860], determine a molecular weight, a polydispersity index [Hong, C. Y. et al., (2004) Journal of Polymer Science Part A: Polymer Chemistry, 42 (19), 4873-4881], and the thermal phase transition of the polymers in various solutions [Odian, G., (2004) Principles of Polymerization 4th ed.; Wiley-Interscience: New York; Tang, L. et al., (2009) Chemical Communications, (33), 4974-4976]. This was necessary for assessing current and future results of these sensors.

Gel permeation chromatography was used to determine the molecular weight and polydispersity index of the two polymers Table, 4B [Wu, Q. et al., (2014) J. Membrane Sci., 471, 56-64; Savariar, E. N. & Thayumanavan, S., (2004) J. Polymer Sci. Part A: Polymer Chemistry, 42 (24), 6340-6345]. These results confirmed that an appropriate MWCO dialysis tubing was being used (10-12,000 MWCO) during the removal process. In addition, knowing the molecular weight enabled a correct calculation of monomer present in both the NIP and MIP. Using the NMR listed below, and these results, a molar amount of 4-VP present could be calculated. Assuming that percentage of MAA present would also interact with a similar percentage of the 4-VP. The remaining 4-VP present would make up the binding sites, corresponding to the ratio 4-VP (recognition portion) to the template molecule (fluorescein). After this calculation the amount of binding sites was known [Dan, M. et al., (2013) Journal of Polymer Science Part A: Polymer Chemistry, 51 (7), 1573-1584].

TABLE 4B
Gel permeation chromatography results for the MIP sensor and NIP blank.

| Sample ID | Mn | PDI |
|---|---|---|
| CJG 296 A (MIP Sensor) | 13,800 | 2.18 |
| CJG 311 C (NIP Blank) | 13,600 | 2.15 |

For the gel permeation chromatography (some results shown in Table 4B), the samples were dissolved in 3 g/mL in eluent (water with 0.2 M sodium nitrate and 2 weight % sodium azide). The supernatant (0.5-1 mL) was filtered through a PTFE syringe filter (0.45 μm pore size) into a clear vial. With 10-20 μL methanol was added to each sample then loaded into the GPC.

Polymers synthesized and described in the Examples, are listed as “feed ratios”. Feed ratios are not the exact ratio of monomers present in the polymer network. Determining the exact composition of polymer (percentage of each monomer present) was not necessary for certain experiments, but once the polymer formulation and method was fully optimized, it was helpful to know the number of binding sites present in the MIP. Knowing this value increased the accuracy of additional testing with regards to what was actually present in the tested materials. Therefore, NMR spectroscopy was performed for the MIP sensor (CJG 296 A) and NIP blank (CJG 311 C) in D₂O, which confirmed that all monomers present in the feed ratio had shifts within the NMR spectra, confirming that at least some of the monomers had polymerized within the polymer. FIGS. 4A and 4B show NRM results from MIP sensor and MIP blank, respectively.

In addition, by integrating the peak heights it was possible to obtain a percentage range of the 4-vinylpryidine present, ˜20% of the total polymer. This is more than what was planned by the feed ratio. This difference was due to the differences in reactivity ratios of monomers during a living polymerization. Based on the NMR data, it is expected for more binding sites to be formed within each polymer chain and less NIPAm would be present.

### MIP Sensor Aggregate Size Versus Temperature

The MIP sensor was optimized as described in Example 2. Because the polymer was primarily NIPAm suggested that would behave similarly to the functionality of that homopolymer. Using some 4-vinylpyridine and methacrylic acid would adjust the LCST value slightly [Kim, K. S. & Vincent, B., (2005) Polymer Journal, 37 (8), 565-570]. The amount of covalent crosslinking, MBAm, being used still resulted in the MIP being a liquid at low temperatures and would result in a wider temperature range to eventually go from globule to polymer aggregates. With this newly optimized MIP sensor, further characterization and binding abilities were explored. The polymers LCST was determined through DLS experiments by measuring particle size versus temperature. The same was done with the NIP blank. This can be seen in FIGS. 4C and 4D. The solid line trace is the polymer sample present in 1000 μL of water at a 0.1 g/L concentration and after being filtered twice with a poly (styrene) 0.45 μm syringe filter. The filtering ensured that no dust or other particulates were present in the solution before the experiment. This temperature increased from 30° C. to 50° C. for the MIP sensor and 35° C. to 55° C. for the NIP blank. This allowed the LCST values for the two different polymers to be compared. A new sample of polymer was used for the same procedure but in the presence of fluorescein, 100 μL of 1 μM, filtered and added to the polymer sample. These results are represented by dashed lines the in FIGS. 4C and 4D.

From FIGS. 4C and 4D it can be noted that the LCST value for MIP sensor is between 38° C. and 42° C. and the NIP blank is between 42° C. and 46° C. This is represented within the solid line traces. It is also worth noting that the MIP sensor swells and aggregates to a larger size, ˜900 nm, and the NIP blank is ˜250 nm. This is a threefold size change from the NIP blank to the MIP sensor. Size distribution between with the imprinting technique allows for uniform and larger polymer networks. The imprinting technique allows the 4-vinylpryidine to interact with the template molecule, here fluorescein, before polymerization. This allows a conformational change, or other type of change, which allowed the monomer to become more reactive in the polymerization process. This is noted by Table 4B, the GPC results of the MIP sensor and NIP blank. Because the molecular weights and PDI were roughly the same, the addition of more or less 4-vinylpyridine was identified as a reason for this drastic aggregate size difference.

When the same procedure was repeated in the presence of fluorescein, the NIP blank had the same polymer aggregate size results as when it is just in water. However, the MIP sensor showed a decrease in polymer aggregate size at ˜600 nm and leveled off at a lower temperature, 40° C., instead of when there was no fluorescein present, ˜900 nm and 43° C. These curves are highly reproducible and because of this the trends display a unique phenomenon [Yadav, S. et al., (2011) Analytical Biochem., 411 (2), 292-296]. The decrease in aggregate polymer size when at lower temperatures is evidence that the MIP sensor was binding with the templated molecule, fluorescein, allowing the MIP sensor to not aggregate further after binding is completed around the fluorescein molecule. This size change was noted to happen at the LCST. If the MIP sensor yields a size change difference with and without the templated molecule present, then it was expected that binding would have the strongest affinity at the LCST. When the polymer is transitioning from a random coil to an aggregate particle it is in the globular transition state. This state would allow the MIP sensor to stabilize its conformation. There would be maximum flexibility of the MIP when it is held at the LCST because hydrophobic collapse is just beginning to occur. Conformational changes between random coil and globule would be expected around the LCST. Allowing the polymer's binding sites to have the higher hydrogen bonding affinity because the hydrophobic crosslinking of the MIP would be favored. At higher temperatures the hydrogen bonding about the amide group is less favored and eventually allows the NIPAm based polymers to collapse and aggregate. In addition, each unit of the acid-base crosslinking network is being held in the vicinity of each other by the covalent crosslinks of MBAm. With the polymer chain in the globular state, they can be brought together and hold the binding site in the same size and shape as it was formed. This form would allow for higher binding affinity and better selectivity.

### Temperature and Polymer Concentration Binding Affinity

Equilibrium dialysis experiments were completed at various temperatures to determine how temperature affects binding affinity. The selected temperatures (23° C., 40° C., and 80° C.) were chosen to be below, at, and above the LCST so that the MIP sensor is in the random coil, globular, and aggregate state. The resulting distribution ratios for the MIP sensor and NIP blank are listed in Table 4C.

TABLE 4C
Results of binding with various temperatures.

| | Polymer | Fluorescein | |
|---|---|---|---|
| | Concentration | Concentration | Distribution |
| Polymer | (mg/L) | (nM) | Ratio |
| MIP Sensor @ 23° C. | 1 | 1 | 2.748 |
| MIP Sensor @ 40° C. | 1 | 1 | 4.567 |
| MIP Sensor @ 80° C. | 1 | 1 | 4.063 |
| NIP Blank @ 23° C. | 1 | 1 | 1.041 |
| NIP Blank @ 40° C. | 1 | 1 | 1.321 |
| NIP Blank @ 80° C. | 1 | 1 | 1.257 |

Each dialysis block was held at the listed temperature for seven days to ensure that proper equilibration was achieved. The results show that below the LCST when the MIP sensor is a random coil, it has a distribution ratio of 2.75. This shows that there is some binding; however, when the MIP sensor is held at the LCST the amount of fluorescein bound is almost doubled. This data indicated that binding increases once the MIP sensor solution is brought to the LCST. Furthermore, when the MIP sensor was kept above the LCST, it still bound better than when it was below the LCST, but still was slightly worse in its binding affinity, with a distribution ratio of 4.06. This provided additional evidence that the greatest binding affinity for the template is at the LCST. When the MIP sensor is above the LCST, the polymer chains aggregate too quickly, not allowing the binding site to be open long enough before they aggregate and collapse shut.

The idea of the polymer chains collapsing and blocking the template molecule from effectively binding to the MIP was confirmed by measuring binding affinity versus the MIP concentration using equilibrium dialysis Table 4D. At very high temperatures the MIP is in complete hydrophobic collapse and this blocks some of the binding sites. This was also observed during the removal experiments in this Example.

Previously, it was believed that more MIP present in solution would bind more template because more binding sites are present. This would produce higher distribution ratios. This was not observed, as shown in Table 4D. Instead distribution ratios increased greatly as the concentration decreased.

TABLE 4D
Resulting equilibrium dialysis experiments with various
polymer concentrations.

| | | | | Distribution Ratio |
|---|---|---|---|---|
| Polymer | Polymer | | Fluorescein (nM) | at the LCST |
| MIP Sensor | 10 | (g/L) | 100 | 3.668 |
| MIP Sensor | 1 | (g/L) | 100 | 4.091 |
| MIP Sensor | 0.1 | (g/L) | 100 | 4.573 |
| MIP Sensor | 0.0355 | (mg/L) | 100 | 9.891 |
| NIP Blank | 10 | (g/L) | 100 | 1.726 |
| NIP Blank | 1 | (g/L) | 100 | 1.343 |
| NIP Blank | 0.1 | (g/L) | 100 | 1.279 |
| NIP Blank | 0.0355 | (mg/L) | 100 | 1.098 |

Subsequently, as the concentration of the MIP sensor and the NIP blank went down, binding affinity greatly improved, in respect to the imprinting technique. There is theoretically 4.5 mmol (9%) of the 4-VP polymerized within the MIP. Five percent of that is dedicated to the acid/base crosslinking, which leaves 2 mmol (4%) for the formation of binding sites. If it takes 4 monomer units of 4-VP to create one binding site, then there should be 0.5 mmol of binding sites present (1%). The NMR data suggested that there was ˜20% 4-VP present, suggesting that there could be up to ˜2 mmol of binding sites within the MIP polymer sample. Either theoretical or rough calculations suggested that there were still a huge number of binding sites unexposed.

With less polymer present, the binding sites were more exposed to the presence of various molecules. The imprinting technique is selective enough to only bind with the molecule present. The MIP sensor represents the highest binding affinity at low concentrations and when it is present at the LCST. Having less polymer present in the globule state allows the binding sites to be strongly formed and unblocked from neighboring polymer chains. This creates a high binding affinity and binding constant for this MIP sensor.

### Kinetics Studies

The MIP sensor was shown to bind fluorescein with high affinity; but this was shown using equilibrium dialysis, which took upwards of seven days to complete. One purpose of an aqueous non-covalent MIP is to bind quickly and selectively. The MIP binding kinetics were investigated through a series of experiments using fluorescence spectroscopy [Sridharan, R. et al., (2014) Biochim. et Biophys. Acta, 1838 (100), 15-33].

A concentration of fluorescein that is within the theoretical binding capacity of MIP sensor was prepared to test how rapid the MIP sensor would bind. Using a fluorescence spectrophotometer and its kinetics function with a stirred 4 mL quartz cuvette, the kinetics experiment was carried out with an excitation and emission wavelength of 492 nm and 512 nm respectively. 2.000 mL of 100 nM fluorescein was added to the cuvette and baseline fluorescence. Then a similar aliquot of polymer solution was added to the cuvette and measured over time. This was done with a variety of polymer solutions and deionized water aliquot to test for the dilution factor. This experiment is illustrated in FIG. 4E.

FIGS. 4F-4H depict the binding kinetics of the following three polymers: poly (NIPAm), NIP blank, and MIP sensor. All were measured at room temperature for 30 minutes (only 2 seconds displayed in figures). The addition of water to the fluorescein solution shows how diluting the solution affects the fluorescent signal. If the fluorescent signal was below the water addition signal, binding of fluorescein would be assumed.

Even though these kinetic experiments were done over 30 minutes, only two seconds were displayed in these figures to compare to later results. Poly (NIPAm) and the MIP sensor followed the trend of the addition of deionized water, which is indicative that the polymer solution was not binding with the fluorescein template and the fluorescence signal was dropping due to a dilution affect. The NIP blank had a slightly higher fluorescence signal than just the addition of deionized water in the first few seconds. Then the fluorescence signal actually increased and then leveled off at the fluorescence intensity attributed to dilution of the fluorescein solution. The signal increasing and decreasing eventually was attributed to the mixing of the polymer solution in the cuvette.

These experimental results were expected because they are done at room temperature. Binding was exhibited at room temperature during the previously described equilibrium dialysis experiments (see Example 2); however they were allowed to equilibrate for days. Additionally, binding below the LCST might be so minimal that a signal intensity change was not seen. This experiment measured the immediate binding of the MIP sensor and other polymer samples. Using the results from this kinetics study and the equilibrium dialysis results it was concluded that the MIP sensor binds with the templated fluorescein molecule slowly at room temperature, or not enough binding occurs to get a fluorescence intensity change. Based on these results, the same experiments were performed at the polymers' LCST.

Using the same procedure, the cuvette holder was set to each of the polymers' individual LCSTs, poly (NIPAm) 32° C., NIP blank 44° C., and MIP sensor 40° C., and the fluorescein solution was allowed two minutes to equilibrate to the temperature. Each polymer solutions' aliquot was placed in a water bath with the appropriate temperature for about 10 minutes. Results are shown in FIGS. 4I-4L.

Poly (NIPAm) affected the fluorescence signal to drop, not exactly the same but similarly to the deionized water dilution affected signal. The leveling off of the signal was observed for the entire 30 minutes. The NIP blank was observed to decrease the fluorescence signal but then radically increase before leveling off where the poly (NIPAm) solution did. This phenomenon could be explained by mixing of the polymer solution.

Shown in FIGS. 4K and 4L is the binding kinetics of the MIP sensor when at its LCST. The signal rapidly decreased, within milliseconds, and eventually leveled off below 5 a.u. This indicated that the MIP sensor had bound with the fluorescein template and bound almost all of template that was possible. According to equilibrium dialysis results, the amount of fluorescein bound, ˜95% of the total concentration was expected. Having done this in a matter of seconds indicated that this MIP sensor has a rapid and strong affinity to the templated molecule. With these rapid binding results from the MIP sensor, the exact amount of fluorescein that is capable of being bound was next examined.

### Binding Constant and Binding Capacity

To determine the binding constant and binding capacity of a prepared MIP sensor or the amount template the polymer is capable of binding, equilibrium dialysis was utilized. Allowing the MIP sensor concentration to stay constant, 0.035 mg/L, the concentration of fluorescein present in each block was varied from 0 nM to 500 nM. In FIG. 4M, fluorescence intensity was plotted versus the initial concentration of fluorescein being present in the equilibrium dialysis cell. This was completed for the MIP sensor and the NIP blank, the fluorescein intensity of the initial concentration solutions was also plotted. This graph depicts the non-binding of the NIP blank. With equilibrium dialysis, the fluorescein concentration after equilibration time will always be half the original amount. Therefore, when evaluating the graph to see if binding is occurring with the MIP sensor, a value below the linear line of the NIP blank depicts such.

The binding capacity was concluded and displayed in FIG. 4N. This is the amount of the MIP that can completely bind. This was confirmed because the NIP blank showed no fluorescein to be trapped within the polymer and no amount of fluorescein bound with the NIP blank. The amount of fluorescein being introduced to MIP sensor is being bound linearly until the concentration of fluorescein reaches 250 nM. This is where binding of fluorescein begins to decrease. As the concentration of fluorescein is increased to 375 nM the plotted line levels out. It is at this point that the binding capacity of this concentration of MIP sensor has been reached, according to the equilibrium dialysis parameters. It is also worth noting that the MIP sensor can sense a very small presence of templated molecule, at a parts-per-billion scale.

In FIG. 4O, the binding constants of each MIP that was placed in the presence of the various fluorescein concentrations are graphed versus the initial concentrations, example calculation shown below. FIG. 4O shows that the binding constant eventually decreases with the addition of more fluorescein, depicting that the MIP has reached its binding capacity. However, because this is based off the amount present on the polymer side and not the amount bound, additional experiments were performed (see Example 5) that allowed removal of the MIP from the fluorescein solution.

Calculation of Binding Constant: the following is an example calculation for MIP equilibrium dialysis results with 50 nM fluorescein spin down experiment accounting for equilibration of fluorescein within the dialysis cell.

$$25 nM Standard Fluorescein Intensity ( a . u . ) - M I P with 50 nM Fluorescein After Equilibrium Dialysis Inentsity ( a . u . )$$

$$6.613 - 0.492 = 6.121$$

$$\frac{Difference in Fluorescence Intensity}{Slope of the Calibration Curve} + Y intercept = M I P Bound Fluorescein = \frac{6.1212}{0.3015} + 0.820 = 21.122 nM = 2.112 \times {10}^{- 8 } M Bound Fluorescein$$

$$Fluorescein Present = 5.00 \times {10}^{- 8} M$$

$$\begin{matrix}M I P Present = 1.40 \times {10}^{- 4} L \times 3.55 \times {10}^{- 5} \frac{grams}{L} \\ = \frac{4.97 \times {10}^{- 8} grams}{13.800 \frac{grams}{mole}} \\ = \frac{3.60 \times {10}^{- 12} moles}{0.0014 L} \\ = 5.04 \times {10}^{- 15} M\end{matrix}$$

$${K}_{Binding} = \frac{M I P Bound Fluorescein}{Fluorecein \times M I P}$$

$${K}_{Binding} = \frac{2.112 \times {10}^{- 8} M}{5.00 \times {10}^{- 8} M \times 5.04 \times {10}^{- 15} M} = 6.84 \times {10}^{13}$$

### Summary and Overview of Results of Example 3

Results of these experiments demonstrated the preparation and testing of a fluorescein templated MIP with a poly (NIPAm) that holds the formed binding site with predominantly non-covalent acid-base crosslinks. Sensing of the template was at the LCST. Binding of the template was identified as occurring with a high and rapid affinity. When compared to conventional MIPs and other forms of chemical sensing, the binding of the template occurred much more quickly. The binding was demonstrated through binding kinetic experiments, and was shown to occur within seconds with the prepared MIP. This MIP sensor was observed to be a low viscosity hydrogel material. These reasons support a conclusion that prepared MIP sensors, as described herein, will be useful in numerous applications for chemical sensing and compound separations.

### Example 4

### Inkjet Paper Based Printed Moleculary Imprinted Copolymers: Development of a Chemical Sensor

Inkjet Printing with Embodiment of Prepared Aqueous MIP with Fluorescence Response

One of the goals of experiments described herein was to use this MIP as a separation technique, or as a sensor. Using the designed MIP as a separation technique can be done in solution; however, using the MIP as a sensor might also be done by applying the MIP to a substrate. The prepared non-covalently crosslinked, acid and base crosslinks, MIP (CJG 296 A) has a small diameter (˜100 nm) when below the LCST, a finding confirmed by DLS data presented in Example 3. This particle size is ideal for inkjet printing onto a paper substrate. Nozzles are typically 20-30 μm in diameter [Derby, B., (2010) Ann. Rev. Materials Res., 40, 395-414]. Studies were performed to combine ink jet printing with prepared MIPs to create chemical sensor arrays, using an aqueous polymer solution, which unlike other types of MIPs, solid polymer particles, can be dispersed through a fine nozzle [Park, J.-U. et al., (2007) Nature materials, 6 (10), 782-789]. Modern inkjet printers support the creation of precise and contactless deposition of pico-liter sized droplets of a substrate onto paper or other substrate [Park, J. & Moon, J., (2006) Langmuir, 22 (8), 3506-3513]. This technology can be used in fabrication technology methods because of its high reproducibility and low cost [Abe, K.; et al., (2008) Analytical Chem. 80 (18), 6928-6934; Izdebska, J. & Thomas, S., (2015) Printing on Polymers: Fundamentals and Applications. William Andrew].

The prepared MIP was able to be handled as a liquid below the LCST and had the fluid characteristics of ink [Calvert, P., (2001) Chemistry of Materials, 13 (10), 3299-3305]. These characteristics of the prepared MIP supported use of an ink jet printer to print the prepared polymer solution (MIP) onto a substrate, such as a paper-based substrate [Yamada, K. et al., (2015) Angewandte Chemie International Edition, 54 (18), 5294-5310]. Such printing would result in a substrate capable of having selective binding properties. Experiments are performed to create an aqueous MIP solution that could be used to assess levels of α-tocopherol in bodily fluids. An aqueous MIP ink as a sensor array will be a reproducible and universally stable analytical tool that is capable of performing measurements for an in home setting or in developing countries with minimal resources [Abe, K. et al., (2010) Anal Bioanal Chem 398 (2), 885-893]. A flexible molecularly imprinted polymer was prepared. This was done with low levels of covalent and acid-base crosslinks. This MIP was capable of rapid and selective detection of templated α-tocopherol and is also amenable to inkjet printing. However, printing the MIP solution on to the substrate was just one component to making the chemical sensor. Another component is developing a visible response that is relative to the molecule present. Studies were performed to develop and determine a fluorescent label that would change its emission characteristics upon template binding.

It was proposed that achieving a visible response for this sensor would be accomplished by the addition of a synthetic fluorophore monomer to the MIP network. This fluorescent monomer unit would react to the swelling and shrinking of the MIP, binding specifically with the template. This technology can then be used to develop a chemical sensor with an “on/off” fluorescence response. This would enable the MIP to quench its own fluorescent emitting light and turn off when sensing the template. The MIP would have to be capable of being inkjet printed on to paper substrates and yielding selective reproducible responses to the template molecule when semi-dried.

### Template Molecule Preparation

The selected template molecule: Vitamin E is in a category of ten lipid soluble molecules that can be divided down into two basic forms, tocopherol and tocotrienol. These molecules are used as fat-soluble antioxidants in the body that can terminate the production of reactive oxygen species that can be formed when lipids, in the body, go through oxidation. Vitamin E, specifically the α-tocopherol form, has been shown to protect cells by having the ability to quench free radicals by reducing the oxidative strain [Rigotti, A., (2007) Molecular Aspects of Medicine, 28 (5-6), 423-436]. Monitoring and controlling the levels of such antioxidants is important because unmonitored levels can be harmful for cells. There are a variety of studies that suggest that supplements of vitamin E may lower a patient's risk of chronic diseases, such as Alzheimer's disease, Parkinson's disease, cataracts, ischemic heart disease, and certain types of cancers [Herrera, E. & Barbas, C., (2001) J. Physiol. Biochem., 57 (1), 43-56].

FIG. 5A shows the chemical structure and physical form of α-tocopherol. There are three stereocenters in α-tocopherol, making this a chiral molecule [Jensen, S. K. & Lauridsen, C., (2007) Vitamins & Hormones, Academic Press; Vol. 76, pp 281-308]. In addition, it features a chromane ring with a hydroxyl group that can donate a hydrogen atom, a possible recognition monomer interaction, to reduce free radicals [Burton, G. W. & Ingold, K. U., (1981) Journal of the American Chemical Society, 103 (21), 6472-6477]. This might reduce the polymerization of the MIP if the recognition monomer is not interacting with this hydrogen binding site first. There is also a long hydrophobic side chain that is used to penetrate into biological membranes [Rigotti, A., (2007) Molecular Aspects of Medicine, 28 (5-6), 423-436]. This chain makes it almost insoluble in water and explains why α-tocopherol's physical form is as an oil. Other testing solvents are also examined.

### Experimental Information

Studies were performed to develop a MIP solution ink that can be used as a chemical sensor to sense α-tocopherol, the template molecule. The focus was on developing a paper-based analytical measuring device that could give a visual (fluorescent light) response when the template was present. Fluorescence-based chemical sensing would be convenient as an application because it would not require instrumentation but just a visible signal [James, T. D. et al., (1994) Angewandte Chemie International Edition in English, 33 (21), 2207-2209]. At the start of the studies, it was determined that a fluorescence monomer would be used because it can be implemented with any substrate. In solution, proof of concept measurements were to be analyzed first and adjustments made accordingly. In addition, the final MIP ink had to be adjusted according to the printable ink specifications [Komuro, N. et al., (2013) Anal Bioanal Chem, 405 (17), 5785-5805]. The final goal was to print the MIP ink onto a paper substrate and to determine whether there was a fluorescence decrease selective to (resulting from) the presence of the template molecule.

The MIP sensor that was used is the same optimized RAFT MIP that was developed in Example 2 and further optimized in Example 3, but it was polymerized in the presence of α-tocopherol. FIG. 5B shows an outlined molecular structure of the MIP with the fluorophore monomer unit. For use in the Example 4 studies, the functional monomer was switched from 4-vinylpyridine to methacrylic acid. The interacting monomers of 4-vinylpyridine (base) and methacrylic acid (acid) will form this acid/base crosslinking system. Methacrylic acid was used as the functional monomer that reacted and bound with the template molecule; the binding was formed by hydrogen bonding. Methacrylic acid has two functions within the polymer; therefore, a calculated amount was to be added into the polymer solution so there was enough acid monomer available to interact with the base monomer for the crosslinking system, and still be enough acid monomer to form the calculated amount of binding sites within the polymer.

Alpha-tocopherol is only very slightly soluble in water; therefore, amounts of ethanol had to be used to ensure that the alpha tocopherol sample fully dissolved [Cawley, J. D. & Stern, M. H., (1954) Water-soluble tocopherol derivatives]. Using this solvent system enabled the functionality of the MIP sensor. Shown in FIG. 5C is the theory behind how the polymer network will collapse and turn off fluorescence. Shown in Example 3, the phenomena was noticed when the MIP was in the presence of the template molecule and the collapse of the MIP was less and hindered overall aggregation. This created smaller aggregate particles. When the MIP is solvated with an ethanol and water mixture, the MIP is capable of absorbing and binding with some water molecules, but still in a slightly globular state at room temperature because of the ethanol present. These results agreed with the results from a prior publication by Nakayama, et al., and demonstrated that the template molecule when bound, stopped the MIP from aggregating [Watanabe, M. et al., (1998) J. Am. Chem. Soc. 120 (22), 5577-5578]. Keeping more of a hydrophilic conformation enabled the NBD to be more accessible to water and decreasing the fluorescence signal. There would still be a varying amount of both solvents within the MIP network. The more template being bound, the more the MIP will be held in place and less likely to aggregate. This will enable the fluorescence to be decreased more according to how much is bound.

The design of the chemical sensor was planned and engineered, see FIG. 5D. The black traces are all wax ink that are hydrophobic and hold the aqueous testing solution within the outlined channel. The MIP aqueous ink was printed in the middle of the channel. The lower bulb is where the analyte sample in question would be placed. With the sensor being printed onto paper it can utilize capillary action to travel up the paper sensor within the wax bound channel. The sample is allowed to cross over the printed MIP and is able to react within the channel. The sample solution will flow up the channel to the detection area and recognition area [Yamada, K. et al., (2015) ACS Applied Materials & Interfaces, 7 (44), 24864-24875]. This is where it is allowed to fluoresce or not fluoresce, without or with the presence of the template, respectively.

FIG. 5E provides an outlined graph of fluorescence intensity of the printed MIP, MIP with analyte, and analyte only. When the fluorescence MIP is printed within the wax outlined paper channel it is the highest fluorescence intensity that the MIP should be. When the MIP interacts with the analyte with template present it should be decreased dramatically so that it can be visibly determined with the naked eye. The template analyte will also give some fluorescence intensity signal, depicted by the lowest trace.

### Method and Development of a Paper-Based Sensor

Optimizing the MIP formulation was done to ensure that the MIP is selectively binding the template with high affinity. Addition of more recognition monomer was added within the MIP to ensure binding of α-tocopherol. The long aliphatic chain is hydrophobic and less stable to hydrogen bond with the recognition monomer [Sherrington, D. C. & Taskinen, K. A., (2001) Chemical Society Reviews, 30 (2), 83-93]. Therefore, increasing the amount of recognition monomer will allow the hydrogen donating portion of MAA to more accurately surround and fully saturate the template's hydrogen binding sites. The hydrogen accepting portion of MAA will increase acidity and help hold the aliphatic chain in place to allow templating to occur.

The MAA feed ratio was increased to 15% of the mole concentration, instead of 4% that was previously stated in Example 3, for the fluorescein template. The same amount of acid/base non-covalent crosslinking and covalent crosslinking was used. This portion of the MIP formula was not affected by the template molecule change and as a result the binding affinity was strong. Below in Table 5A is a polymer formulation for the final optimized MIP sensor and NIP blank. After the proof of concept experiments the fluorophore monomer was added. For every 50 mmol of monomer present, 200 mg of NBD-AE was used. This was the MIP used throughout these experiments and is represented in the following results.

TABLE 5A
Polymer formulations for the MIP sensor and NIP blank polymers.

| Polymer | NIPAm | MAA | 4-VP | MBAm | RAFT Agent | Solvent | Template |
|---|---|---|---|---|---|---|---|
| MIP | 71% | 20% | 5% | 4% | DDMAT | 1,4- | α- |
| Sensor | | | | | (1000:1:1) | Dioxane | tocopherol |
| NIP | 71% | 20% | 5% | 4% | DDMAT | 1,4- | Absence |
| Blank | | | | | (1000:1:1) | Dioxane | |

### Binding Affinity and Selectivity

New HDPE equilibrium dialysis cells were designed and manufactured by the UNH engineering department for use with organic solvents. The testing solvent that the MIP sensor and template was solvated in was a 50/50 mixture of 95% ethanol and MilliQ deionized water. The MIP was still capable of swelling and shrinking in solution and α-tocopherol dissolved in this solvent. In addition, this would be the solution that would be present during sampling on the paper substrate. Before equilibrium dialysis testing could be completed, calibration of these new cells was done. Template solutions were placed on the one side of the dialysis cell and the ethanol/water mixture was placed on the other. Various blocks were allowed to equilibrate at different times and temperatures to ensure that the testing dialysis cells were given enough time to properly equilibrate. Table 5B provides representatives of the various equilibration times.

TABLE 5B
Calibration of the HDPE equilibrium dialysis cells

| Equilibration Test | Distribution Ratio |
|---|---|
| After 12 Hours ~23° C. | 3.496 |
| After 12 Hours 0° C. | 9.470 |
| After 12 Hours 70° C. | 1.027 |
| After 24 Hours ~23° C. | 2.416 |
| After 24 Hours 0° C. | 4.664 |
| After 24 Hours 70° C. | 1.001 |
| After 48 Hours ~23° C. | 1.353 |
| After 48 Hours 0° C. | 1.363 |
| After 53 Hours ~23° C. | 2.848 |
| After 72 Hours ~23° C. | 1.090 |
| After 53 Hours 0° C. | 9.997 |
| After 72 Hours 0° C. | 2.897 |
| After 96 Hours 0° C. | 0.968 |

After 96 hours all equilibrium dialysis cells where equilibrated. Therefore, all dialysis blocks were allowed 96 hours to properly equilibrate and enable the MIP to bind the template. The MIP sensor and the NIP blanks then could be efficiently tested. The following equilibrium dialysis cell conditions were used: MIP, NIP, poly (NIPAm) samples were at a concentration of 10 g/L with 33 mg/L of either α-tocopherol or 4-hydroxycoumarin (4-HC). To test selectivity 4HC was used because it has a similar base structure of α-tocopherol. The NIP was used to determine if the imprinting method is working selectivity and with high affinity. Poly (NIPAm) was used to determine if the presence of the functional monomer and crosslinking network present a viable recognition site. FIG. 5G provides distribution ratios of the various equilibrium dialysis experiments done at room temperature displayed in a bar graph.

The MIP sensor has high affinity for the template molecule, and although the MIP sensor still binds with 4-HC significantly, it binds with the template 5 times as much. The NIP sample revealed that this polymer formulation will arbitrarily bind with these types of compounds; however, the imprinted technique is still binding the α-tocopherol at a higher rate. Poly (NIPAm) gave the predicted result of just equilibrating within the dialysis cell. This MIP sensor showed great promise with the equilibrium dialysis testing for binding affinity and relative selectivity.

### Synthesis of Fluorophore Monomer

A fluorophore monomer was designed and synthesized such that it emitted when the MIP sensed the template molecule in an ethanol and water mixture. A nitrobenoxadiazole (NBD) amine derivative was chosen to best configure with the MIP and testing solvent system. NBD is known to be highly sensitive to its environment. The amines present in NBD have low to no fluorescence in water and a variable emission spectra and quantum yields in organic solvents. This would allow the emission of fluorescent light when present in ethanol and completely quenched when present in water. The actual derivative was 4-(2-Acryloyloxyethylamino)-7-nitro-2,1,3-benzoxadiazole (NBD-AE), depicted in FIG. 5H, and could also be capable of hydrogen bonding with the template molecule.

The starting material of this synthesis was 4-chloro-7-nitro-2,1,3-benzoxadizole (NBD-Cl), purchased from Sigma Aldrich, and 400 mg (2 mmol) was dissolved in 80 mL of acetonitrile. Then 280 μL of ethanolamine was added to the 80 mL mixture and was stirred at room temperature for 30 minutes. The solution was then evaporated dry under reduced pressure. Dried residue was passed through a chromatography column (1 meter in length) on silica gel with dichloromethane and methanol at a 19:1 ratio. There was a 60% yield from this column presented as orange crystals. NMR was done to confirm the structure in deuterated chloroform and compared to the literature. In addition, a melting point was taken to confirm that no derivatives were present in the sample, m.p. 153° C. The product that was synthesized from this reaction was 7-nitro-2,1,3-benzoxadiazole (NBD-NH(CH₂)₂OH) and this reaction is depicted in FIG. 5I [Onoda, M. et al., (2002) Anal. Chem. 74 (16), 4089-4096].

To complete the synthesis of NBD-AE the NBD-NH(CH2)2OH product was dissolved in 15 mL of acetonitrile, 50 mg (0.22 mol), reaction observed in FIG. 5J. Acryloyl chloride, purified using a basic alumina plug, was added to the reaction mixture and then refluxed for 4 hours with stirring. This reaction mixture was then evaporated under reduced pressure until the product was dry. The residue was passed over a chromatography column made up of silica gel and ethyl acetate and n-hexane at a 1:1 mixture to produce NBD-AE at a 76% yield of orange powder. This powder was confirmed to be NBD-AE by NMR (as reported in the literature and confirmed) and melting point measurements, 131° C. [Uchiyama, S. et al., (2003) Anal. Chem., 75 (21), 5926-5935].

### Fluorophore Monomer Response to Binding of α-Tocopherol

Fluorescence spectroscopy was used to determine if the MIP sensor was capable of giving a fluorescence response, turning off its fluorescence, to binding with the template. To confirm that the MIP sensor will decrease in fluorescence intensity with the binding of the template, simple in solution testing was done by having the MIP sensor present (2000 μL of 10 g/L) in the 50/50 ethanol/water mixture with the addition of α-tocopherol (200 μL of 33 mg/L) in the same solution. This new mixture was allowed to equilibrate for an hour at room temperature. This experiment was done before the kinetics experiments described in Example 3. FIG. 5K shows results demonstrating fluorophore responses to the various environments.

The results in FIG. 5L show that the MIP sensor does in fact have a fluorescence response to binding with the template molecule. The fluorescence intensity decreases in the presence of α-tocopherol, but not as much of a decrease shown in the predicted data (FIG. 5E). It was also noted that the MIP sensor decreases in fluorescence intensity in the presence of 4-hydroxycoumarin. The selectivity of the fluorescence decrease is not as great as the difference in their respective distribution ratios. These results support a role for this type of sensor for in-solution testing.

### Results of Inkjet Printed MIP Paper-Based Sensor

Originally, the use of a piezoelectric printer was going to be employed to print this MIP sensor ink on to paper substrates. Printing is achieved by having a piezoelectric material in an ink filled chamber behind each nozzle and a voltage is applied. The piezoelectric material changes shape and generates a pressure pulse in the fluid that forces a droplet to be expelled from the nozzle [Kolm, H. H. & Kolm, E. A., (1984) Piezoelectric printer and asymmetric piezoelectric actuator used therein; Cate, D. M. et al., (2015) Lab on a Chip, 15 (13), 2808-2818]. There are several factors that affect this process and need to be accounted for so that a printable material can be created. The Ohnesorge number (Oh) is a dimensionless constant that describes the tendency for a drop to either stay as a droplet or fall apart. The formula is shown below and compares viscous forces (η) with inertial (l) and surface tension forces (σ) [Cho, K. S. & Cha, T. W., (2014) Quantum dot ink composition for inkjet printing and electronic device using the same].

$$Oh = \frac{η}{\sqrt{l} ρ σ}$$

The Z number for printing is equal to 1/Oh, therefore, viscosity here has the biggest effect on this Z number. Viscosity is the hardest to adjust because it will affect the MIP sensors capabilities; this was the first parameter to be measured [Teichler, A. et al., (2013) European Polymer Journal, 49 (8), 2186-2195]. Table 5C provides the listed viscosities of the MIP sensor at different concentrations. A viscosity of 32 Pascal second could be optimal in the preparations described herein.

TABLE 5C
provides MIP sensor at various concentrations.

| MIP Sensor Concentration (g/L) | Viscosity in mPa · s |
|---|---|
| 0.1 | 1.17 |
| 10 | 1.33 |
| 100 | 1.56 |

When the optimization of the printing ink was done it was quickly identified that the MIP ink was not ideal for this type of printing method. The viscosity parameters of the MIP sensor, indicated that just increasing the concentration was not enough to increase the viscosity. The measured viscosity of the different MIP sensor concentrations are at mPa·s and they need to be at least an order of magnitude higher at Pa·s. Further studies provide information on incorporation of additional of surfactants and other additives for optimization.

Studies were performed to assess use of MIPs of the invention with a thermal ink jet printing technique. Thermal inkjet printing works by several tiny chambers, that each contain a heater, filled with ink. A pulse of current is passed through the heaters which causes a rapid vaporization of the ink with in the chamber. The vaporized ink forms a bubble which causing a large pressure increase and propels a droplet of ink out of the cartridge. Inks are usually water based and have some type of volatile component to form the vapor bubble. Surface tension of the ink is the only parameter that needs to be controlled to allow successful printing [de Gans, B. J. et al., (2004) Advanced Materials, 16 (3), 203-213].

Isopropyl alcohol was added to the MIP sensor ink in order to utilize the thermal inkjet printing technique [Singh, M. et al., (2010) Advanced Materials, 22 (6), 673-685]. Before the MIP sensor ink can be printed within the designed channel, the outlined sensor had to be printed and treated first. The design of the outlined microfluidic paper based sensor was completed with Microsoft PowerPoint. An A4 size filter paper was cut accordingly and feed through a ColorQube 8570 wax printer. A layer of wax ink was also applied to the back side of the printer to ensure that the MIP ink and subsequent sample would not soak through the paper. The paper was heated with slight pressure at 180° C. for two minutes with the back side face down to ensure that all the wax ink was absorbed through the paper [de Gans, B. J. et al., (2004) Advanced Materials, 16 (3), 203-213]. A thermal Canon iP2700 inkjet printer (Canon, Tokyo, Japan) was used to apply the MIP sensor ink within the waxed outlined sensor channel. The Canon printer cartridge was cut open to remove the ink and the sponge present inside. Several flushes of water were used to properly clean the cartridge of all the black ink present [Yamada, K. et al., (2015) ACS Applied Materials & Interfaces, 7 (44), 24864-24875; Li, X. et al., (2010) Colloids and surfaces B: Biointerfaces, 76 (2), 564-570]. The MIP sensor ink, present in a 70% isopropyl alcohol solution, was added to the cartridge and the cartridge was properly sealed. The MIP sensor was printed within the channel design. The same sensor paper was passed through the printer a total of 20 times with 1 minute intervals between each run. This allowed the MIP sensor to be layered within the channel to ensure even coverage of the MIP sensor within the channel. The printed sensor was stored in a humidity controlled chamber in-between testing [Yamada, K. et al., (2015) ACS Applied Materials & Interfaces, 7 (44), 24864-24875].

FIG. 5L represents the results of thermally printing the MIP ink. A distinct amount of fluorescence is present after printing 20 layers and under UV-light allows detection to be easily done with the naked each. The printed MIP sensor was tested to see if the paper based MIP ink reacted similarly to the in solution testing and resulted in a visible sensory change when the template was present. FIG. 5M illustrates results of the sensors interaction with the solvent system, a selectivity compound, and the template.

### Summary and Overview of Results of Example 4

From the paper-based experiments, it is clear that this is not a visibly selective process. The in-solution experiments show that there is a spectral difference when the template is present. The development of a MIP for α-tocopherol was fully optimized and had the highest distribution ratio of any MIP developed in these experiments. The selectivity of the MIP is also incredibly high when compared to how much the MIP binds that template. In solution testing showed that this MIP works best when it is allowed to freely move. However, if this MIP could coat a substrate by having one end anchored and still allowed to somewhat move, removal of the template is expected to be a shorter process. The binding of the MIP is expected to also occur at these high affinities because they would still be allowed to go through a thermal phase transition.

### Example 5

### Attachment of a RAFT MIP to a Gold Nanoparticle

### Reducing RAFT MIP and Attachment to Gold Nanoparticles

Studies described in previous Examples indicated that the MIP was not attached to the paper surface and therefore was lifted off the paper and, through capillary action, traveled upwards. The wax boundary was the only factor that kept the MIP ink within the channel. When the sample was dropped in the sampling area the water based sample was wicked up the channel. As it reached the MIP printing area, the fluorescent polymer traveled with the sample liquid up the channel and pooled at the top of the sensor. The MIP was not allowed to interact with the sample long enough and the MIP fluorescent light was diluted by the sample volume, exposing less light. This was seen across all samples and the blank (water).

It was determined that to improve the MIP function, it would be anchored to a substrate. Many analytical protocols, including most immunoassays, involve reagents bound to a solid substrate. Therefore, having the sample flow over it would allow enough time to bind with the template. In addition, anchoring the MIP to a substrate would also allow for a binding constant determination. If a known template solution was exposed to the anchored MIP, and then removed, the remaining solution could be analyzed, revealing the exact amount bound. This is unlike the equilibrium dialysis experiments that have an equilibration factor involved.

One approach was to attach the prepared MIPs of the invention to metal nanoparticles. Thiol end groups of a polymer have been shown to stabilize AuNPs in water [de La Fuente, J. M. et al., (2001) Angewandte Chemie (International ed. in English), 40 (12), 2257-2261]. Various other water soluble homopolymers and copolymers have been shown to stabilize AuNPs by physical adsorption [Mayer, A. B. R. & Mark, J. E., (1998) European Polymer Journal, 34 (1), 103-108].

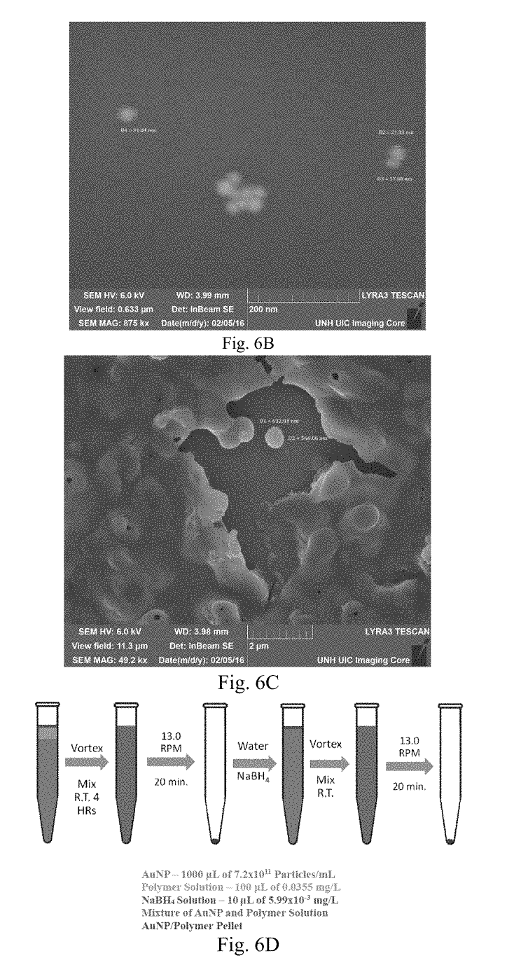

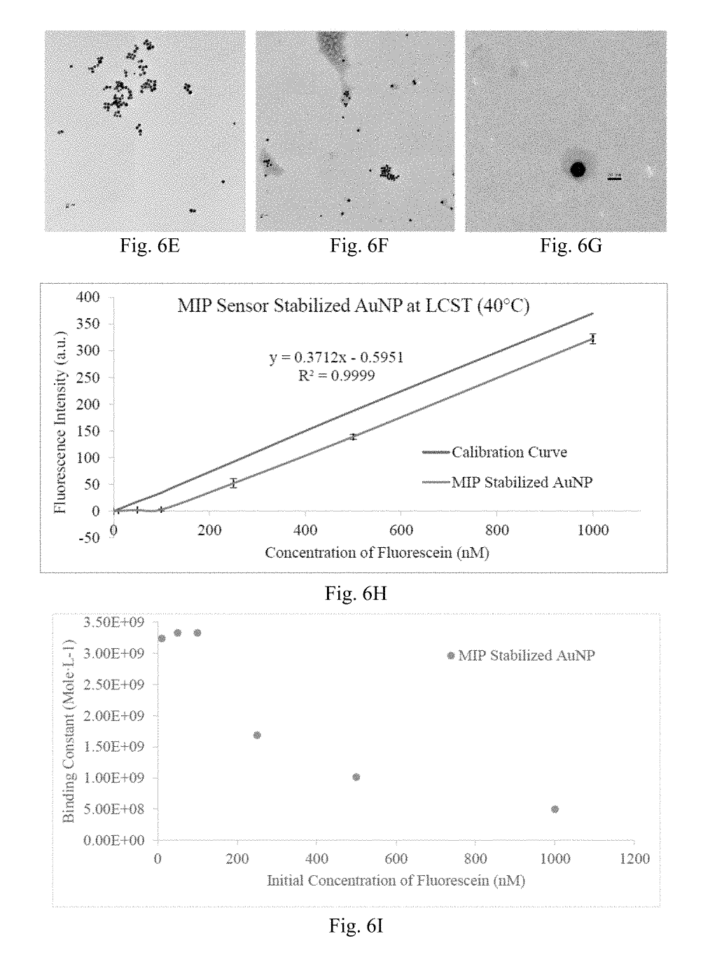

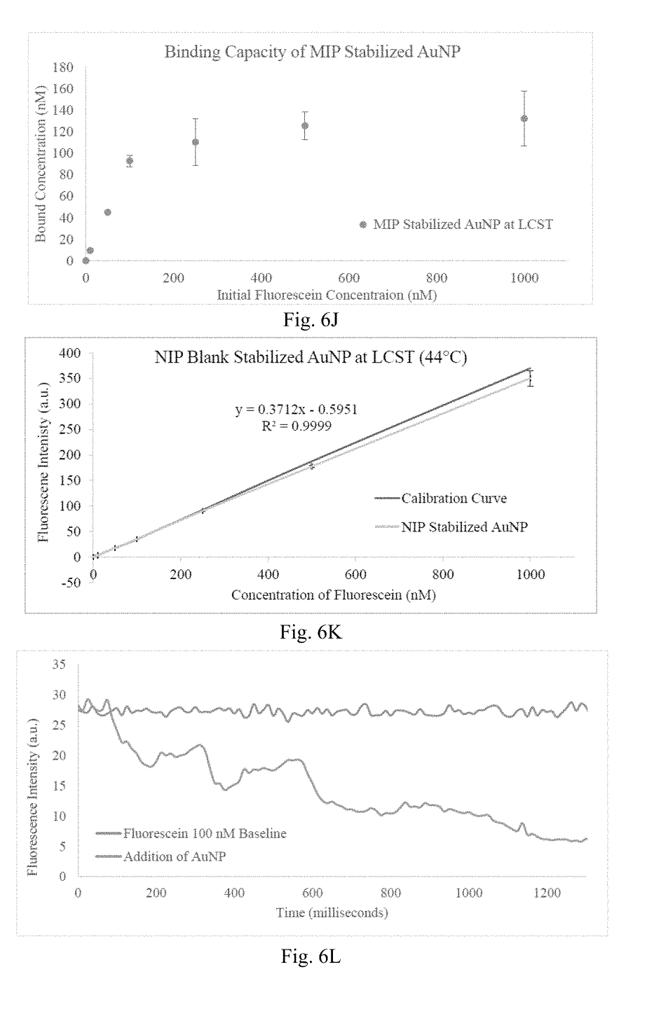

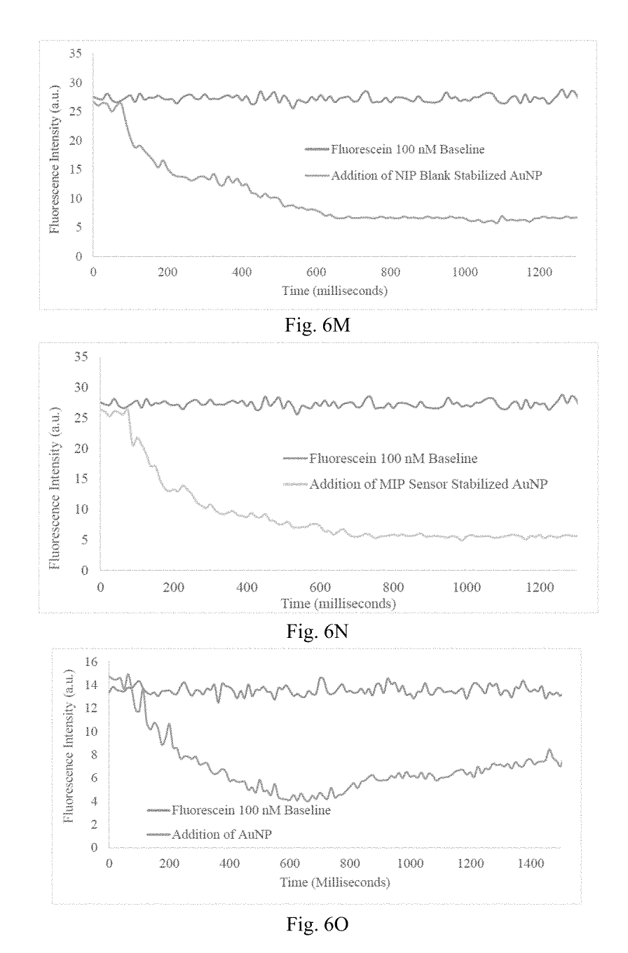

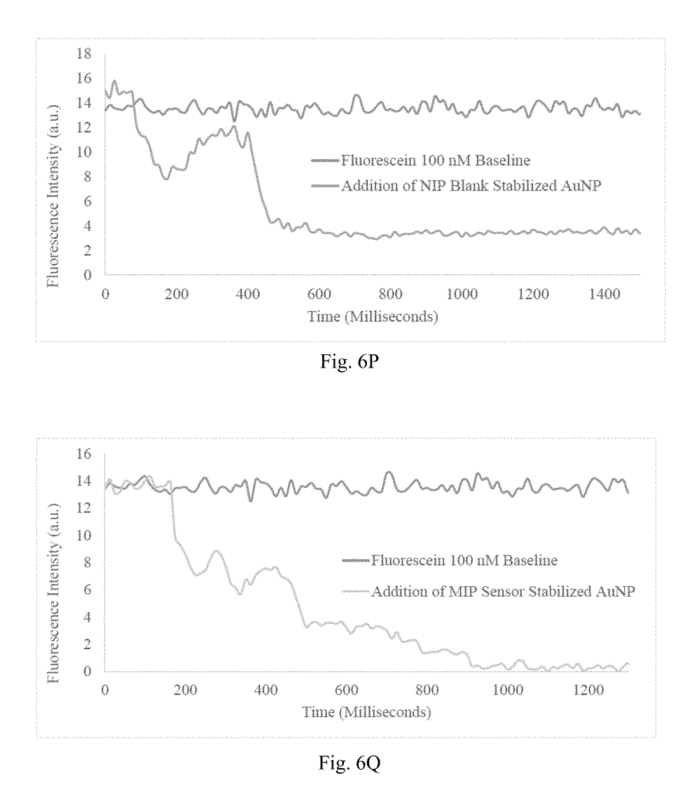

Various of the MIPs described herein were prepared by RAFT and bear dithioester end groups. Reducing these dithioester end groups to thiols in an aqueous media in the presence of a suitable metal, sol, will lead to formation of copolymer stabilized metal nanoparticles [Lowe, A. B. et al., (2002) J. Amer. Chem. Soc., 124 (39), 11562-11563]. Thiol groups bind to the gold surface with high affinity, forming a Au-sulfur bond. The chemistry is presented in FIG. 6A.

When the prepared MIPs were attached to AuNPs, they could be spun down and removed from solution, thus enabling both binding constant measurements and sensing and separation applications [Daniel, M.-C. & Astruc, D., (2004) Chem. Rev., 104 (1), 293-346].

### Manufacturing MIP-Coated AuNP

The MIP sensor and NIP blank used in Example 3 were the same MIP/NIP used in the Example 5 experiments because they were synthesized using a RAFT agent and have been shown to bind template. Gold nanoparticles that are 20 nm in diameter and stabilized in a 0.1 mM phosphate-buffered saline (PBS) solution were purchased from Sigma Aldrich. This AuNP suspension has ˜7.2×10¹¹ particles per milliliter.

Initial attachment experiments used large volumes of AuNP (˜20 mL) solution with the same volume of polymer solution at higher concentrations, 0.2 g/L, and the addition of 10 mg of sodium borohydride. This was completed in a reaction flask with stirring overnight. Originally, it was hypothesized that a volume to volume ratio was needed to calculate the amount of polymer attached to the gold nanoparticles. To confirm that the polymer was attached to the AuNP scanning electron microscopy (SEM) was utilized [García-Negrete, C. et al., (2015) Analyst, 140 (9), 3082-3089].

A gold nanoparticle sample of 7.2×10⁸ particles per mL was prepared along with a sample of 7.2×10¹¹ gold nanoparticles with a 100 mg of polymer attached per mL. SEM uses a mounted stub of metal with an adhesive double sided carbon tape to attach samples. The liquid sample is then left to dry over-night to ensure all water and solvents have evaporated off [Okuyama, K. & Lenggoro, I. W., (2003) Chemical Engineering Science, 58 (3), 537-547]. A combination of gold and palladium are sputter coated on to the sample because it needs to be electrically conductive to prevent accumulation of electrostatic charger at the surface. However, the AuNP sample did not require this step in the preparation because it is already conductive [Potter, C., (1952) Annals of Applied Biology, 39 (1), 1-28]. The images are listed FIGS. 6B and 6C.

The results shown in FIG. 6B indicated that there is a range of AuNP size, but they average out to 20 nm the labeled size. FIG. 6C shows there was a lot more polymer than nanoparticles of gold present in the sample. The sample is just a matrix of polymer with gold nanoparticles scattered throughout. In the middle of the image is a heavy coated nanoparticle. The diameter of the AuNP has increased in size by an order of magnitude. The coating of the sample with the gold and palladium mixture does not allow use to determine what is metal and what is polymer. So first, a calculated amount of polymer, gold nanoparticles, and sodium borohydride was made to ensure that the sample exists as AuNP evenly coated with polymer. It is necessary to have less polymer added to the reaction mixture so it forms a colloidal around the nanoparticle and not a matrix of polymer like in FIG. 6C. Second, transmission electron microscopy should be used instead of SEM because it can differentiate between the gold nanoparticle and the attached polymer. The gold nanoparticle will be displayed as a dark sphere and the polymer present will look like a light gray cloud. This will help to see if the polymer is attached or just forming a matrix with particles trapped within.

The nanoparticle diameter was used to determine the amount of MIP to add in solution to stabilize the AuNP [Sau, T. K. et al., (2001) Journal of Nanoparticle Research, 3 (4), 257-261]. In addition, the correct amount of sodium borohydride should be calculated to ensure that just enough is added to reduce the dithioester RAFT end group to a thiol and not aggregate the AuNP [Brust, M. et al., (1995) J. Chem. Soc., Chemical Communications, (16), 1655-1656]. The amount of copolymer to add to the AuNP suspension was calculated as follows. The surface area of the AuNP, A=4πr², was calculated first and determined to be 2124 nm². A sulfur atom has a radius of 0.1 nm so the AuNP could theoretically fit 21237 atoms of sulfur about the surface. This was a theoretical value, however, and does not take into consideration steric effects. Subsequently, there was an estimate made that a fourth of the available addition sites could add a sulfur atom. Another estimate was applied to the calculation to consider that these thiol end group polymers would block some of the surface of the AuNP when in solution. A factor of 100 of the available sites was applied. By this calculation, each gold nanoparticle would have space for 53 polymer chains with thiol end-groups [Battocchio, C. et al., (2014) J. Phys. Chem. C, 118 (15), 8159-8168].

Discussed below are the calculated values of each chemical substituent used to manufacture MIP stabilized AuNPs. Knowing that potentially only 53 RAFT MIPs could be attached to the surface of the AuNP, and knowing the number of AuNPs, permitted calculation of the concentration of MIP. A factor of 25 sodium borohydride moles to 1 mole of RAFT agent present was applied to determine how much to use to reduce the RAFT end group to a thiol. The following amount of MIP being used was determined by the estimation of the number of binding sites per mole. All of these values are listed in Table 6A. In later experiments, the binding capacity and binding constant were measured and calculated.

TABLE 6A
Calculated values of each chemical substituent to manufacture the
MIP stabilized AuNP.

| Particles | | | | |
|---|---|---|---|---|
| Per of AuNP | | NaBH4 | Binding Sites Per | μMole |
| Solution mL | MIP Per mL | Per mL | mL | Fluorescein |
| 7.2 × 10¹¹ | 0.00355 mg | 5.99 × | 3.331 × 10⁻⁰⁵ mg | 1.00 × 10⁻⁰⁴ |
| | | 10⁻⁰⁵ mg | | |

MIPs were attached to AuNPs using a microcentrifuge tube, a vortex, and centrifuge, as shown in FIG. 6D. Aqueous solutions of AuNPs, MIP, and sodium borohydride were all added to the tube and thoroughly mixed. This solution was left at room temperature on an agitator for four hours. The microcentrifuge tube was then spun at 13000 rpms for 20 minutes. The supernatant was then removed, leaving a pellet. Then 1 mL water and the same volume of sodium borohydride was added into the tube, mixed, and then spun down again. This was done three separate times followed by the same procedure minus the sodium borohydride to rinse the AuNP of any unattached MIPs. The AuNP was spun down one final time to remove all excess water and leave just the pellet.

Visually Verifying the Stabilization of AuNP with MIPS

Transmission electron microscopy (TEM) was employed to examine the gold nanoparticles after stabilization. [Shan, C. et al., (2010) Biosensors and Bioelectronics, 25 (5), 1070-1074]. The MIP-stabilized AuNPs were prepared as described above, and then suspended in a pH 7.2 phosphate buffer and allowed to equilibrate so that the AuNPs were evenly distributed throughout the solution. An aliquot was taken and used to image the attachment of MIP to the AuNP. The aliquot was dropped on to a Formvar TEM copper grid support film. These copper grids have a thermal resin of polyvinyl formals that form the support material in between each grid. After dropping the liquid sample onto this grid, it was allowed to settle for about two minutes and then the excess liquid was wicked away. The disc was placed in a petri dish and allowed to dry overnight. The copper grid was then placed into the TEM sample holder and brought down to vacuum.

The images shown in FIGS. 6E, 6F, and 6G were created using the TEM at 10,000 and 20,000 magnifications. The AuNPs are the dark black spots. Multiple clusters of AuNPs are seen with light to dark gray material surrounding the darker spots, see FIGS. 6F and 6G. This is the presence of the MIP attached to the AuNP. Several wash and spin cycles happen during the manufacturing of these MIP stabilized AuNPs [Lee, C.-U. et al., (2010) Polymer, 51 (6), 1244-1251]. Therefore, the AuNPs left at the end of this process should only have attached MIP. The coverage of MIP about the AuNP is not a uniform process and the amount of MIP loaded varies by AuNP. However, all the AuNPs had some attached MIPs.

### Binding Experiments

The MIP-stabilized AuNPs were easily spun down into a pellet, leaving a supernatant that was comprised of just the solvent. This makes it convenient to measure the binding affinity and capacity of the MIP.

To calculate the binding constant of the MIP, various concentrations of template solution were added to each individual pellet tube. Each tube was then sonicated to ensure AuNPs are not aggregated together in solution and allowed to interact with the MIP stabilized AuNPs. The MIP and NIP solutions were then heated to the LCST, 40° C. and 44° C. respectively, and held there for 30 minutes to equilibrate the temperature.

Calculating the amount of fluorescein present in the supernatant was done similarly to the equilibrium dialysis experiments. The supernatant was removed and analyzed by fluorescence spectroscopy. The fluorescence intensity was then used to calculate the concentration of fluorescein using the calibration curve. The results are seen in FIG. 6H. The difference of the fluorescence intensity from the initial concentration of the fluorescein solutions to the MIP stabilized AuNP supernatant is the amount of fluorescein bound to the MIP.

A binding constant is a special case of the equilibrium constant, K_{Binding}. It deals with the binding and unbinding reaction of the polymer binding site (receptor) and the template molecules (template), which is formalized as: Poly+T⇄PolyT. Here in the reaction [Poly], [T], and [PolyT] represent the concentration of the unbound free binding sites, the concentration of unbound template molecule, and the concentration of the MIP and template complex once bound, respectively [Zhang, Y. et al., (2007) The Journal of Physical Chemistry C, 111 (25), 8916-8924].

Calculation of Binding Constant: the following is an example calculation of MIP attached AuNP with 10 nM fluorescein spin down experiment:

$$10 nM Fluorescence Intensity Before ( a . u . ) - Fluorescence Inentsity After ( a . u . )$$

$$3.249 - 0.378 = 2.871$$

$$\frac{Difference in Fluorescence Intensity}{Slope of the Calibration Curve} + Y intercept = M I P Bound Fluorescein = \frac{2.871}{0.3712} + 0.5951 = 8.329 nM = 8.329 \times {10}^{- 9 } M$$

$$Fluorescein Present = 1.00 \times {10}^{- 8} M$$

$$\begin{matrix}M I P Present = 1.00 \times {10}^{- 4} L \times 3.55 \times {10}^{- 5} \frac{grams}{L} \\ = \frac{3.55 \times {10}^{- 9} grams}{13 , 800 \frac{grams}{mole}} \\ = \frac{2.57 \times {10}^{- 13} moles}{0.001 L} \\ = 2.57 \times {10}^{- 18} M\end{matrix}$$

$${K}_{Binding} = \frac{M I P Bound Fluorescein}{Fluorecein \times M I P}$$

$${K}_{Binding} = \frac{8.329 \times {10}^{- 9} M}{1.00 \times {10}^{- 8} M \times 2.57 \times {10}^{- 10} M} = 3.09 \times {10}^{9}$$

Using this equation, the binding constant could be calculated for each centrifuge experiment. From the GPC data a molecular weight is not available and moles of polymer present in the MIP-stabilized AuNP solution can be calculated. Initial fluorescein concentration is known. Using FIG. 6H, the amount of bound fluorescein is known. These experiments have shown that it is possible to obtain a binding constant on the order of 10⁹ Mole·L⁻¹. The amount of fluorescein being bound or free in solution is very small (10 nM). FIG. 6I shows that the binding constant eventually decreases with the addition of more fluorescein, depicting that the MIP has reached its binding capacity. These results are expected if there is a distribution of binding sites with different affinities. This is also seen in nature; normal antibodies have a distribution of binding constants. Monoclonal antibodies are prepared by making multiple copies of a particular antibody to get a uniform antibody with a single binding site [Riechmann, L. et al., (1988) Nature, 332 (6162), 323-327]. These binding constants are higher than other MIP reported binding constants [Shumyantseva, V. V. et al., (2015) Doklady Biochemistry and Biophysics, 464 (1), 275-278; Ansell, R. J., (2015) Molecularly Imprinted Polymers in Biotechnology; Springer International Publishing; pp 51-93; EL-Sharif, H. F. (2014) Physical Chemistry Chemical Physics, 16 (29), 15483-15489]. These binding constants are comparable to binding constants of antibodies that are capable of picomolar selectivity [Im, H. et al., (2014) Nature Biotechnology, 32 (5), 490-495].

MIP-stabilized AuNP binding capacity, (see FIG. 6J), was compared to the theoretical binding capacity. Results in Example 3 described the calculations of the theoretical binding sites. This MIP-stabilized AuNP solution should be able to theoretically bind 100 nM of fluorescein per mL. The binding capacity is the maximum amount bound which would be amount where the levels off at high fluorescein. A comparison of the theoretical to the actual amount, 132 nM, suggested two possible scenarios: that the MIP is entangling some fluorescein randomly with the phase transition of the polymer, or there is more 4-VP being polymerized within the MIP that is creating more binding sites (see Example 3, NMR results). With the feed ratio just being an approximation and not the actual percentage, it is possible that more binding sites are being created [Canning, S. L. et al., (2016) Macromolecules, 49 (6), 1985-2001].

The NIP blank was attached to an AuNP as well for the same experiment. In FIG. 6K, are the results of that experiment and no binding occurred. The original fluorescein signal given by the fluorescein solutions introduced to the NIP blank AuNP was the same observed signal after the spin-down experiment. This was indicative of fluorescein not being bound by the non-imprinted polymer. In addition, the statement that the fluorescein signal is not affected by any gold interactions or being entrapped by the polymer is also true. This clarifies that all signal loss is actually fluorescein being bound to the MIP and proves that the concentration bound by to be accurate.

### Stabilized AuNP Binding Kinetics

The binding experiments described above showed that the MIP stabilized AuNP is capable of binding on a 10¹² order of magnitude. Using a AuNP to anchor the MIP has shown that it is still capable of a high binding affinity; however, a concern is that the binding kinetics will be slower than they are in solution. If the AuNPs aggregate, they could block MIP binding sites on surrounding AuNPs. MIP binding sites that are within this supposed aggregate of MIP stabilized AuNPs would only allow the surface and outer AuNP MIP binding sites to bind to template. The same kinetics experiments that were done in chapter five were repeated using the MIP stabilized AuNP solution, 7.2×10 particles with 0.0035 mg of MIP per mL. These solutions have less MIP present than what has been previously tested.

The kinetics of the MIP stabilized AuNP reaction with template is similar to the MIP in solution; however, it was noticed that just the gold nanoparticles quench the fluorescence intensity at room temperature and the LCSTs, FIGS. 6L and 6O. The fluorescence intensity decreases by more than 50% of the baseline line taken before the addition of the AuNPs. Doubling the volume by the addition of the AuNP solution will decrease the fluorescence signal by half. Any further signal decreased has been contributed to binding once the gold quenching was subtracted from the total intensity lose. Fluorescein was not templated to the gold nanoparticle, but the AuNP absorbs light around 512-524 nm. It is absorbing the emission of light given off by the template [Sperling, R. A. et al., (2008) Chemical Society Reviews, 37 (9), 1896-1908]. This was taken into consideration when observing the rest of the kinetic results. AuNPs were observed to decrease signal by 2 or 3 arbitrary fluorescence units. Specifically, the case when analyzing the NIP blank stabilized AuNP binding kinetics. The loss of fluorescence signal for this experiment is more than half of the fluorescein baseline (100 nM) at both temperatures, see FIGS. 6M and 6P. This is most likely due to the presence of the gold nanoparticles. Less fluorescence signal was observed when compared to results from chapter four of the MIP sensor stabilized AuNP, see FIGS. 6N and 6Q.

The binding constant reaction is defined by the on-rate constant, Kₒₙ, and the off-rate constant, K_{off}, which have units of inverse moles per seconds and inverse seconds. The forward binding transition Poly+T→PolyT are balanced by the backward unbinding transition PolyT→T+Poly. The reaction is represented by Kₒₙ [Poly] [T]=K_{off} [PolyT] [Pan, A. C. et al., (2013) Drug Discovery Today, 18 (13), 667-673]. The binding constant is defined by:

$$constant {K}_{Binding} where {K}_{Binding} = \frac{{K}_{on}}{{K}_{off}} = \frac{[ PolyT ]}{[ Poly ] [ T ]}$$

[see Hulme, E. C. & Trevethick, M. A., (2010) Brit. J. Pharmacol., 161 (6), 1219-1237]. The kinetics experiments show that the on rate of the template binding is very fast and the off rate can be assumed from the binding constants to be fast as well. This was not the same off rate as seen in the removal process. The removal process, discussed above, was long due to the entanglement of MIP polymers and entrapment of template. The amount of MIP present on the AuNP was an order of magnitude less, but still capable of binding a noticeable amount of template while being stabilized on AuNPs.

### Summary and Overview of Results of Example 5

The RAFT polymerized MIP can be attached to a AuNP by a simple reduction in water. Less MIP had to be used than before because of the limitation of AuNP present in the solution. This was thought to lower recognition of fluorescein. This was found to not be the case with the prepared MIP, which had a binding constant that was two orders of magnitude higher than the previous in solution testing with equilibrium dialysis. Using less MIP attached to gold nanoparticles supported a conclusion that less concentrated solutions allowed the polymer to be in a conformation that exposed more binding sites. The obtained binding constants were comparable to those of biomolecules and increased by a couple orders of magnitude, when compared to Example 3 experimental results. The binding capacity of fluorescein was reduced to half of what the MIP could bind in solution, (results Example 3). However, when considering that the MIP concentration was also reduced by an order of magnitude, this also supports a conclusion that less concentrated solutions allowed the polymer to be in a conformation that exposed more binding sites. The binding kinetics were not changed and still presented the same time scale as results from Example 3.

### Example 6

The U.S. EPA determines that phenols present in drinking water should be at <1 μg/L.35. Previous methods for removing these phenolic compounds include adsorption on activated carbon, chemical oxidation, microbial degradation, and electrochemical methods. These techniques are not only expensive, but toxic in themselves. Using a poly (NIPAm) based MIP can be a viable and low cost method to remove these phenolic compounds from a water medium. 4-Nitrophenol was used as a template molecule and 2 and 3 nitrophenol was used for selectivity experiments (FIG. 1H). The recognition monomer used in this system was 4-vinylpyradine (4VP) because it better matches the hydrogen bonding conditions of the phenolic compound. RAFT polymerization was also used to control chain length, resulting in more uniform chain lengths. Traditional free radical polymerization was used for a proof of concept for the various optimization changes in the polymer network.

This is accomplished by using NIPAm as the main backbone monomer along with acid (methacrylic acid) and base (4-vinylpridine) monomers to form non-covalent crosslinks (interactions). The addition of these types of crosslinks has been shown to help keep the size and shape of the templated site, but still remain as an aqueous solution forming a hydrogel.

The prepared MIP hydrogel need to have an exceedingly larger volume change, collapsing, with the amount of template concentration bound. The acid and base monomers will interact about the polymer chain to form non-covalent hydrogen bonds, aiding in keeping the binding sites the same size and shape of the templated molecule. The addition of low levels of a covalent crosslinker, N,N′-methylenebisacrylamide (MBAm), was applied to help keep the same formed acid and base monomer pairs within reach of one another after polymerization. During the removal process of the template molecule, it is necessary to change the pH and possibly the temperature of the removal solution. Some covalent crosslinks throughout the polymer will allow for the removal of the template. Once placed into a deionized water system, the non-covalent crosslinks can go back to interacting and reform the binding site that was once templated. An excess (4-10 mol %) of MAA or 4-VP should be used as the recognition monomer that forms the templated cavity, so it does not have to compete with the multiple interactions.

The studies described herein were also used to determine type and amount of crosslinking and the rest of the polymerization system for optimal recognition. The polymer composition was synthesized in varying ratios of different copolymers stated later, for example in Examples 1-3. Once an optimal polymer network was discovered, it was found that the nitrophenol template was not suitable for absorbance measurements at the low concentrations that the MIP could sense. A new template molecule needed to be chosen so that binding experiments, kinetics, and selectivity could be determined at nanomolar concentrations (FIG. 1I).

Fluorescein is a fluorescent molecule that has an excitation maximum at 594 nm and an emission maximum at 514 nm. This compound has measureable fluorescence at a 10 nanomolar concentration and can be successfully imprinted using 4VP as a recognition monomer. This molecule was used to show how fast binding occurs in an aqueous environment and how sensitive the MIP is at low concentrations. Fluorescein was used to test the MIP capabilities to see how the MIP reacted to different environments. When system optimizations were made, this template molecule may be switched out for a template molecule that is needed for a sensing application.

### Applications of Molecualry Imprinted Polymer Sensors

MIPs of the invention have been prepared based on poly(N-isopropylacrylamide) poly (NIPAm). The MIP preparation methods described herein offer an alternative approach to conventional MIP preparation. Poly (NIPAm) undergoes a thermal phase transition. It is soluble in water at low temperatures due to hydrogen bonding between the amide functional group and water, but comes out of solution at higher temperatures. The transition temperature is known as the lower critical solution temperature (LCST). Above this temperature, hydrophobic interactions between isopropyl groups cause the copolymer to assume a globular formation. Lightly crosslinked imprinted poly (NIPAm) copolymers have been shown to bind template at and above the LCST (see for example: [Watanabe, M. et al., (1998) J. Am. Chem. Soc. 120 (22), 5577-5578; Alvarez-Lorenzo, C. et al., (2000) Macromolecules 33 (23), 8693-8697; Yu, C. & Mosbach, K., (2000) J. Chromatog. A 888 (1-2), 63-72]). The hydrophobic interactions between isopropyl groups apparently serve as noncovalent crosslinks that result in a selective binding site. The low percentage of covalent crosslinks may serve to guide poly (NIPAm) collapse so that these sites reform above the LCST even after the temperature has been lowered below the LCST, causing the copolymer to assume a random coil configuration in aqueous solution.

An optimal polymer network for sensing a specific template molecule was determined. The network was used throughout a number of experiments described in the Examples section, with varying amounts of non-covalent crosslinking in order to determine if this will affect binding and selectivity while still presenting an aqueous polymer. Additional studies were performed using application-based template molecules such as vitamin E (alpha tocopherol) and vitamin C (ascorbic acid).

In solution, experimental results revealed that a prepared MIP of the invention was suitable use as a chemical sensor and was combined with the application of ink jet printing (FIG. 1J) and anchoring MIPs to gold nanoparticles (FIG. 1K). The prepared MIP compound was further investigated and it was confirmed that MIP compounds of the invention, could be used to prepare a viable sensor array. Experiments were also carried out that confirmed the binding properties of the polymer and to develop an appropriate sensor array for a point of care and in situ chemical testing. Experiments were also performed that demonstrated attachment of a prepared MIP of the invention to a gold nanoparticle in the presence of water using sodium borohydride (FIG. 1K, top). Attachment of MIP spin coated on to a gold coated glass slide with sodium borohydride (FIG. 1K, bottom)

Studies were also performed to combine the use of an aqueous molecular imprinted polymer (MIP) solution templated for ascorbic acid and the functionalization of a gold coated glass slide (FIG. 1K). The MIP network is synthesized using a living polymerization technique and a new non-covalent crosslinking system, acid and base crosslinking, allowing for an aqueous polymer solution that has functionalized end groups.

### Overall Results from Examples

The molecular imprinted polymer formulations described in the preceding Examples, were optimized for better imprinting of the template and higher template recognition. This has been demonstrated by the increasing binding affinity that was attained, as presented in the Examples. The MIP does not only have a high affinity and selectivity for the template, but it also does this rapidly. Binding with the template molecule within seconds is a valuable property for both separations and sensing applications.

### Equivalents

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated in their entirety herein by reference.

What is claimed is:

1. A molecularly imprinted polymer (MIP) compound, wherein the MIP compound comprises a backbone monomer, two or more independently selected functional monomers, and wherein the MIP compound comprises one or more independently selected non-covalent crosslinks and one or more covalent crosslinks.

2. The MIP compound of claim 1, wherein the non-covalent crosslinks are independently selected from: an acid-base crosslink and a hydrophobic crosslink.

3. The MIP compound of claim 1, wherein the percentage of the total crosslinks in the MIP compound that are covalent crosslinks is between 1% and 7%.

4. (canceled)

5. The MIP compound of claim 1, wherein the backbone monomer is N-isopropylacrylamide (NIPAm).

6. The MIP compound of claim 1, wherein functional monomer is 4-vinylpyridine (4-VP) or methacrylic acid (MAA).

7-9. (canceled)

10. The MIP compound of claim 1, wherein the MIP compound is an aqueous compound.

11. The MIP compound of claim 1, wherein the molecular imprinting of the MIP is against a template molecule, wherein the template molecule comprises a target compound or functional fragment thereof and the MIP compound selectively binds the target compound.

12. The MIP compound of claim 1, wherein the MIP compound further comprises a functionalized end group.

13. The MIP compound of claim 12, wherein the functionalized group comprises a dithiolester.

14. (canceled)

15. The MIP compound of claim 1, wherein the MIP compound is attached to a substrate.

16-18. (canceled)

19. The MIP compound of claim 1, further comprising a detectable label.

20. (canceled)

21. The MIP compound of claim 11, wherein the target compound comprises an organic molecule.

22-24. (canceled)

25. The MIP compound of claim 1, wherein a lower critical solution temperature (LCST) of the MIP compound is between: 28° C. and 38° C.

26-28. (canceled)

29. The MIP compound of claim 11, wherein the binding of the MIP compound with the target compound alters one or more physical characteristics of the MIP compound.

30-31. (canceled)

32. The MIP compound of claim 1, wherein the MIP compound is in a solution comprising a plurality of the MIP compounds.

33-34. (canceled)

35. A method of preparing a molecularly imprinted polymer (MIP) compound of claim 1 comprising:
(a) preparing a pre-polymerization solution comprising: a backbone monomer, two or more independently selected functional monomers, and a solvent;
(b) adding a template compound to the prepared pre-polymerization solution;
(c) polymerizing the template/pre-polymerization solution to form a MIP compound;
(d) separating the MIP compound from the template compound; and optionally
(e) lyophilizing the separated MIP compound.

36. The method of claim 35, wherein the backbone monomer is a N-isopropylacrylamide (NIPAm) monomer.

37-46. (canceled)

47. The method of claim 35, wherein a means of polymerizing comprises a reversible addition-fragmentation chain-transfer (RAFT) method.

48. (canceled)

49. The method of claim 35, wherein the percentage of the total crosslinks in the MIP compound that are covalent crosslinks is between 1% and 7%.

50-52. (canceled)

53. A method of identifying the presence or absence of a target compound in a sample, comprising:
(a) contacting a sample with a MIP compound of claim 1, wherein the MIP compound selectively binds a target compound; and
(b) detecting the presence or absence of binding of the MIP compound and the target compound in the sample, wherein the presence of binding of the MIP compound in the sample identifies the presence of the target compound in the sample and the absence of binding of the MIP compound in the sample identifies the absence of the target compound in the sample.

54-55. (canceled)
